SEQUENCE LISTING (6 sequences)

SEQ ID NO: 1 - protein, 20 aa, Homo sapiens
MVRGEATARTEEAMETVFTT

SEQ ID NO: 2 - DNA, 60 bp, Homo sapiens
atggtgagaggagaggccaccgcacgaacggaagaagcgatggagacggtctttacgacc

SEQ ID NO: 3 - protein, 15 aa, Homo sapiens
MVRGEATARTEEAMC

SEQ ID NO: 4 - DNA, 8916 bp, Homo sapiens
  Source (1)..(8916): Source of sequence NHIP-peptide-eGFP
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctcagcggaacacacggtcccaaaaggcccttcccaagatggccggggcttccgcctgccgccctccctcccgtggtgctgagcgcaggctgggcgcggccacatcaccctaagggcgtgggtgtcggagtcttgcaggctggggtcccaggtgagagatgccagcagggagctctagatggggaactgggtgagcctggctcggtaagggcgtgctcagaacaccccatccccgaacacggtcttgtgtgataaaatgtcacgcaagaagaatctgaaaccgcgagaagaggaggacacggccagactgcaccgaaccccggcacctctgtggggaaaagcaggtcaggctgagcggggccggcggggacacgcgctcctgggcttctccagagtctgccggggccgggcccgggaccgaggctgggacgcgctgtgcagtcccacccctcacacccctcgcacgcctggaaacaccctcggggtaacacaagccggggttgagtttcttgaagagaagctggctgcatcctggagccagggaaagaggagcacaggggccaagcggtccagggccacggaggagcaggacccctggggaaaggcccgggtctaggccggggccaggggaccgtggtggagacctcaaaaatggcagaacacggaagcagggcgagaaaagtaaacgtagtccttgcggcagtttgaaatacacgcgggtaaacgctgggtgactccgcccggatgcagagtgggggtctgtgtctctccccacaggctgcagggaccgggctctgggtaaccagcagaaggtaacagaacgaggctgcttttcctccaggctgttctggtgtccgcgcgtggcttgtgcgctgactcctgacttggagcgccgcgtggccagagaaatctgggtgcctccaggccaccatggtgagaggagaggccaccgcacgaacggaagaagcgatggagacggtctttacgacctctggtggcggaggctcgggcggaggtgggtcgggtggcggcggatcagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt

SEQ ID NO: 5 - DNA, 7033 bp, Homo sapiens
  Source (1)..(7033): Source of sequence  NEG-eGFP
gatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaa

SEQ ID NO: 6 - DNA, 1674 bp, Homo sapiens
  Source (1)..(1674): Source of sequence Genbank QPKN01007947.146099-47772
taagaaaactcctgctctccctcctctccctctccctcctctccctctccctctccctctccctctccccacggtctccctctccccacggtctccctctccctctctttccacggtctccccctgatgctgagccgaagctggactgtactgctgccatctcggctcactgcaacctccctgcctgattctcctgcctcagcctgccgagtgcctgcgattgcaggcgcgtgccgccacgcctgactggttttcgtatttttttggtggagacggggtttcgctgtgttggccgggctggtctccagctcctaatcacgagtgatccgccagccttggcctcccgaggtgccgggattgcagacggagtctcgttcactcagtgctcaatggtgcccaggctggagtgcagtggcgtgatctcggcttgctacaacctccacctcccagccgcctgccttggcctcccaaagtgccgagattgcagcctctgcccggccgccaccccgtctgggaagtgaggagcgtctctgcctggccgcccatcgtctgggacgtgaggagcccctctgcctggctgcccagtctggaaagtgaggagcgtctctgcccggccgccatcccatctaggaagtgaggagcgcctctgccaggccgcccatcgtctgagatgtggggagcgcctctgccctgccaccccgtctgggatgtgaggagcgtctctgcccggccgccccgtctgagaagtgaggagaccctctgcctggcaaccgccccgtctgagaagtgaggagcccctccgcccggcagccacaccctctgagaagtgaggagcgtctctgcctggcagccaccccgtctgggagggaggtgggggtcagccccctgccccgccagctgcccatccgggagggaggtggggggtcagccccccgcccggccagccgcctcgtccaggaggtgaggggcgcctctgcccggccgcccctactgggaagtgaggagcccctctgcctggccagccgccccgtcggggagggaggtggggggacagccccccgcccggccagccgccccgtccgggaggtgaggggcgcctctgcccggccgcccctactgggaagtgaggagcccctctgcccggccaccaccccgtctgggaggtgtactcaacagctcattgagaacgggccatgatgacaatggcggttttgtggaatagaaaggggggaaaggtggggaaaagattgagaaatcggatggttgccgtgtctgtgtagaaagaggtagacatgggagacttttcattttgttctgtactaagaaaaattcttctgccttgggatcctgttgatctgtgaccttacccccaaccctgtgctctctgaaacatgtgctgtgtccactcagggttgaatggattaagggtggtgcaagatgtgctttgttaaacagatgcttgaaggcagcatgctcgttaagagtcatcaccactccctaatctcaagtacccagggacacaaacactgcggaaggccgcagggtcctctgcctaggaaaaccagagacctttgttcacttatctgctgaccttccctccactattgtcctgtgaccctgccaaatccccctctgcgagaaacacccaagaatgatcaataaaaaaaaaaaaaa

(19) United States
(12) Patent Application Publication
(10) Pub. No.: US 2025/0137050 A1
(43) Pub. Date: May 1, 2025

(54) METHODS AND COMPOSITIONS FOR DETERMINING RISK OF AUTISM SPECTRUM DISORDERS

(71) Applicant: The Regents of the University of California, Oakland, CA (US)

(72) Inventor: Janine M. LaSalle, Davis, CA (US)

(73) Assignee: The Regents of the University of California, Oakland, CA (US)

(21) Appl. No.: 18/683,978

(22) PCT Filed: Jun. 17, 2022
(86) PCT No.: PCT/US2022/034054

§ 371 Date: Feb. 15, 2024

Related U.S. Application Data
(60) Provisional application No. 63/234,545, filed on Aug. 18, 2021.

(51) Int. Cl. C12Q 1/6883; A61K 31/7105; A61K 38/17; C12N 15/11; C12Q 1/6844; C12Q 1/6869

(52) U.S. Cl. CPC C12Q 1/6883; A61K 31/7105; A61K 38/1709; C12N 15/11; C12Q 1/6844; C12Q 1/6869; C12Q 2600/154

(57) ABSTRACT

Described are methods for identifying an ASD risk gene, NHIP, and methods for determining the risk of an offspring for developing an ASD. A common structural variant disrupting the proximity of NHIP to a fetal brain enhancer was associated with NHIP expression and methylation levels and ASD risk, demonstrating a common genetic influence. NHIP is a novel environmentally-responsive ASD risk gene relevant to brain development in a previously under characterized region of the human genome.

### CROSS-REFERENCE TO RELATED APPLICATION

This application claims priority to U.S. Provisional Application No. 63/234,545, filed Aug. 18, 2021, the disclosure of which is hereby incorporated by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Grant Nos. AR110194, ES011269, ES021707, and ES025574, awarded by the National Institutes of Health (NIH). The Government has certain rights in the invention.

### BACKGROUND

Autism spectrum disorders (ASD) are a set of neurodevelopmental disorders diagnosed in early childhood and are classified by a loss of abilities in social interaction, social communication, and the presence of repetitive and restricted interests and behaviors. Currently, ASD affects about 1 in 68 children in the United States (US), with an estimated cost to society at a staggering $240 billion per year. Current therapeutic interventions available for ASD are behaviorally directed or symptom-based pharmacological treatments applied only after diagnosis. Little is known about the cause of ASD and while certain therapeutic approaches applied following early diagnosis have shown promise, no preventive alternatives currently exist.

ASD involves complex genetics interacting with perinatal environment, complicating the identification of common genetic risk. The epigenetic layer of DNA methylation shows dynamic developmental changes and molecular memory of in utero experiences, particularly in placenta, a fetal tissue discarded at birth. However, current array-based methods to identify novel ASD risk genes lack coverage of the most structurally and epigenetically variable regions of the human genome.

### BRIEF SUMMARY

In one aspect, the disclosure provides a method for determining a risk of an offspring for developing an autism spectrum disorder (ASD). In some embodiments, the method comprises detecting, in a biological sample obtained from the offspring, mother or potential mother of the offspring, expression and/or DNA methylation of a neuronal hypoxia inducible, placental associated (NHIP) gene, wherein decreased expression and/or decreased methylation of the NHIP gene compared to a control sample indicates an increased risk of the offspring for developing an ASD.

In some embodiments, the method further comprises obtaining a biological sample from the mother or potential mother.

In some embodiments, the biological sample is selected from the group consisting of blood, serum, plasma, or saliva from the mother, and placenta, cord blood, blood, saliva and brain from the offspring.

In some embodiments, the mother or potential mother has a child with an ASD.

In some embodiments, the mother or potential mother has a familial history of ASD.

In some embodiments, the offspring is a fetus or child.

In some embodiments, the control sample is selected from a mother or potential mother having offspring without an ASD or offspring exhibiting typical development.

In some embodiments, the detecting step comprises detecting DNA methylation of the NHIP genetic locus, the chr22q13.33 hypomethylated block, or both.

In some embodiments, lower or decreased DNA methylation levels indicates an increased risk of the offspring for developing an ASD.

In some embodiments, detecting expression of the NHIP gene comprises detecting an RNA expressed by the NHIP gene or a peptide encoded by the RNA.

In some embodiments, the RNA is transcribed from an open reading frame comprising the DNA sequence

(SEQ ID NO: 2)
ATGGTGAGAGGAGAGGCCACCGCACGAACGGAAGAAGCGATGGAGACGG
TCTTTACGACC.

In some embodiments, detecting an RNA expressed by the NHIP gene is selected from amplifying the RNA, quantifying the RNA, or sequencing the RNA.

In some embodiments, detecting a peptide encoded by the RNA is selected from i) contacting the peptide with a primary antibody that binds the peptide and detecting the primary antibody with a labeled secondary antibody, ii) linking the peptide to a detectable label, or iii) by immunostaining.

In some embodiments, the peptide comprises the amino acid sequence

(SEQ ID NO: 1)
MVRGEATARTEEAMETVFTT.

In some embodiments, the method further comprises administering a vitamin to the mother or potential mother if the mother is homozygous for a structural variant inserted about 15 Kbp upstream from the start site of the chr22q13.33 hypomethylated block. In some embodiments, the vitamin is administered during the first month of pregnancy. In some embodiments, the vitamin comprises a (e.g., one or more, or a plurality of) dietary methyl group(s).

In some embodiments, the NHIP gene is hypomethylated.

In some embodiments, the biological sample is homozygous for a structural variant insertion (chr22: 49029657, hg38) upstream of the 22q13.33 locus.

In another aspect, the disclosure provides a method for detecting an NHIP peptide in a subject. In some embodiments, the method comprises:
obtaining a biological sample from the subject; and
detecting the presence of the NHIP peptide by contacting the biological sample with an anti-NHIP antibody and detecting binding between the NHIP peptide and the antibody.

In some embodiments, the subject is a mother or potential mother of an offspring at risk for developing an ASD.

In another aspect, the disclosure provides a method for preventing an autism spectrum disorder (ASD) in an offspring. In some embodiments, the method comprises administering a vitamin to the mother of the offspring before and/or during pregnancy, wherein the mother has decreased expression and/or DNA methylation of the NHIP gene in a biological sample compared to a control sample.

In another aspect, the disclosure provides a method for preventing or reducing a risk of an offspring for developing an autism spectrum disorder (ASD). In some embodiments, the method comprises:
i) selecting a mother or potential mother of the offspring, wherein the mother or potential mother is selected based on having decreased expression and/or DNA methylation of the NHIP gene in a biological sample compared to a control sample; and
ii) administering a vitamin to the mother or potential mother before and/or during pregnancy, thereby preventing or reducing the risk that the offspring develops an ASD.

In any of the embodiments described herein, the biological sample can be selected from the group consisting of blood, serum, plasma, or saliva from the mother, and placenta, cord blood, blood, saliva and brain from the offspring.

In any of the embodiments described herein, the control sample can be selected from a mother or potential mother having one or more offspring without an ASD or one or more offspring exhibiting typical development.

In another aspect, the disclosure provides a method for preventing or reducing a risk of an offspring for developing an autism spectrum disorder (ASD). In some embodiments, the method comprises administering a therapeutically effective amount of an NHIP gene, an NHIP RNA, or an NHIP peptide, to the mother of the offspring before and/or during pregnancy, thereby preventing or reducing the risk of the offspring for developing an ASD.

In another aspect, the disclosure provides a plasmid or vector comprising the NHIP gene, or DNA encoding an NHIP RNA or peptide. In some embodiments, the plasmid or vector comprises nucleic acid sequences that regulate transcription and/or translation of the NHIP RNA.

In another aspect, the disclosure provides an in vitro method for increasing cell proliferation, the method comprising transfecting a cell with a plasmid or vector of the disclosure.

In another aspect, the disclosure provides a method for regulating gene expression, the method comprising transfecting a cell with a plasmid or vector of the disclosure, and detecting differential expression of one or more genes. In some embodiments, the one or more genes are selected from Table 1 or Table 2.

In another aspect, the disclosure provides an isolated peptide comprising an amino acid sequence having at least about 80% sequence identity to SEQ ID NO:1.

In another aspect, the disclosure provides a fusion protein comprising a peptide of the disclosure.

In another aspect, the disclosure provides a kit comprising reagents for detecting expression of an NHIP RNA or NHIP peptide.

In another aspect, the disclosure provides an array comprising one or more nucleic acid sequences or probes that are capable of hybridizing to an NHIP RNA.

In another aspect, the disclosure provides an array comprising one or more agents that bind to an NHIP peptide immobilized on a solid support. In some embodiments, the one or more agents comprise an antigen binding protein that specifically binds to the NHIP peptide.

In another aspect, the disclosure provides a method for sequencing an NHIP gene sequence. In some embodiments, the method comprises amplifying all or part of an NHIP gene from a biological sample obtained from a subject using a set of primers to produce amplified nucleic acid; and sequencing the amplified nucleic acid. In some embodiments, the subject is a mother or potential mother of an offspring at risk for developing an ASD.

In any of the embodiments described herein, the biological sample can be selected from the group consisting of blood, serum, plasma, or saliva from the mother, and placenta, cord blood, blood, saliva and brain from the offspring.

In any of the embodiments described herein, the control sample can be selected from a mother or potential mother having an offspring (e.g., one or more offspring) without an ASD or an offspring (e.g., one or more offspring) exhibiting typical development.

### BRIEF DESCRIPTION OF THE DRAWINGS

FIGS. 1a-1e. ASD associated DMRs are enriched at fetal brains enhancers and a co-methylated block at 22q13.33 replicated across studies and platforms. (a) Schematic of the experimental design for discovery of ASD DMRs, replication of the co-methylated 22q13.33 locus, genetic associations, and functional follow-up of novel transcript (NHIP). (b) Circular Manhattan plot of the epigenome-wide association of DNA methylation in placenta with ASD diagnosis at 36 months. Results are represented as DMR association test results (−log 10 (p)) ordered by genomics position. Significant thresholds are blue for permutation p-value<0.05, red for FDR adjusted permutation p-value<0.05, and grey for nonsignificant. (c) 134 ASD DMRs (permutation p-value<0.05) tested for enrichment within chromatin states defined by Epigenome Roadmap ChromHMM states 31. Each row represents a different ChromHMM predicted state and each column a single tissue type, with the heatmap plotting the −log 10 (q-value) significance of ASD DMR enrichment. (d) The triangle correlation matrix of methylation levels using the Pearson correlation coefficient for the 12 DMRs located in the 22q13.33 hypomethylated block. (e) The smooth methylation values were averaged over the 22q13.33 hypomethylated block (y-axis) and compared across diagnosis groups (x-axis). In the discovery group, ASD samples had significantly lower methylation than TD samples (MARBLES, HiSeq X, ASD n=46, TD n=46) (p-value=0.003). The same result and direction were observed in the external replication group (EARLI, HiSeq 2500, ASD n=16, TD n=31) (p-value=0.006). For the internal replication group (MARBLES, NovaSeq, ASD n=21, Non-TD n=13, TD n=31), ASD methylation levels were also significantly lower than TD samples (p-value=0.003). Non-TD had lower methylation than TD (p-value=0.048) and higher methylation than ASD at the 22q13 block (p-value=0.049). Comparisons used two-tailed t-test. Box plot center lines, box limits and whiskers represented median, interquartiles range, and minimum and maximum values.

FIGS. 2a-2h. NHIP transcript levels in tissues and cells and in response to hypoxia and evidence for NHIP encoded nuclear peptide. In (a)-(e) RT-qPCR assays, NHIP levels were normalized to GAPDH with at least three independent experiments per condition. (a) NHIP levels in human tissues, including adult brain, fetal brain, placenta, and testis. (b) NHIP levels in placenta samples from the discovery group (ASD n=17, TD n=11). ASD samples show significantly lower NHIP levels than TD samples (two-tailed t-test, p-value=0.005). (c) NHIP levels in human cell lines, HEK293T, IMR90, LUHMES, and SH-SY5Y. In LUHMES cells, NHIP levels were significantly higher in differentiated neurons compared to undifferentiated neurons (two-tailed t-test, p-value=0.020). (d) Differentiated LUHMES cells are more sensitive to hypoxia than undifferentiated LUHMES cells. Formation of reactive oxygen species (ROS) was measured in differentiated and undifferentiated LUHMES cells treated with 100 nM CoCl2, a hypoxia mimetic, or vehicle (mock) (two-tailed t-test, p-value=0.001). (e) NHIP levels increase in response to hypoxia, specifically in differentiated neurons. Differentiated or undifferentiated LUHMES cells were treated with 100 nM CoCl2. In differentiated LUHMES cells, CoCl2 treatment significantly increased NHIP levels (two-tailed t-test, p-value=0.0004). (f) NHIP overexpression in HEK293T cells resulted in a faster doubling time than vector control cells, indicating increased cell proliferation. (g) Vector design of NHIP-peptide-eGFP (dotted line represents excised ATG of EGFP) and combined phase and fluorescent microscopy. Green, eGFP linked to NHIP peptide; red, mCherry, transfection positive control. Scale bars, 100 μm. (h) Immunofluorescent staining of human frontal cortex, showing nuclear localization with anti-NHIP, but not pre-immune control. Blue, DAPI nuclear counterstain; red, anti-NHIP staining. Scale bars, 100 μm. Data are mean±SEM.

FIGS. 3a-3f. A common genetic structural variant is significantly associated with 22q13.33 DNA methylation and ASD. (a) Insertion location (orange) relative to the 22q13.33 hypomethylated block (blue), and the novel transcript, NHIP (red) in the UCSC genome browser. The 22q13.33 co-methylated block was 117,974 bp in length (blue). NHIP TSS was located 7,881 bp downstream from the start site of the 22q13.33 hypomethylated block. The insertion (not in the reference genome) is 15,013 bp upstream from the start site of the 22q13.33 hypomethylated block. (b) The association matrix shows ANOVA p-values for the comparison of the insertion genotype (homozygous for insertion versus not) with smoothed methylation levels within each of 12 DMRs located in 22q13.33 hypomethylated block from discovery group (ASD n=41, TD n=37). (c) Association was tested between insertion genotype (Y, homozygous for insertion; N, not) and 22q13.33 co-methylated block methylation levels (discovery group, ASD n=41, TD n=37). ASD showed significantly lower DNA methylation levels compared to TD placenta samples within the entire 22q13.33 co-methylated block (p-value=0.006). Samples homozygous for the insertion had significantly lower methylation than those not having insertion on one or both alleles (p-value=0.008). When broken down by diagnosis, samples with insertion had significantly lower methylation specifically in ASD samples (p-value=0.003), not TD samples (p-value=0.63). (d) Periconceptional prenatal vitamin use was a significant modifier of 22q13.33 block methylation in placenta (discovery group, ASD n=41, TD n=37). Lower percent methylation at the 22q13.33 co-methylated block was significantly associated with not taking prenatal vitamins during the first month of pregnancy (p-value=0.007), which was in the same direction as ASD risk. (e) UCSC genome browser map shows the insertion location (orange vertical line) relative to two adjacent CTCF sites (green arrows) and NHIP. Both undifferentiated and differentiated LUHMES cells have both CTCF sites, consistent with them being homozygous for the reference sequence. Additional brain tracks show the variability of the upstream CTCF site between human samples. ChromHMM tracks were derived from fetal brain, multiple brain regions, ovary, and placenta. Red, active promoter; yellow, active enhancer; green; active transcriptional elongation; purple, bivalent poised chromatin. (f) Working model to explain ASD risk associated with SV homozygosity. Illustrations created with BioRender.com.

FIGS. 4a-4d. NHIP levels in brain are reduced in ASD and associated with expression of genes enriched for synaptic functions, response to oxidative stress, and ASD risk. (a) Brain samples homozygous for the 22q13.33 insertion had significantly lower NHIP levels compared to those who were not (p-value=0.035). The association NHIP levels and insertion was observed specifically in ASD (p-value=0.024), not in TD (p-value=0.692) (two-tailed t-test, brain, ASD n=13, TD n=10). (b) NHIP-associated differential expression analysis was performed from brain RNA-seq, identifying 851 genome-wide significant genes (FDR adjusted q-value<0.05). (c) Gene ontology (GO) enrichment analysis of the 851 NHIP-associated genes in brain identified significantly enriched terms (FDR adjusted q-value<0.05). Positively associated GO terms are shown in red and negatively associated GO terms are colored in blue. (d) Venn diagram representing the 45 genes in common between NHIP association in brain, differential gene expression (DGE) in NHIP overexpressed cell line, and SFARI ASD risk genes. Genes are listed in Table 1 with common functional categories.

FIG. 5. Full length NHIP was identified in primates, but not other mammals by blat search. NHIP DNA sequence was extracted to blat search against vertebrate databases.

FIG. 6. NHIP transcript levels return to baseline two days following removal of hypoxia mimetic. LUHMES cells were plated in differentiation media at Day 0. At Day 5, differentiated LUHMES cells were treated with CoCl2. After 24 hours of CoCl2 treatment (Day 6), LUHMES cells treated with CoCl2 showed significantly increased NHIP transcript level compared with LUHMES cells with mock treatment (p-value=0.006). CoCl2 treatment were washed and replaced with media at Day 6 to allow LUHMES cells to recover. NHIP transcript level returned to mock control baseline levels after 48 hours of recovery at Day 8 (p-value=0.640).

FIG. 7. HEK293T cells overexpressing NHIP exhibited a significantly altered cell cycle. Overexpression of NHIP in HEK293T cells resulted in a significantly shortened cell cycle doubling time (overexpression cells doubling time=20.23 h, wild type cells doubling time=24.91 h).

FIG. 8. Count matrix of the 1.7 kb insertion genotypes, with a significantly higher frequency in ASD compared to TD (discovery group, ASD n=41, TD n=37) (chi-square test, p-value=0.045).

FIG. 9. Insertion was characterized with PacBio long-read sequencing. QPKN01007947.1 contig 9 mapped to reference genome chr22: 49,381,532-49,466,902 with Miropeat 10 used for visualization. The orange box shows the clear insertion of 1,674 bp in length, comparing the QPKN01007947.1 contig with the reference genome.

FIG. 10. Insertion profiles were validated using PCR genotyping on the same sample. Genotyping primers were designed flanking both sides of the insertion to discriminate the alleles based on size following PCR. Agarose gel electrophoresis and bioanalyzer shown the same result (discovery group, ASD n=41, TD n=37).

FIGS. 11a-11b. When separated by insertion genotype, methylation levels at 22q13.33 were most different between TD and ASD in offspring homozygous for the insertion whose mothers took a prenatal vitamin in month 1 of pregnancy. In other words, for individuals with the genetic risk of the insertion, taking prenatal vitamins at P1 significantly altered 22q13.33 block methylation, in the protective direction (p-value=0.034), a difference which was not significant in those with the insertion whose mothers did not take a prenatal vitamin (p-value=0.400).

FIG. 12. The insertion had significant higher frequency in ASD compared with TD samples in brain. Count matrix was based on the insertion genotypes in brain, showing a higher frequency of individuals with the insertion in ASD compared to TD (ASD n=27, TD n=30) (chi-square test, p-value=0.023).

FIGS. 13a-13b. NHIP overexpression related changes to BRD1 were validated used RT-qPCR. To validate the results from RNA-seq, RT-qPCR was performed on NHIP and BRD1. (a) NHIP was significantly upregulated in the NHIP overexpressing cell line (two-tailed t-test, p-value=1.05E-05). (b) BRD1 was significantly downregulated in the overexpressing cell line (two-tailed t-test, p-value=5.21E-05).

### DETAILED DESCRIPTION

### I. Introduction

Autism spectrum disorders (ASD) are severe neurodevelopmental disorders affecting as many as 1 in 150 children. The present disclosure is based, in part, on the identification of previously uncharacterized ASD risk gene, LOC105373085, renamed NHIP, located in a hypomethylated block at the Chr. 22q13.33 genetic locus (also referred to as the “22q13.33 genetic locus” or “22q13.33 genomic region”). A common structural variant disrupting the proximity of NHIP to a fetal brain enhancer was associated with NHIP expression and methylation levels and ASD risk, demonstrating a common genetic influence. The inventors identified a novel environmentally-responsive ASD risk gene relevant to brain development in a previously under characterized region of the human genome.

### II. Definitions

As used herein, the following terms have the meanings ascribed to them unless specified otherwise.

The articles “a” and “an” can refer to the singular or the plural of a noun modified by the term “a,” for example, one, one or more, or a plurality of the noun.

The terms “autism spectrum disorder,” “autistic spectrum disorder,” “autism” and “ASD” refer to a spectrum of neurodevelopmental disorders characterized by impaired social interaction and communication accompanied by repetitive and stereotyped behavior. Autism includes a spectrum of impaired social interaction and communication, however, the disorder can be roughly categorized into “high functioning autism” or “low functioning autism,” depending on the extent of social interaction and communication impairment. Individuals diagnosed with “high functioning autism” have minimal but identifiable social interaction and communication impairments (i.e., Asperger's syndrome). Additional information on autism spectrum disorders can be found in, for example, Autism Spectrum Disorders: A Research Review for Practitioners, Ozonoff, et al., eds., 2003, American Psychiatric Pub; Gupta, Autistic Spectrum Disorders in Children, 2004, Marcel Dekker Inc; Hollander, Autism Spectrum Disorders, 2003, Marcel Dekker Inc; Handbook of Autism and Developmental Disorders, Volkmar, ed., 2005, John Wiley; Sicile-Kira and Grandin, Autism Spectrum Disorders: The Complete Guide to Understanding Autism, Asperger's Syndrome, Pervasive Developmental Disorder, and Other ASDs, 2004, Perigee Trade; and Duncan, et al., Autism Spectrum Disorders [Two Volumes]: A Handbook for Parents and Professionals, 2007, Praeger.

The terms “typically developing” and “TD” refer to a subject who has not been diagnosed with an autism spectrum disorder (ASD). Typically developing children do not exhibit the ASD-associated impaired communication abilities, impaired social interactions, or repetitive and/or stereotyped behaviors with a severity that is typically associated with a diagnosis of an ASD. While typically developing children may exhibit some behaviors that are displayed by children who have been diagnosed with an ASD, typically developing children do not display the constellation and/or severity of behaviors that supports a diagnosis of an ASD.

The term “isolated,” when applied to a nucleic acid or protein, denotes that the nucleic acid or protein is essentially free of other cellular components with which it is associated in the natural state. It is preferably in a homogeneous state. It can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “polypeptide,” “peptide,” and “protein” are used interchangeably herein to refer to a polymer of amino acid residues, or an assembly of multiple polymers of amino acid residues. The terms apply to amino acid polymers in which one or more amino acid residue is an artificial chemical mimic of a corresponding naturally occurring amino acid, as well as to naturally occurring amino acid polymers and non-naturally occurring amino acid polymer.

The term “sample” refers to any biological specimen obtained from a subject, e.g., a human subject. Samples include, without limitation, whole blood, plasma, serum, red blood cells, white blood cells, saliva, urine, stool, sputum, bronchial lavage fluid, tears, nipple aspirate, breast milk, any other bodily fluid, a tissue sample such as a biopsy of a placenta, and cellular extracts thereof. In some embodiments, the sample is whole blood or a fractional component thereof, such as plasma, serum, or a cell pellet.

The term “subject,” “individual,” or “patient” typically includes humans, but can also include other animals or mammals such as, e.g., other primates, rodents, canines, felines, equines, ovines, porcines, and the like. In some embodiments, the subject is a human subject.

The term “increased risk of developing an ASD” refers to an increased likelihood or probability that a fetus or child having decreased methylation of the chromosome 22q13.33 33 hypomethylated block, or decreased expression and/or decreased methylation of the NHIP gene, will develop symptoms of an ASD in comparison to the risk, likelihood or probability of a fetus or child that does not have decreased methylation of the chromosome 22q13.33 hypomethylated block, or decreased expression and/or decreased methylation of the NHIP gene.

As used herein, the term “administering” includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. One skilled in the art will know of additional methods for administering a therapeutically effective amount of a peptide of the invention for preventing or relieving one or more symptoms associated with the presence or activity of maternal antibodies. By “co-administer” it is meant that a peptide of the invention is administered at the same time, just prior to, or just after the administration of a second drug.

As used herein, the term “treating” refers to any indicia of success in the treatment or amelioration of a pathology or condition, including any objective or subjective parameter such as abatement, remission, diminishing of symptoms or making the pathology or condition more tolerable to the patient, slowing in the rate of degeneration or decline, making the final point of degeneration less debilitating, or improving a patient's physical or mental well-being. The treatment or amelioration of symptoms can be based on objective or subjective parameters, including the results of a physical examination, histopathological examination (e.g., analysis of biopsied tissue), laboratory analysis of urine, saliva, tissue sample, serum, plasma, or blood, or imaging.

The term “gene” refers to a genomic DNA region that contains a specific sequence of nucleotides for transcribing an RNA, including the coding region for a protein and any upstream and downstream sequences that regulate transcription and/or translation of the RNA. The term “NHIP gene” refers to a gene located on chromosome 22 at NC_000022.11, originally referred to as LOC105373085 (see, e.g, www.ncbi.nlm.nih.gov/gene/105373085).

The terms “identical” or “identity,” in the context of two or more polynucleotide or polypeptide or peptide sequences, refer to two or more sequences or subsequences that comprise or consist of the same sequences (i.e., 100 percent identity). Two sequences are “substantially identical” if two sequences have a specified percentage of nucleic acid or amino acid residues that are the same (i.e., 70%, 75%, 80%, 85%, 90%, 95%, 96%, 97%, 98% or 99% sequence identity over a specified region, at least 70%, 75%, 80%, 85%, 90%, 95%, 96%, 97%, 98% or 99% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. With respect to amino acid sequences, identity or substantial identity can exist over a region that is at least 5, 10, 15 or 20 amino acids in length, optionally at least about 25, 30, 35, 40, 50, 75 or 100 amino acids in length, optionally at least about 150, 200 or 250 amino acids in length, or over the full length of the reference sequence. With respect to shorter amino acid sequences, e.g., amino acid sequences of 20 or fewer amino acids, substantial identity exists when one or two amino acid residues are conservatively substituted, according to the conservative substitutions defined herein.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information.

An indication that two polypeptide or peptide sequences are substantially identical occurs when a first polypeptide or peptide is immunologically cross-reactive with the antibodies raised against a second polypeptide or peptide. Thus, a first polypeptide or peptide is typically substantially identical to a second polypeptide or peptide, for example, where the two sequences differ only by conservative substitutions.

Conservative substitution tables providing functionally similar amino acids are well known in the art. For example, substitutions may be made wherein an aliphatic amino acid (e.g., G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, e.g., E or D, may be substituted with its uncharged counterpart, e.g., Q or N, respectively; or vice versa. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
(see, e.g., Creighton, Proteins, 1993).

### III. Detailed Description of the Embodiments

The present disclosure describes methods and compositions for diagnosing or detecting the risk of an autism spectrum disorder (ASD) in offspring including a human child or fetus. The methods and compositions are useful for diagnosing or detecting the risk of ASD by determining methylation levels of genomic loci in tissues from the offspring, mother or potential mother of the offspring. For example, a decrease in methylation levels at certain genomic loci in placental tissues can be used to diagnose a child or fetus as having increased risk for developing ASD.

In some aspects, the methods and compositions are useful for diagnosing or detecting the risk of ASD by determining the methylation status of the Chr. 22q13.33 genomic locus in a biological sample from offspring, mothers or potential mothers of offspring. In some aspects, the methods and compositions are useful for diagnosing or detecting the risk of ASD by determining the expression of the neuronal hypoxia inducible, placenta associated (NHIP) gene in a biological sample from offspring, mothers or potential mothers of offspring. In some embodiments, the methods and compositions are useful for diagnosing or detecting the risk of ASD by determining both the methylation status of the Chr. 22q13.33 genomic locus and the expression of the NHIP gene in a biological sample from offspring, mothers or potential mothers of offspring.

In some embodiments, the offspring is a child (e.g., a neonate). In some embodiments, the offspring is a fetus.

### Patients Subject to Diagnosis

The methods described herein can be performed on any mammal, for example, a human, a non-human primate, a laboratory mammal (e.g., a mouse, a rat, a rabbit, a hamster), a domestic mammal (e.g., a cat, a dog), or an agricultural mammal (e.g., bovine, ovine, porcine, equine). In some embodiments, the patient is a woman and a human.

Any woman capable of bearing a child can benefit from the methods described herein. The child may or may not be conceived, i.e., the woman can be but need not be pregnant. In some embodiments, the woman has a child who is a neonate. In some embodiments, the woman is of childbearing age, i.e., she has begun to menstruate and has not reached menopause

In some embodiments, the methods described herein are performed on a woman carrying a fetus (i.e., who is pregnant). The methods can be performed at any time during pregnancy. In some embodiments, the methods are performed on a woman carrying a fetus whose brain has begun to develop. For example, the fetus may at be at about 12 weeks of gestation or later. In some embodiments, the woman subject to treatment or diagnosis is in the second or third trimester of pregnancy. In some embodiments, the woman subject to treatment or diagnosis is in the first trimester of pregnancy. In some embodiments, the woman is post-partum, e.g., within 6 month of giving birth. In some embodiments, the woman is post-partum and breastfeeding.

Women who will benefit from the present methods may but need not have a familial history of an ASD or an autoimmune disease. For example, the woman may have an ASD or have a family member (e.g., a parent, a child, a grandparent) with an ASD. In some embodiments, the woman suffers from an autoimmune disease or has a family member (e.g., a parent, a child, a grandparent) who suffers from an autoimmune disease.

In some embodiments, the methods described herein comprise the step of determining that the diagnosis is appropriate for the patient, e.g., based on prior medical history or familial medical history or pregnancy status or any other relevant criteria.

### Diagnostic Criteria for Autism Spectrum Disorder

The American Psychiatric Association's Diagnostic and Statistical Manual, Fifth Edition (DSM-5) provides standardized criteria to help diagnose ASD (code 299.00) (see American Psychiatric Association. Diagnostic and statistical manual of mental disorders. 5th ed. Arlington, VA: American Psychiatric Association; 2013.) To meet diagnostic criteria for ASD according to DSM-5, a child must have persistent deficits in each of three areas of social communication and interaction (see A.1. through A.3. below) plus at least two of four types of restricted, repetitive behaviors (see B.1. through B.4. below).

A. Persistent deficits in social communication and social interaction across multiple contexts, as manifested by the following, currently or by history (examples are illustrative, not exhaustive; see text):
1. Deficits in social-emotional reciprocity, ranging, for example, from abnormal social approach and failure of normal back-and-forth conversation; to reduced sharing of interests, emotions, or affect; to failure to initiate or respond to social interactions.
2. Deficits in nonverbal communicative behaviors used for social interaction, ranging, for example, from poorly integrated verbal and nonverbal communication; to abnormalities in eye contact and body language or deficits in understanding and use of gestures; to a total lack of facial expressions and nonverbal communication.
3. Deficits in developing, maintaining, and understand relationships, ranging, for example, from difficulties adjusting behavior to suit various social contexts; to difficulties in sharing imaginative play or in making friends; to absence of interest in peers.

Severity is based on social communication impairments and restricted, repetitive patterns of behavior.

B. Restricted, repetitive patterns of behavior, interests, or activities, as manifested by at least two of the following, currently or by history (examples are illustrative, not exhaustive; see text):
1. Stereotyped or repetitive motor movements, use of objects, or speech (e.g., simple motor stereotypes, lining up toys or flipping objects, echolalia, idiosyncratic phrases).
2. Insistence on sameness, inflexible adherence to routines, or ritualized patterns of verbal or nonverbal behavior (e.g., extreme distress at small changes, difficulties with transitions, rigid thinking patterns, greeting rituals, need to take same route or eat same food every day).
3. Highly restricted, fixated interests that are abnormal in intensity or focus (e.g., strong attachment to or preoccupation with unusual objects, excessively circumscribed or perseverative interests).
4. Hyper- or hyporeactivity to sensory input or unusual interest in sensory aspects of the environment (e.g. apparent indifference to pain/temperature, adverse response to specific sounds or textures, excessive smelling or touching of objects, visual fascination with lights or movement).

Severity is based on social communication impairments and restricted, repetitive patterns of behavior.

C. Symptoms must be present in the early developmental period (but may not become fully manifest until social demands exceed limited capacities, or may be masked by learned strategies in later life).

D. Symptoms cause clinically significant impairment in social, occupational, or other important areas of current functioning.

E. These disturbances are not better explained by intellectual disability (intellectual developmental disorder) or global developmental delay. Intellectual disability and autism spectrum disorder frequently co-occur; to make comorbid diagnoses of autism spectrum disorder and intellectual disability, social communication should be below that expected for general developmental level.

Thus, in some embodiments, the method comprises clinically assessing a child's development by trained, professional examiners using standardized instruments including the Autism Diagnostic Observation Schedule (ADOS) (ref. 71), Autism Diagnostic Interview-Revised (ADI-R) (ref 72), and Mullen Scales of Early Learning (MSEL) (ref. 73). Based on a previously published algorithm, children were classified into three outcome groups: ASD, TD and Non-TD (refs. 43,74,75). Children with ASD had scores over the ADOS cutoff and fit ASD DSM-5 criteria (see above). Children with TD had all MSEL scores within 2 standard deviations (SD) and no more than one MSEL subscale 1.5 SD below the normative mean together, with scores on the ADOS at least three points lower than the ASD cutoff. Children with Non-TD did not meet ASD or TD criteria, but had elevated ADOS scores and low MSEL scores, defined as two or more MSEL subscales with more than 1.5 SD below normative mean, or at least one MSEL subscale more than 2 SD below normative mean. The above assessment can be based on the age of child, for example, from 12 months through adulthood, language and developmental level.

The Mullen Scales of Early Learning (Mullen, or MSEL; Mullen, 1995) is an individually administered, norm-referenced measure of early intellectual development and school readiness, permitting targeted intervention at a young age. This instrument measuring cognitive functioning was designed to be used with children from birth through 68 months. It consists of a Gross-Motor Scale and four Cognitive Scales: Visual Reception, Fine-Motor, Receptive Language, and Expressive Language. The Gross-Motor Scale is for use with children ages birth through 33 months, whereas the Cognitive Scales are used with children ages birth to 68 months. T-scores (mean of 50 and a standard deviation of 10) are given for individual scales, and an optional Early Learning Composite standard score (mean of 100 and a standard deviation of 15) serves as an overall estimate of cognitive functioning (see the internet at www.txautism.net/evaluations/mullen-scales-of-early-learning). The MSEL score is described in Shank L. (2011) Mullen Scales of Early Learning. In: Kreutzer J. S., DeLuca J., Caplan B. (eds) Encyclopedia of Clinical Neuropsychology. Springer, New York, NY (see the internet at doi.org/10.1007/978-0-387-79948-3_1570), which is incorporated by reference herein.

### Biological Samples

In some embodiments, the biological sample is obtained from the mother or potential mother of the offspring. In some embodiments, the biological sample comprises, but is not limited to blood, serum, plasma, or saliva from the mother or potential mother of the offspring. In some embodiments, the biological sample is obtained from an offspring, and comprises, but is not limited to, placenta, cord blood, blood, saliva or brain from the offspring.

The biological sample can be obtained from the mother during pregnancy or after birth of the offspring. For example, fetal tissues can be obtained from the mother during pregnancy or after birth of the child.

In some embodiments, the biological sample is homozygous for a structural variant insertion upstream of the 22q13.33 locus. In some embodiments, the biological sample is homozygous for a structural variant insertion (chr22: 49029657, hg38) approximately 15 Kb upstream of the 22q13.33 locus.

### Methods for Determining the Risk of an Offspring for Developing ASD

In some embodiments, described herein is a method for determining the risk of an offspring for developing ASD, the method comprising detecting, in a biological sample from the offspring, mother or potential mother of the offspring, the methylation levels over the chromosome 22q13.33 genomic region, wherein decreased methylation levels indicate an increased risk of the offspring for developing an ASD. In some embodiments, the method comprises detecting, in a biological sample from the offspring, mother or potential mother of the offspring, DNA methylation levels of a NHIP gene, wherein decreased methylation levels of the NHIP gene indicates an increased risk of the offspring for developing an ASD. In some embodiments, the method comprises detecting, in a biological sample from the offspring, mother or potential mother of the offspring, both the methylation levels over the chromosome 22q13.33 genomic region and the DNA methylation levels of a NHIP gene, wherein decreased methylation levels over the chromosome 22q13.33 genomic region and the NHIP gene indicates an increased risk of the offspring for developing an ASD.

In some embodiments, the 22q13.33 genomic region is hypomethylated. In some embodiments, the NHIP gene is hypomethylated. In some embodiments, the methylation levels over the chromosome 22q13.33 genomic region, and/or the DNA methylation levels of a NHIP gene, are compared to the respective methylation levels from a control sample or a control value. DNA methylation can be expressed as percent methylation for each sample. In some embodiments, the methylation levels comprise smoothed methylation values averaged over the 22q13.33 genomic region. Methods for determining smoothed methylation values are described in the Examples.

In some embodiments, the method comprises detecting, in a biological sample from the offspring, mother or potential mother of the offspring, expression of a NHIP gene, wherein decreased expression of the NHIP gene indicates an increased risk of the offspring for developing an ASD. In some embodiments, the expression levels of the NHIP gene are compared to the expression levels of the NHIP gene in a control sample. In some embodiments, the method comprises detecting, in a biological sample from the offspring, mother or potential mother of the offspring, both the expression and DNA methylation levels of a NHIP gene, wherein decreased expression and methylation levels of the NHIP gene indicates an increased risk of the offspring for developing an ASD. In some embodiments, both the expression and DNA methylation levels of the NHIP gene are compared to the expression and DNA methylation levels of the NHIP gene in a control sample or a control value.

In some or all of the embodiments described herein, the control sample can comprise a biological sample from an offspring, mother or potential mother of an offspring that does not have an ASD or an offspring exhibiting typical development. For example, the control sample can comprise a biological sample from an offspring that does not have an ASD or an offspring exhibiting typical development based on MSEL scores within 2 standard deviations (SD) and no more than one MSEL subscale 1.5 SD below the normative mean together, with scores on the ADOS at least three points lower than the ASD cutoff, as described above.

In some embodiments, the methylation status of the chromosome 22q13.33 genomic region, or the expression or methylation status of the NHIP gene, is compared to a control or reference value. The control or reference value can be determined by measuring the methylation status of the chromosome 22q13.33 genomic region, or the expression or methylation status of the NHIP gene, in a biological sample from an offspring, mother or potential mother of an offspring that does have an ASD or an offspring exhibiting typical development. In some embodiments, the control or reference value is determined by measuring the methylation status of the chromosome 22q13.33 genomic region, or the expression or methylation status of the NHIP gene, in a biological sample from an offspring that does not have an ASD or an offspring exhibiting typical development based on MSEL scores within 2 standard deviations (SD) and no more than one MSEL subscale 1.5 SD below the normative mean together, with scores on the ADOS at least three points lower than the ASD cutoff, as described above.

Methylation status of the chromosome 22q13.33 genomic region and/or the NHIP gene can be determined, for example, by Whole Genome Bisulfite Sequencing (WGBS) or by DNA methylation array analysis. Specific methods for determining the methylation status of the chromosome 22q13.33 genomic region are described in the Examples.

Expression of the NHIP gene can be determined by detecting expression of an RNA or peptide expressed by the gene. RNA can be detected, for example, by amplifying the RNA, quantifying the RNA, or sequencing the RNA. Specific example for detecting RNA include reverse transcription of the mRNA followed by first strand cDNA synthesis and amplification by PCR (RT-PCR), Northern analysis, TaqMan PCR assays, or sequencing the RNA (RNA-seq). In some embodiments, the RNA is transcribed from an open reading frame comprising the DNA sequence:

(SEQ ID NO: 2)
ATGGTGAGAGGAGAGGCCACCGCACGAACGGAAGAAGCGATGGAGACGG
TCTTTACGACC.

Expression of an NHIP peptide encoded by an RNA transcribed from the NHIP gene can be detected, for example, by contacting the peptide with an antibody that binds to the peptide, and detecting binding between the antibody and the peptide. Examples for detecting binding between the antibody and peptide include Western analysis, detecting a label conjugated to the antibody, binding a labeled secondary antibody to the anti-NHIP peptide antibody, or by immunostaining of tissues with the antibody.

Secondary antibodies can be labeled with any directly or indirectly detectable moiety, including a fluorophore (e.g., fluoroscein, phycoerythrin, quantum dot, Luminex bead, fluorescent bead), an enzyme (e.g., peroxidase, alkaline phosphatase), a radioisotope (e.g., ³H, ³²P, ¹²⁵I), or a chemiluminescent moiety. Labeling signals can be amplified using a complex of biotin and a biotin binding moiety (e.g., avidin, streptavidin, neutravidin). Fluorescently labeled anti-human IgG antibodies are commercially available from Molecular Probes, Eugene, OR. Enzyme-labeled anti-human IgG antibodies are commercially available from Sigma-Aldrich, St. Louis, MO and Chemicon, Temecula, CA.

In some embodiments, the peptide is detected by linking the peptide to a detectable label. Examples of detectable labels include but are not limited to biotin/strepavidin, a fluorescent label, a chemiluminescent label, or a radioactive label. In some embodiments, the label is covalently attached to the peptide. Expression of the NHIP peptide can also be detected by mass spectrometry (e.g., LC/MS-MS).

In some embodiments, the NHIP peptide comprises the amino acid sequence

(SEQ ID NO: 1)
MVRGEATARTEEAMETVFTT.

### Methods for Preventing ASD in an Offspring

The disclosure also provides methods for preventing an autism spectrum disorder (ASD) in an offspring. In some embodiments, the method comprises administering a vitamin to the mother of the offspring before and/or during pregnancy. In some embodiments, the vitamin comprises a, one or more, or a plurality of dietary methyl group(s).

In some embodiments, the offspring and/or mother has decreased methylation levels over the chromosome 22q13.33 genomic region in a biological sample compared to a control sample or control value. In some embodiments, the offspring and/or mother has decreased methylation levels of the NHIP gene in a biological sample compared to a control sample or control value. In some embodiments, the offspring and/or mother has decreased expression the NHIP gene in a biological sample compared to a control sample or control value. In some embodiments, the mother is homozygous for a structural variant inserted upstream of the chr22q13.33 genomic region. In some embodiments, the structural variant is inserted about 15 Kb upstream of the chr22q13.33 genomic region.

In some embodiments, a vitamin for use as a medicament in preventing ASD in an offspring is provided. In some embodiments, a vitamin for use in preventing ASD in an offspring is provided. In some embodiments, the offspring and/or mother has decreased methylation levels over the chromosome 22q13.33 genomic region in a biological sample compared to a control sample or control value. In some embodiments, the offspring and/or mother has decreased methylation levels of the NHIP gene in a biological sample compared to a control sample or control value. In some embodiments, the offspring and/or mother has decreased expression the NHIP gene in a biological sample compared to a control sample or control value. In some embodiments, the mother is homozygous for a structural variant inserted upstream of the chr22q13.33 genomic region. In some embodiments, the structural variant is inserted about 15 Kb upstream of the chr22q13.33 genomic region. In some embodiments, the vitamin comprises a, one or more, or a plurality of dietary methyl group(s).

### Methods for Preventing or Reducing a Risk of an Offspring for Developing ASD

The disclosure also provides methods for preventing or reducing a risk of an offspring for developing an autism spectrum disorder (ASD). In some embodiments, the method comprises selecting a mother or potential mother of the offspring, wherein the mother or potential mother is selected based on having decreased methylation levels over the chromosome 22q13.33 genomic region in a biological sample compared to a control sample or control value. In some embodiments, the mother or potential mother is selected based on having decreased methylation levels of the NHIP gene in a biological sample compared to a control sample or control value. In some embodiments, the mother or potential mother is selected based on having decreased expression of the NHIP gene in a biological sample compared to a control sample or control value.

In some embodiments, the method further comprises administering a treatment to the mother or potential mother before and/or during pregnancy. In some embodiments, the treatment comprises administering a therapeutically effective amount of a therapeutic agent that is sufficient to prevent or reduce the risk that the offspring develops an ASD. In some embodiments, the treatment comprises administering a vitamin to the mother or potential mother before and/or during pregnancy. In some embodiments, the vitamin comprises a, one or more, or a plurality of dietary methyl group(s).

In some embodiments, the method for preventing or reducing a risk of an offspring for developing an ASD comprises administering a therapeutically effective amount of an NHIP gene, an NHIP RNA, or an NHIP peptide, to the mother of the offspring before and/or during pregnancy, thereby preventing or reducing the risk of the offspring for developing an ASD.

In some embodiments, an NHIP gene, an NHIP RNA, or an NHIP peptide for use as a medicament in preventing or reducing a risk of an offspring for developing an ASD is provided. In some embodiments, an NHIP gene, an NHIP RNA, or an NHIP peptide for use in preventing or reducing a risk of an offspring for developing an ASD is provided. In some embodiments, the use comprises selecting a mother or potential mother of the offspring, wherein the mother or potential mother is selected based on having decreased methylation levels over the chromosome 22q13.33 genomic region in a biological sample compared to a control sample or control value. In some embodiments, the mother or potential mother is selected based on having decreased methylation levels of the NHIP gene in a biological sample compared to a control sample or control value. In some embodiments, the mother or potential mother is selected based on having decreased expression of the NHIP gene in a biological sample compared to a control sample or control value.

### Methods of Treatment

The disclosure also provides methods for treating an offspring. In some embodiments, the method comprises administering a therapeutically effective amount of a therapeutic agent to the mother of the offspring before and/or during pregnancy. In some embodiments, the therapeutic agent is a vitamin, and the method comprises administering a therapeutically effective amount of a vitamin to the mother of the offspring before and/or during pregnancy. In some embodiments, the vitamin comprises a, one or more, or a plurality of dietary methyl group(s).

In some embodiments, the offspring is from a mother who has a family history of an ASD or an autoimmune disease. For example, the woman may have an ASD or have a family member (e.g., a parent, a child, a grandparent) with an ASD. In some embodiments, the woman suffers from an autoimmune disease or has a family member (e.g., a parent, a child, a grandparent) who suffers from an autoimmune disease. In some embodiments, the mother is homozygous for a structural variant inserted upstream of the chr22q13.33 genomic region. In some embodiments, the structural variant is inserted about 15 Kb upstream of the chr22q13.33 genomic region.

In some embodiments, the methods for treating an offspring can be performed at any time during pregnancy. In some embodiments, the methods for treating an offspring are performed on a woman carrying a fetus whose brain has begun to develop. For example, the fetus may at be at about 12 weeks of gestation or later. In some embodiments, the methods for treating an offspring are performed on a woman in the second or third trimester of pregnancy. In some embodiments, the methods for treating an offspring are performed on a woman in the first trimester of pregnancy.

In some embodiments, a vitamin for use as a medicament to treat an offspring is provided. In some embodiments, a vitamin for use in the treatment of ASD in an offspring is provided. In some embodiments, the offspring is from a mother who has a family history of an ASD or an autoimmune disease. For example, the woman may have an ASD or have a family member (e.g., a parent, a child, a grandparent) with an ASD. In some embodiments, the woman suffers from an autoimmune disease or has a family member (e.g., a parent, a child, a grandparent) who suffers from an autoimmune disease. In some embodiments, the mother is homozygous for a structural variant inserted upstream of the chr22q13.33 genomic region. In some embodiments, the structural variant is inserted about 15 Kb upstream of the chr22q13.33 genomic region. In some embodiments, the vitamin comprises a, one or more, or a plurality of dietary methyl group(s).

### Methods for Sequencing an NHIP Gene

The disclosure also provides methods for sequencing an NHIP gene. In some embodiments, the method comprises amplifying all or part of an NHIP gene from a biological sample obtained from a subject using a set of primers to produce amplified nucleic acid, and sequencing the amplified nucleic acid. In some embodiments, the method comprises sequencing RNA transcribed from or expressed by the NHIP gene. In some embodiments, the method comprises reverse-transcribing mRNA to cDNA molecules, and amplifying the cDNA molecules to produce a library of cDNA, and sequencing the library (often referred to as RNA-seq).

### Methods for Detecting an NHIP Peptide

The disclosure also provides methods for detecting an NHIP peptide in a subject. In some embodiments, the method comprises obtaining a biological sample from the subject, and detecting the presence of the NHIP peptide in the subject. In some embodiments, the NHIP peptide is detected by contacting the biological sample with an anti-NHIP antibody and detecting binding between the NHIP peptide and the antibody. In some embodiments, the NHIP peptide is detected by performing mass spectrometry on peptides isolated from the biological sample.

In some embodiments, the subject is an offspring at risk for developing an ASD. In some embodiments, the subject is a mother or potential mother of an offspring at risk for developing an ASD.

### Plasmids and Vectors

The disclosure also provides compositions that are useful for diagnosing, preventing or treating an ASD. In some embodiments, the composition is a plasmid comprising polynucleotide sequences comprising the NHIP gene. In some embodiments, the plasmid comprises DNA sequences encoding an NHIP RNA or NHIP peptide.

In some embodiments, the composition is a vector comprising polynucleotide sequences comprising the NHIP gene. In some embodiments, the vector comprises DNA sequences encoding an NHIP RNA or NHIP peptide.

In some embodiments, the plasmid or vector further comprises nucleic acid sequences that regulate transcription and/or translation of the NHIP RNA. In some embodiments, the vector is an expression vector comprising sequences that regulate transcription and/or translation in mammalian cells.

In some embodiments, the plasmid or vector comprises the nucleotide sequence of SEQ ID NO:2, SEQ ID NO:4, or SEQ ID NO:5.

### Methods for Increasing Cell Proliferation

The disclosure also provides methods for increasing cell proliferation. In some embodiments, the method is an in vitro method. In some embodiments, the method comprises transfecting a cell with a plasmid or vector described herein, and determining the rate of cell proliferation in the transfected cells.

### Methods for Regulating Gene Expression

The disclosure also provides methods for regulating gene expression. In some embodiments, the method comprises transfecting a cell with a plasmid or vector described herein, and detecting differential expression of one or more genes. Differential expression can be detected, for example, by DESeq2 as described in the Examples. Differential expression can also be determined by Limma-Voom. Limma is an R package that was originally developed for differential expression (DE) analysis of microarray data, and Voom is a function in the limma package that modifies RNA-Seq data for use with limma. See the internet at ucdavis-bioinformatics-training.github.io/2018-June-RNA-Seq-Workshop/thursday/DE.html. Differential expression can also be determined by Bioconductor package edgeR for differential expression analyses of read counts arising from RNA-Seq. See the internet at bioconductor.org/packages/release/bioc/vignettes/edgeR/inst/doc/edgeRUsersGuide.pdf.

### Peptides

The present disclosure also provides isolated peptides expressed by an NHIP gene. In some embodiments, the peptide is translated from an RNA expressed by the NHIP gene. In some embodiments, the peptide is translated from an open reading frame comprising the DNA sequence

(SEQ ID NO: 2)
ATGGTGAGAGGAGAGGCCACCGCACGAACGGAAGAAGCGATGGAGACGG
TCTTTACGACC.

The peptides described herein can be produced by any suitable means known or later discovered in the field, e.g., synthesized in vitro, purified or substantially purified from a natural source, or recombinantly produced from eukaryotic or prokaryotic cells. In some embodiments, the peptide can be isolated from endogenous tissues or cells obtained from a biological sample, or from cells or tissues in vitro.

In some embodiments, the NHIP peptide comprises or consists of the amino acid sequence MVRGEATARTEEAMETVFTT (SEQ ID NO:1). In some embodiments, the peptide is substantially identical to the amino acid sequence MVRGEATARTEEAMETVFTT (SEQ ID NO:1) (i.e., at least 70%, 75%, 80%, 85%, 90%, 95%, 96%, 97%, 98% or 99% sequence identity to SEQ ID NO:1). In some embodiments, the peptide comprises one or more amino acid residues that are conservatively substituted, according to the conservative substitutions defined herein.

### Fusion Proteins

The present disclosure also provides fusion proteins comprising an NHIP peptide described herein. In some embodiments, the fusion protein comprises an NHIP peptide described herein linked to a fusion partner polypeptide. In some embodiments, the fusion protein comprises an NHIP peptide described herein covalently linked to a fusion partner polypeptide. In some embodiments, the fusion partner comprises a detectable polypeptide, such as green fluorescent protein (GFP), enhanced GFP (eGFP), or mCherry. In some embodiments, the fusion protein comprises an NHIP peptide described herein linked to an amino acid sequence tag. In some embodiments, the tag is an epitope tag, an affinity tag, or a fluorescent tag.

In some embodiments, the fusion protein is produced from a plasmid or expression vector comprising nucleotide sequences that encode the peptide and fusion partner. In some embodiments, the plasmid or expression vector further comprises regulatory sequences that control transcription and/or translation of the nucleotide sequences that encode the peptide and fusion partner or the peptide and an amino acid sequence tag.

### Kits

The present disclosure also provides kits for determining whether an offspring such as a fetus or child is at an increased risk of developing an autism spectrum disorder (ASD). Relatedly, the kits can also be used to determine whether a mother or potential mother is at an increased risk of bearing a child who will develop an ASD.

Materials and reagents to carry out the methods described herein can be provided in kits to facilitate execution of the methods. As used herein, the term “kit” includes a combination of articles that facilitates a process, assay, analysis, or manipulation. In particular, kits comprising the compositions described herein find utility in a wide range of applications including, for example, diagnostics, prognostics, and method of treatment.

Kits can contain chemical reagents as well as other components. In addition, the kits described herein can include, without limitation, instructions to the kit user, apparatus and reagents for sample collection and/or purification, apparatus and reagents for product collection and/or purification, reagents for bacterial cell transformation, reagents for eukaryotic cell transfection, previously transformed or transfected host cells, sample tubes, holders, trays, racks, dishes, plates, solutions, buffers or other chemical reagents, suitable samples to be used for standardization, normalization, and/or control samples. Kits described herein can also be packaged for convenient storage and safe shipping, for example, in a box having a lid.

In some embodiments, the kits also comprise labeled secondary antibodies used to detect binding of an antibody to an NHIP peptide. The secondary antibodies bind to the constant or “C” regions of different classes or isotypes of immunoglobulins IgM, IgD, IgG, IgA, and IgE. Usually, a secondary antibody against an IgG constant region is included in the kits, such as, e.g., secondary antibodies against one of the IgG subclasses (e.g., IgG1, IgG2, IgG3, and IgG4). Secondary antibodies can be labeled with any directly or indirectly detectable moiety, including a fluorophore (e.g., fluoroscein, phycoerythrin, quantum dot, Luminex bead, fluorescent bead), an enzyme (e.g., peroxidase, alkaline phosphatase), a radioisotope (e.g., ³H, ³²P, ¹²⁵I), or a chemiluminescent moiety. Labeling signals can be amplified using a complex of biotin and a biotin binding moiety (e.g., avidin, streptavidin, neutravidin). Fluorescently labeled anti-human IgG antibodies are commercially available from Molecular Probes, Eugene, OR. Enzyme-labeled anti-human IgG antibodies are commercially available from Sigma-Aldrich, St. Louis, MO and Chemicon, Temecula, CA.

In some embodiments, the kit comprises reagents for detecting expression of an NHIP RNA or NHIP peptide. Examples of reagents for detecting expression of an NHIP RNA include one or more primers for reverse transcribing and/or amplifying the RNA, a reverse transcriptase, and a polymerase. Examples of reagents for detecting expression of an NHIP peptide include anti-NHIP peptide antibodies, and labeled secondary antibodies as described herein.

In some embodiments, the kit comprises an NHIP peptide attached to a solid support. In some embodiments, the solid support is a multiwell plate, an ELISA plate, a microarray, a chip, a bead, a porous strip, or a nitrocellulose filter. In some embodiments, the peptide or plurality thereof is immobilized on (e.g., covalently attached to) the solid support.

### Arrays

The present disclosure also provides arrays. In some embodiments, the array comprises one or more nucleic acid sequences or probes that are capable of hybridizing to an NHIP RNA.

In some embodiments, the array comprises one or more agents that bind to an NHIP peptide immobilized on a solid support. In some embodiments, the one or more agents comprise an antigen binding protein that specifically binds to the NHIP peptide.

### Reaction Mixtures

The present disclosure also provides reaction mixtures for amplifying the NHIP gene DNA or RNA. In some embodiments, the reaction mixture comprises primers for amplifying the NHIP gene DNA or RNA, a polynucleotide template comprising sequences from the NHIP gene, and free nucleotides. In some embodiments, the polynucleotide template comprises the nucleic acid sequence ATGGTGAGAGGAGAGGCCACCGCACGAACGGAAGAAGCGATGGAGACGGTCTT TACGACC (SEQ ID NO:2), or a complement thereof.

### EXAMPLES

The following examples are offered to illustrate, but not to limit the claimed invention.

### Example 1. Association of ASD Risk with Placental DNA Methylation in Two High-Risk Familial ASD Cohorts

This example describes a study that was performed to determine the association between placental DNA methylation and ASD risk.

### Introduction

Autism spectrum disorders (ASD) are growing in prevalence, with 1 in 54 children diagnosed in the United States¹. Diagnosis of ASD is based on a child's behavioral difficulties in social communication and interactions, languages deficits, restricted interests and repetitive behaviors, and sensory sensitives. The etiology of ASD is complex and heterogeneous, and is likely to involve multiple genetic and environmental factors, as well as poorly understood gene-environment interactions²⁻⁴. Twin and sibling studies have shown a strong heritability of ASD risk within families, and most genetic risk for ASD is expected to come from common variants⁵. Exome sequencing of ASD trios has identified genes mutated in rare genetic ASD children, which are enriched for neuronal, embryonic development and chromatin regulation functions, but no single gene explains more than 1% of disease risk^{6,7}. A large genome-wide association study (GWAS) calculated that an individual's ASD risk depends on the level of polygenic burden from thousands of common variants in a dose-dependent manner⁸. ASD genetic susceptibility predictions can be improved by adding single nucleotide polymorphism (SNP) weights using polygenic risk scores (PRS) from ASD-correlated traits, including schizophrenia, depression, and educational attainment⁸⁻¹⁰. Common polygenic risk may also interplay with early environmental and perinatal factors. For example, a schizophrenia PRS was shown to be more than five times greater in the presence of early-life maternal complications¹¹. In addition, the SZ-PRS differences corresponded with placental gene expression, consistent with the importance of placental gene regulation as a window into neurodevelopment.^{11,12} However, most ASD genetic or environmental studies have not included placental molecular measures, despite the potential convergence between placental biology and genetic risk for ASD. Term placenta is an accessible tissue normally discarded at birth, however the convergence between placental biology and genetic risk for ASD is relatively unexplored.

Placenta maintains a distinct landscape of DNA methylation characterized by partially methylated domains (PMDs), which is more similar to oocytes and preimplantation embryos than fetal or adult tissues¹³⁻¹⁵. Because of its multiple roles in support of fetal development during intrauterine life, the placenta is a promising tissue for identifying DNA methylation alterations at genes relevant to fetal brain and gene-environment interactions in ASD¹⁶⁻¹⁹. Most epigenome-wide association studies (EWAS) for ASD have used array-based methods to assess DNA methylation which lack coverage over the most epigenetically and genetically polymorphic regions of the human genome, such as correlated regions of systemic interindividual variation (CORSIVs) and structural variants (SVs)²⁰. CORSIVs are sensitive to periconceptional environment, observed across diverse tissues, associated with human disease genes, and are enriched for transposable elements and subtelomeric locations^{20,21}. SVs arising from transposable elements have been associated with many human phenotypes, especially immune response, and neuropsychiatric disorders, such as schizophrenia²²⁻²⁴. SVs exhibit a nonrandom distribution in hotspots within relatively gene-poor regions in primate genomes, but are enriched for gene functions in oxygen transport, sensory perception, synapse assembly, and antigen-binding^{25,26}. Recent studies suggested that a large SV burden was associated with lower cognitive ability²⁷⁻²⁹ and ASD³⁰, but most GWAS and EWAS studies ignore SVs and CoRSIVs in the genome. Therefore, the combination of utilizing the unique placental DNA methylation landscape reflective of in utero gene expression with sequencing-based epigenome-wide investigations inclusive of understudied genomic regions is warranted.

Here, we investigated the association of ASD risk with placental DNA methylation in two high-risk familial ASD cohorts through whole genome bisulfite sequencing (WGBS) analysis of 204 individuals. We identified a block of differential methylation in ASD at 22q13.33, a region previously described as a CORSIV and SV hotspot but not previously associated with ASD. A novel gene LOC105373085 (renamed as NHIP for neuronal hypoxia inducible, placenta associated) within 22q13.33 was demonstrated to be expressed in brain, responsive to oxidative stress, and to influence expression of other known ASD-risk genes. A common SV insertion within 22q13.33 was significantly associated with increased ASD risk, reduced expression of NHIP, and reduced methylation, but first month prenatal vitamin use counteracted this effect. Together, these results demonstrate a novel ASD risk gene regulatory locus at the interface of common genetics and perinatal environmental resilience.

### Results

### Differential Methylation Analysis Using WGBS Identifies a Hypomethylated Block at 22q13.33 in ASD Placenta

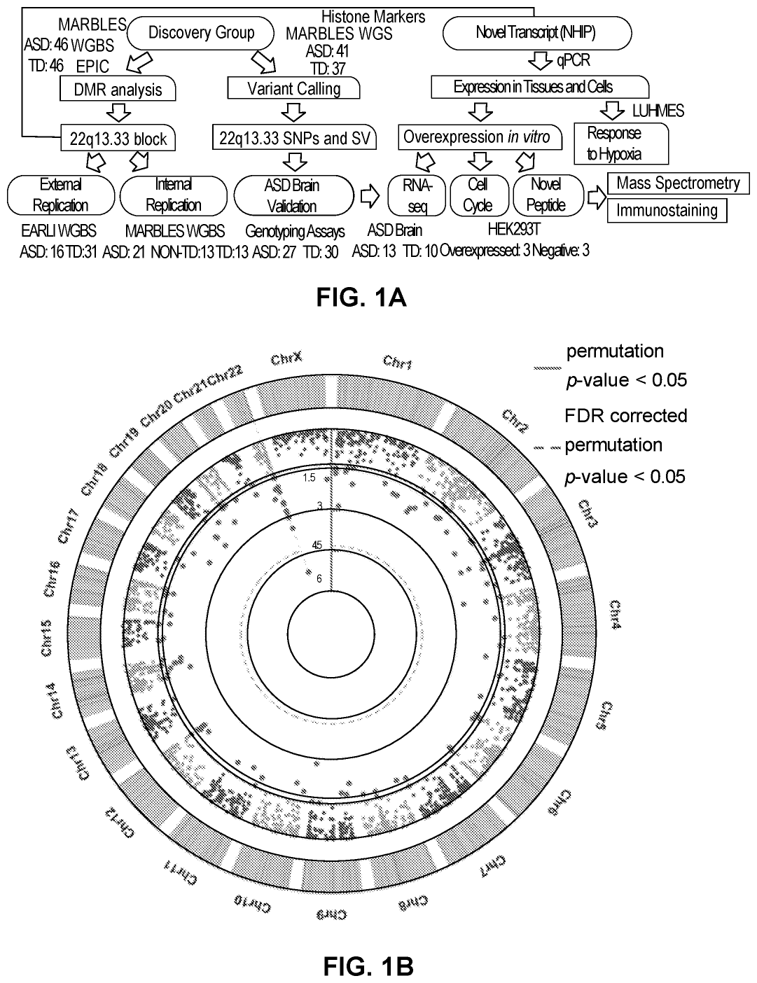

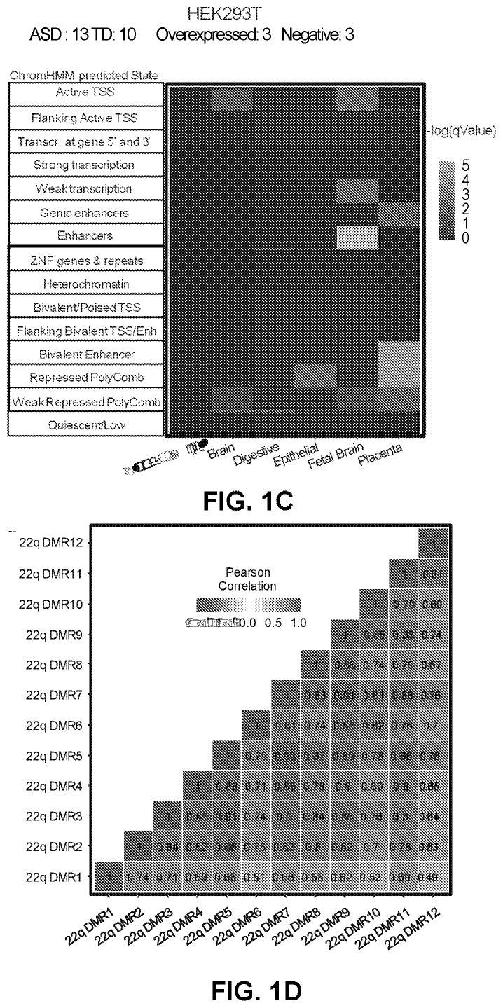

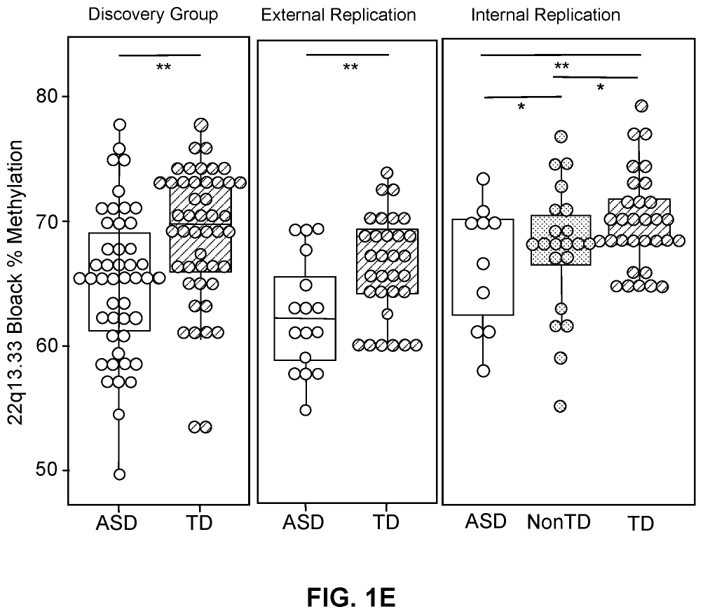

To identify novel regions of epigenetic alterations in placenta discriminating later ASD diagnosis, we performed WGBS analysis of genome-wide DNA methylation on 204 subjects from two prospective high-risk ASD cohorts (MARBLES and EARLI) with a diagnosis outcome at 36 months (FIG. 1a). No demographic or technical variables were significantly associated with ASD outcome, but scores related to ASD severity and cognition were associated, as expected. Global methylation levels over 20 kb windows were also not significantly different by diagnostic group. Discovery, external replication, and internal replication groups were analyzed separately, since sequencing platform differences impacted global methylation levels.

Differentially methylated regions (DMRs) distinguishing ASD from typical development (TD) placental samples were identified with a permutation-based statistical approach, adjusted for sex and placental cell types, to identify broad epigenomic signatures of multiple gene regulatory regions at a genome-wide level in the discovery group. 134 DMRs (permutation p-value<0.05) representing an average size of 1027 bp with 5-10% smoothed methylation differences, including 77 hyper- and 57 hypo-methylated in ASD compared to TD, mapped to 183 genes (FIG. 1b). A cluster of 12 ASD DMRs mapped to 22q13.33, all hypomethylated in ASD (5-7% difference from TD), including one that also passed genome-wide significance (FDR adjusted p-value<0.05). Methylation levels within the 134 ASD DMRs were specifically associated with autism severity and cognitive scores, but not other demographic and technical variables. Further evidence that DMRs identified in placenta reflect epigenetic differences relevant to brain and development came from the significant enrichment of ASD DMRs in fetal brain enhancers, as well as bivalent enhancer and repressed polycomb regions of placenta compared to background regions using ChromHMM-defined chromatin states from the Roadmap Epigenomics Project³¹ (FIG. 1c). Demonstrating their functional relevance, hyper-methylated ASD DMRs were enriched within 0-5 kb and 5-50 kb windows downstream of transcription start sites (TSS), at CpG islands and shores for both hyper- and hypo-methylated DMRs, and at known transcription factor binding sites. Genes mapping to placental ASD DMRs significantly overlapped with ASD risk genes from the Simons Foundation Autism Research Initiative (SFARI) dataset³². The overrepresentation of 12 DMRs at 22q13.33 hypomethylated in ASD drove the additional enrichment at >500 kb of TSS as well as gene ontology (GO) enrichment for functions in histone acetyltransferase (HATs) and chromatin modification, due to the assignment of the 22q13.33 hypomethylated DMRs to the nearest downstream gene BRD1, a histone acetyltransferase. Based on these results, we decided to focus subsequent analyses on further understanding the impact of the 22q13.33 hypomethylated locus on ASD risk.

The 22q13.33 DMRs hypomethylated in ASD were highly positively correlated with each other and formed a 118 kb hypomethylation cluster that was also detected as a hypomethylated block (chr22: 49044669-49162642, hg38) (FIG. 1d), which was also previously described as a CORSIV²⁰. We therefore examined smoothed methylation levels over the 118 kb 22q13.33 block for replication in a different ASD enriched risk cohort (EARLI, external replication group). Similar to the discovery group, 22q13.33 block methylation levels were significantly lower in ASD compared to TD (FIG. 1e). Furthermore, an independent “internal replication group” of MARBLES subjects using a different sequencing platform also showed significantly lower 22q13.33 DNA methylation levels in ASD compared to either TD or the additional diagnostic Non-TD samples, defined as atypical cognitive scores but not ASD (FIG. 1e). These results demonstrate that hypomethylation over the 118 kb 22q13.33 co-methylated block is a reproducible finding across different cohorts and platforms, specifically distinguishing placental samples of newborns later diagnosed with ASD.

NHIP is a Primate-Specific Gene Dynamically Expressed During Neuronal Differentiation that Exhibits Reduced Expression in ASD

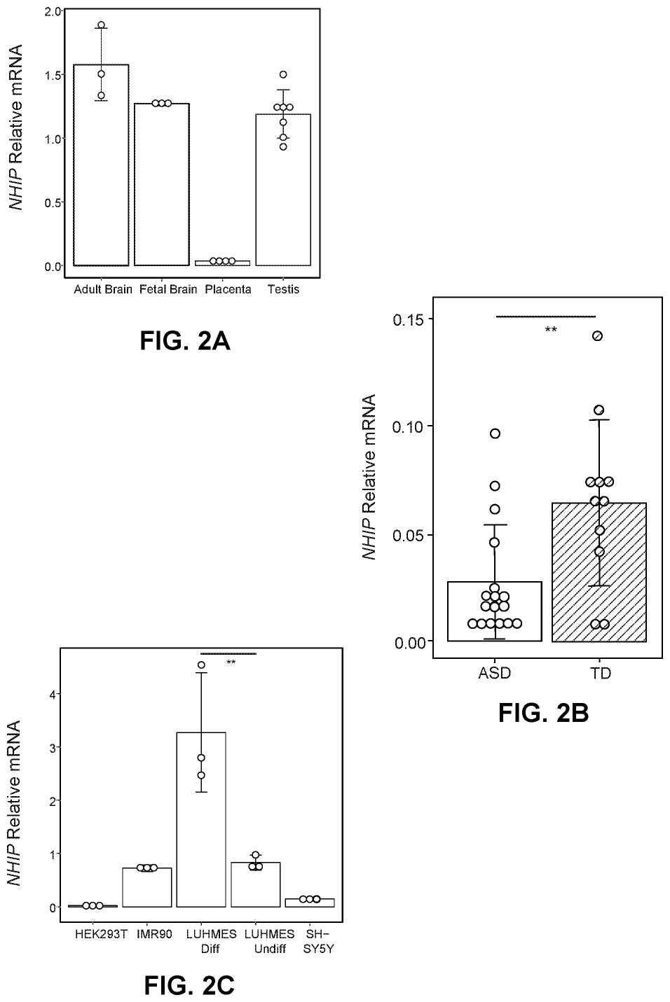

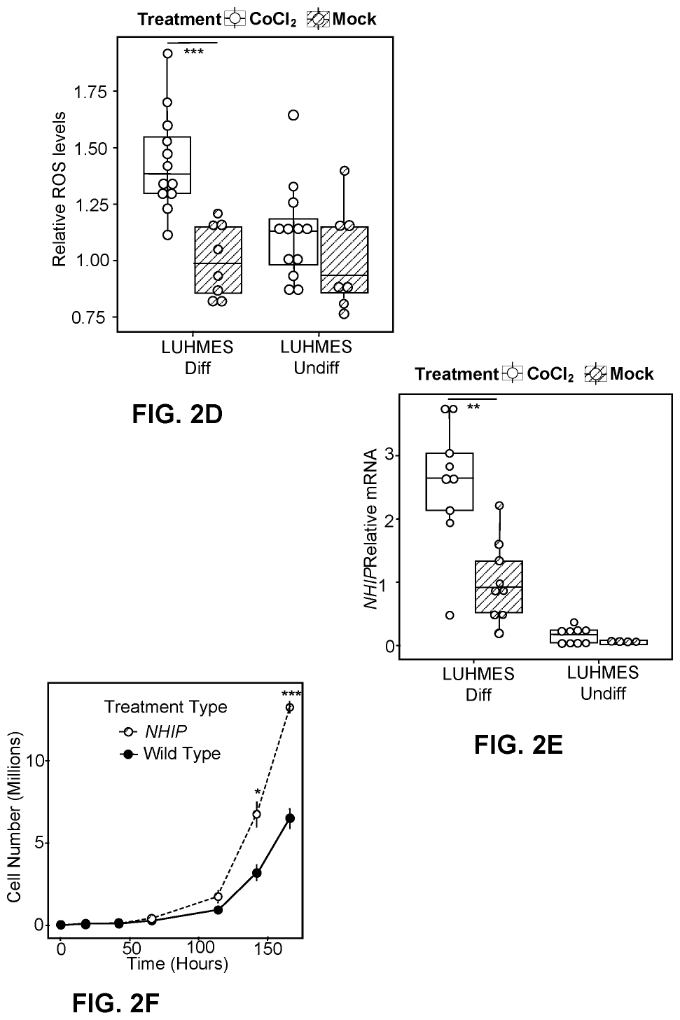

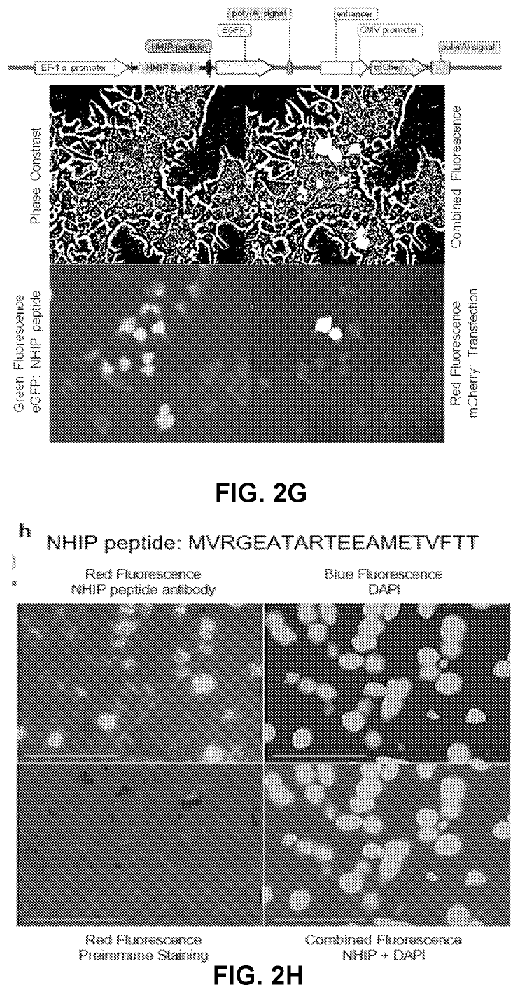

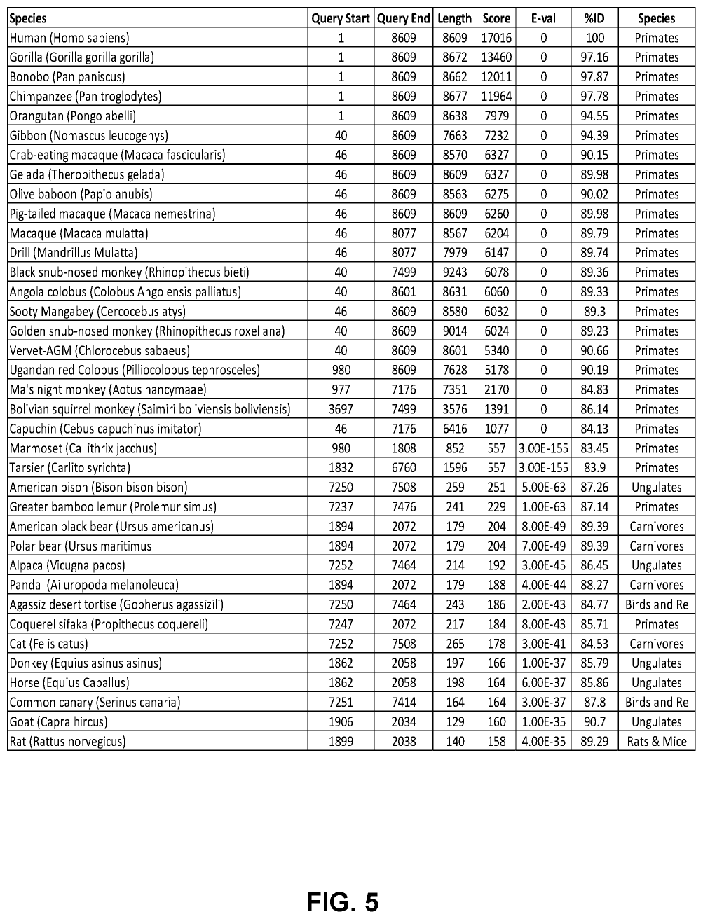

The 22q13.33 co-methylated block was within an apparent gene desert, located more than 500 kb away from the closest annotated protein coding genes: FAM19A5 (TAFA5) and BRD1. Epigenetic evidence for promoter and enhancer activity within 22q13.33 was obtained from placenta, ovary, and brain ENCODE datasets 33. Within 22q13.33, an active promoter peak identified by H3K4me3 histone markers was observed in a subset of ovary, placenta, and brain samples, suggesting variable promoter marks between individuals. This H3K4me3 peak overlapped a CpG island and the TSS of the uncharacterized transcript, LOC105373085 (also named AK057312) identified from a human testis cDNA library³⁴. We renamed LOC105373085 as NHIP, for neuronal hypoxia inducible, placenta associated. NHIP is also variably expressed among brain regions from the Genotype-Tissue Expression (GTEx) database³⁵. The full length NHIP sequence is syntenic in all primates, but not in other vertebrates including mouse (FIG. 5). When quantified by RT-PCR in human tissues, NHIP was expressed in placenta, testis, and adult and fetal brain, with relatively lower expression in placenta (FIG. 2a). ASD placental samples showed significantly lower NHIP transcript levels than TD samples, in the same direction as methylation changes in the 22q13.33 block (FIG. 2b). Since gene body methylation in placenta predicts active gene expression and the 22q13.33 co-methylated block mapped to a previously-defined partially methylated domain in placenta 15, these results suggest that hypomethylation of the 22q13.33 block in ASD is reflective of lower past or current expression of NHIP expression in utero for ASD compared to TD.

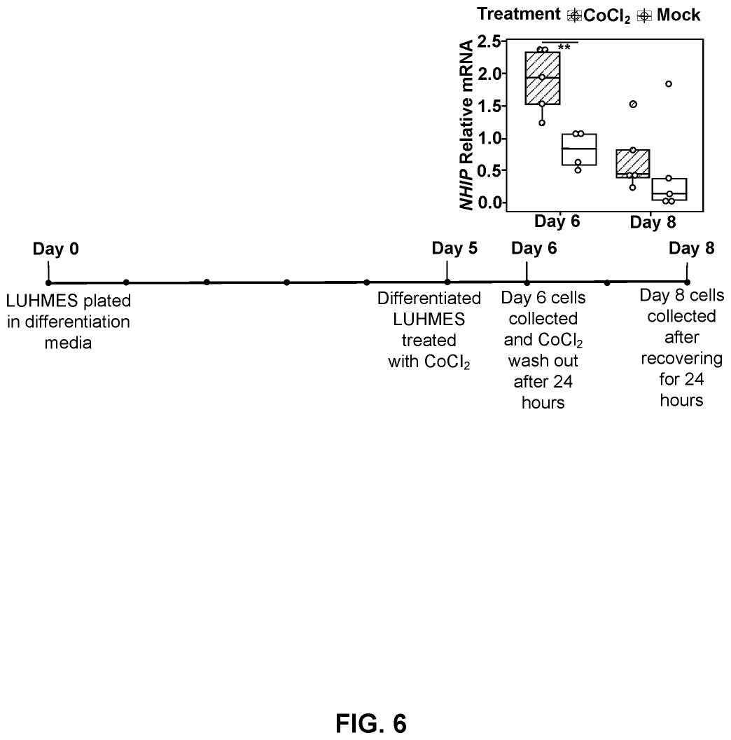

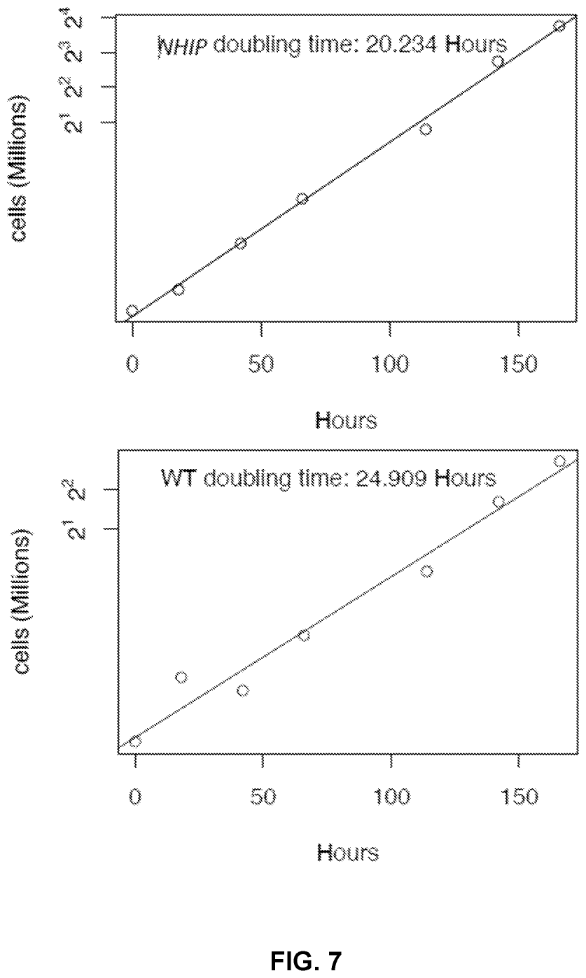

To understand the function of this uncharacterized gene, we assayed and detected levels of NHIP expression in multiple human cell lines (IMR90, LUHMES, SH-SY5Y). A significant increase in NHIP transcript levels was observed following neuronal differentiation in LUHMES cells (FIG. 2c). Since both neuronal differentiation and placental trophoblast differentiation respond to hypoxic conditions^{36,37}, we tested the responsiveness of NHIP to hypoxia. Differentiated LUHMES neurons were more sensitive to treatment with a hypoxia mimetic (CoCl₂) than undifferentiated, with a significant decrease in cell viability and an increase in reactive oxygen species (ROS) levels (FIG. 2d). NHIP transcript levels also increased after exposure to CoCl₂ specifically in differentiated, but not undifferentiated LUHMES cells (FIG. 2e). Following removal of hypoxia, NHIP transcript levels returned to untreated levels, demonstrating the transience of the response (FIG. 6). Among the tested human cell lines, embryonic origin HEK293T cells had the lowest endogenous NHIP transcript levels (FIG. 2c). Since response to hypoxia is a developmental signal regulating cell proliferation in embryos³⁸, we experimentally tested this hypothesis by transiently transfecting HEK293T cells with either a plasmid encoding NHIP with a dual GFP-Puromycin selection cassette or a control vector control lacking NHIP. A significantly shortened doubling time was observed in response to NHIP overexpression compared to control cells (20.23 hour vs. 24.91 hour) (FIG. 2f, FIG. 7). These results demonstrate that NHIP is a hypoxia inducible gene in neurons that regulates cell proliferation in an embryonic cell line with low endogenous expression.

To examine whether NHIP encoded a protein, we identified a 20 amino acid (aa) putative peptide containing a Kozak sequence and tested the existence of the peptide by designing the NHIP-peptide-eGFP vector so that the peptide sequence would be in frame with ATG-less GFP transfected in HEK293T cells (FIG. 2g). The presence of both transfection control (red, mCherry) and reporter (green, eGFP) confirmed the existence of the 20 aa NHIP peptide (FIG. 2g). The NHIP encoded peptide sequence was confirmed using mass spectrometry after pull-down with anti-GFP antibody. A blat search of human databases demonstrated that the NHIP peptide partially overlapped protein sequences within BRCA2 and CHD4. Lastly, using a custom antibody against the NHIP encoded peptide, immunostaining was performed on sections of human postmortem prefrontal cortex, demonstrating nuclear staining in a subset of neuronal nuclei (FIG. 2h). Together, these results demonstrate the existence of a nuclear peptide encoded by NHIP.

A Common Genetic Structural Variant at 22q13.33 is Associated with Reduced Placental DNA Methylation, Reduced NHIP Expression, and Increased ASD Risk

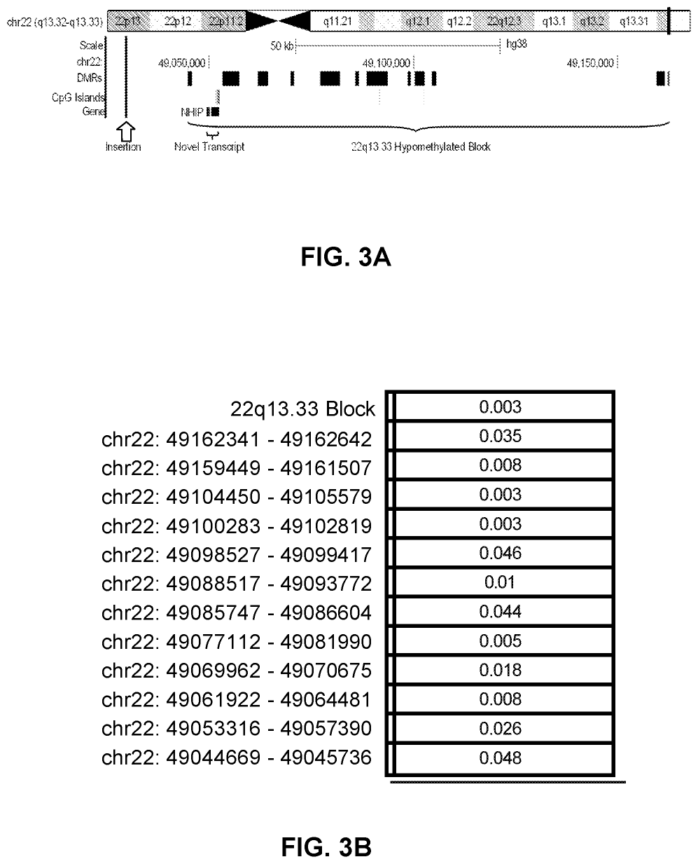

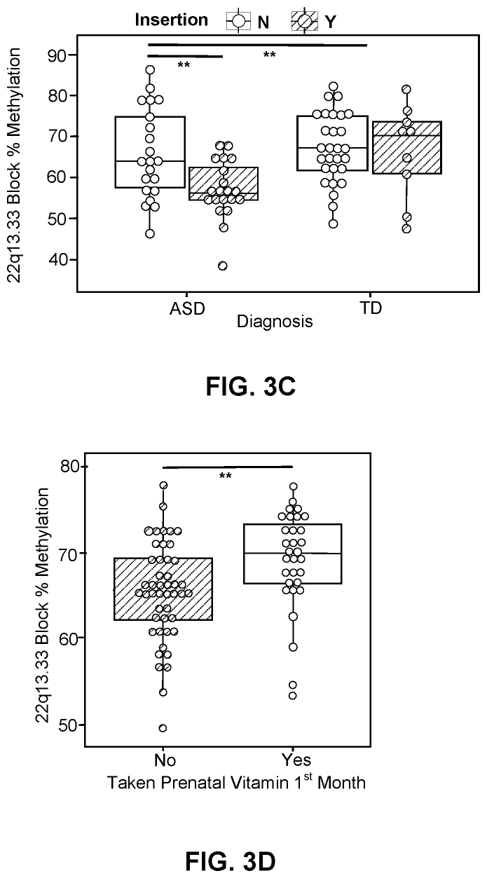

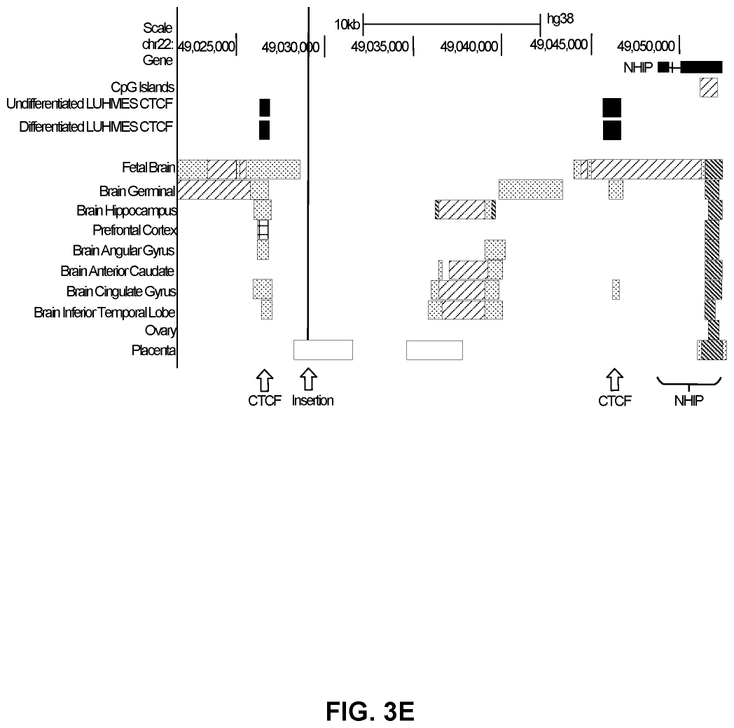

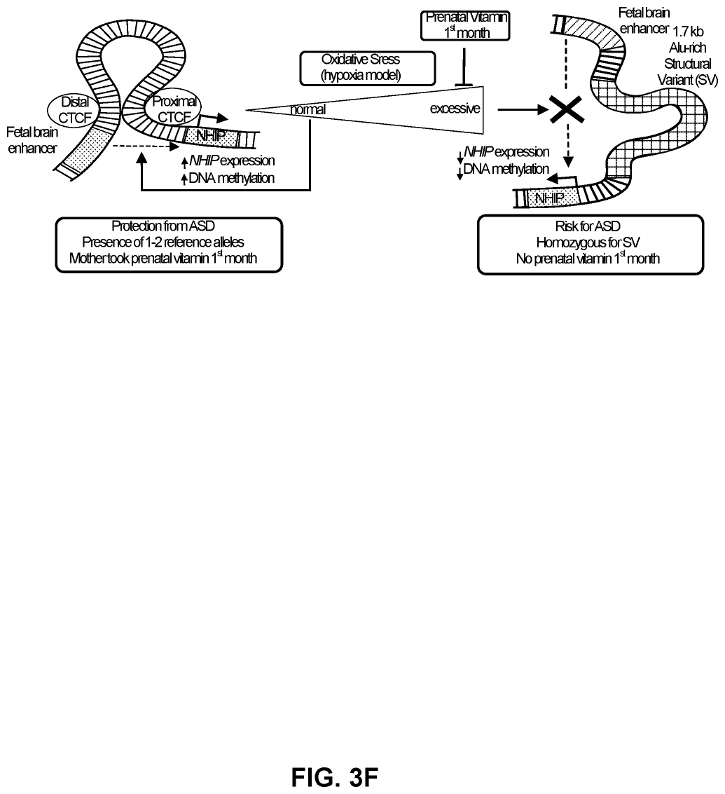

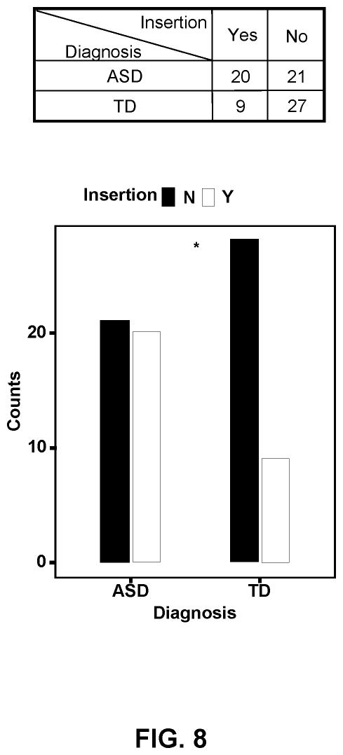

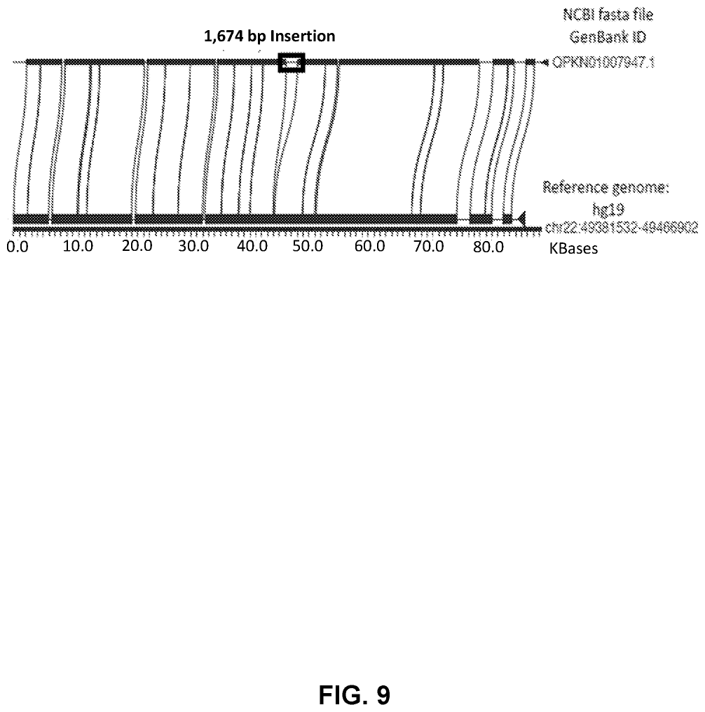

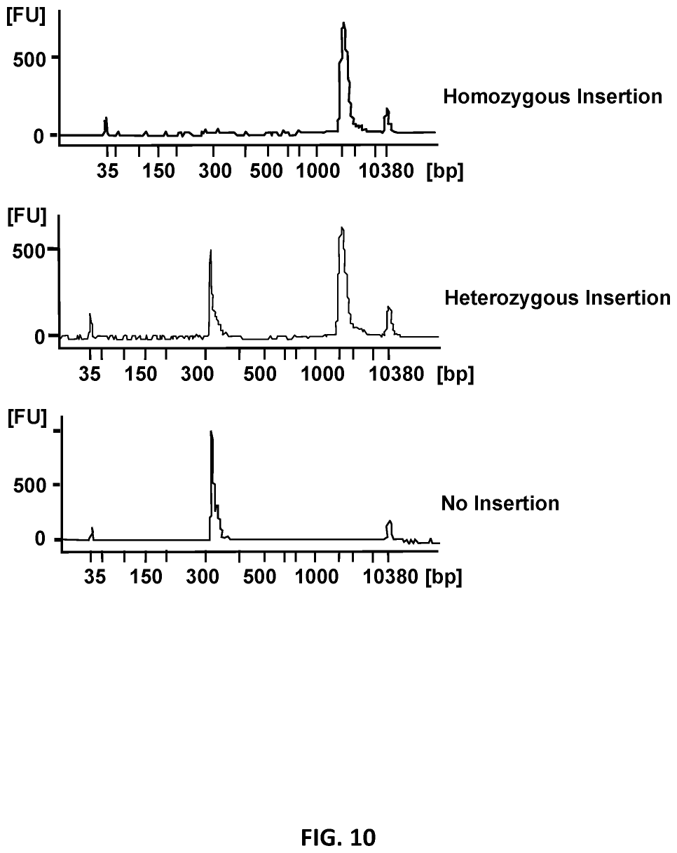

To examine genetic factors associated with 22q13.33 methylation levels and polymorphic expression of NHIP in ASD, we tested the association between 22q13.33 block DNA methylation levels and common variants from individual-matched whole-genome sequencing (WGS), including SNPs, insertions or deletions (indels), copy number variations (CNVs), and SVs. Methylation levels in five out of 12 ASD DMRs within 22q13.33 were significantly associated with common SNPs located inside the DMRs. An upstream 1,674 bp SV insertion (chr22: 49029657, hg38) located 15,013 bp from the start site of the 22q13.33 co-methylated block (FIG. 3a) was identified with which DNA methylation levels of all 12 22q13.33 ASD DMRs were significantly associated (FIG. 3b). In the MARBLES cohort, this SV insertion was identified in significantly more ASD than TD samples (FIG. 8). Placenta samples with the 22q13.33 insertion from ASD, but not TD, showed significantly lower methylation levels (FIG. 3c). While not present in the reference genome, the 22q13.33 insertion was also identified as structural variant identified from PacBio assembly data of the human CHM1 complete hydatidiform cell line (CHM1_chr22-49029645-INS-1673 contig³⁹ and NCBI GenBank ID QPKN01007947.1⁴⁰ (FIG. 9). We also confirmed the WGS identification of SV using PCR genotyping primer sets that confirmed the results (FIG. 10). The insertion sequence showed high similarity with retrotransposon elements, including SVA and Alu. This 22q13.33 SV also corresponded to INS_22_115103 in Genome Aggregation Database (gnomAD)+^{41,42}.

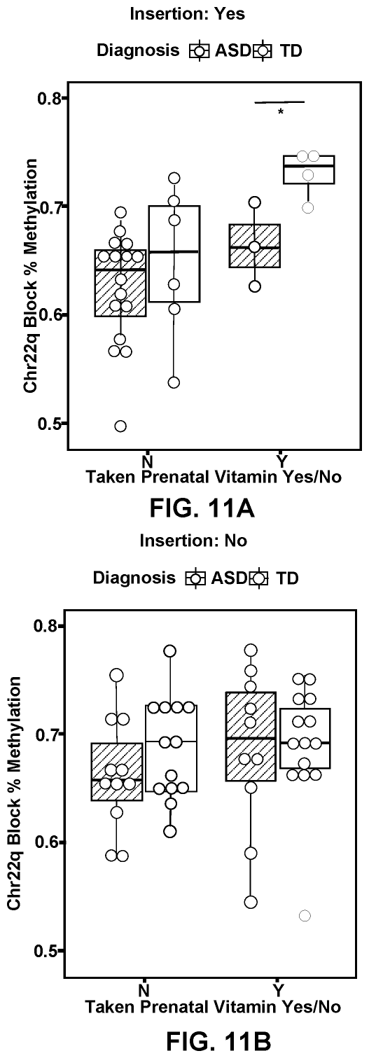

Since the 22q13.33 block exhibited lower methylation in ASD compared to TD placental samples, we chose to evaluate the relationship between prenatal vitamin use during the first month of pregnancy, previously shown to be associated with decreased ASD risk 43, in the context of ASD risk associated with the insertion. There was a significant positive association with prenatal vitamins use in the first month and methylation level at the 22q13.33 block, in the protective direction (FIG. 3d). When samples were stratified by 22q13.33 insertion genotype, prenatal vitamin use during the first month of pregnancy showed a significant protective effect, specifically in individuals with the insertion (FIG. 11). Unlike the 22q13.33 insertion, the GWAS-based PRS⁸ calculated for the MARBLES cohort was not significantly different between diagnostic groups or 22q13.33 block methylation by ANOVA in the MARBLES discovery cohort. Together, these results are consistent with the hypothesis that ASD risk associated with the 22q13.33 SV and co-methylated block is distinguishable from polygenic ASD risk and tempered by a common nutrient intervention with demonstrated ASD protection.

Since SVs have been previously implicated in altering chromatin loops regulating promoter-enhancer interactions⁴⁴, we hypothesized that this 1.7 kb insertion may be located within an enhancer-promoter loop relevant to fetal brain. Using the recent EpiMap database of chromatin states across multiple humans and tissue types⁴⁵, we identified two CTCF sites flanking the SV insertion (FIG. 3e). ChromHMM maps³¹ demonstrate a fetal brain enhancer that aligns with the distal CTCF binding site. The proximal CTCF site is adjacent to the NHIP TSS, which ChromHMM predicts as an active promoter in brain, ovary, and placenta. These two CTCF binding sites were inside a large ˜2 Mb topologically associated domain (TAD) spanning from the 48.5 Mb position to the telomere of 22q⁴⁶. Together, these results suggest a model whereby the SV insertion allele disrupts the fetal brain enhancer-promoter interaction within a large telomeric TAD, thereby reducing the responsiveness of NHIP expression to neuronal differentiation and excessive oxidative stress (FIG. 3f). Early pregnancy prenatal vitamin use is expected to counteract the effects of oxidative stress through provision of dietary methyl groups, thereby increasing DNA methylation at the NHIP locus in individuals homozygous for the 22q13.33 insertion.

NHIP Expression is Reduced in ASD Brain and Associated with the Regulation of Genes Enriched for Synaptic Functions and ASD Risk

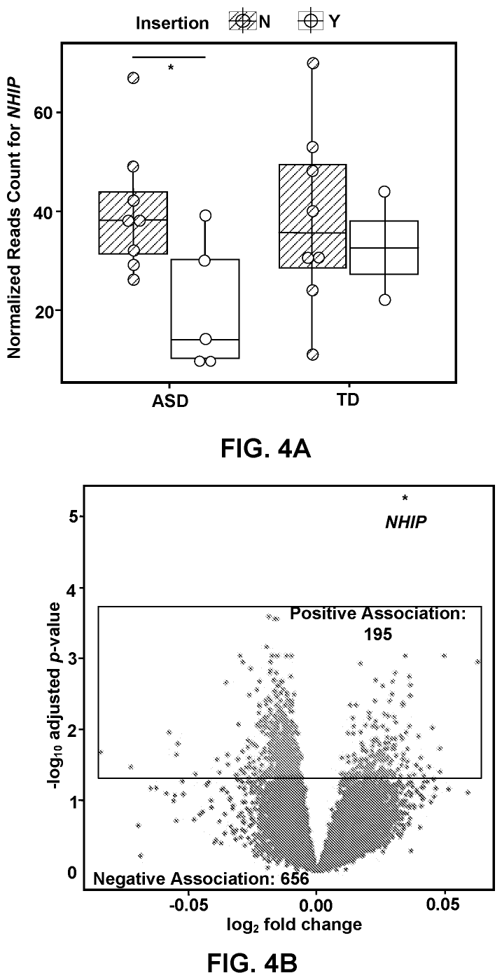

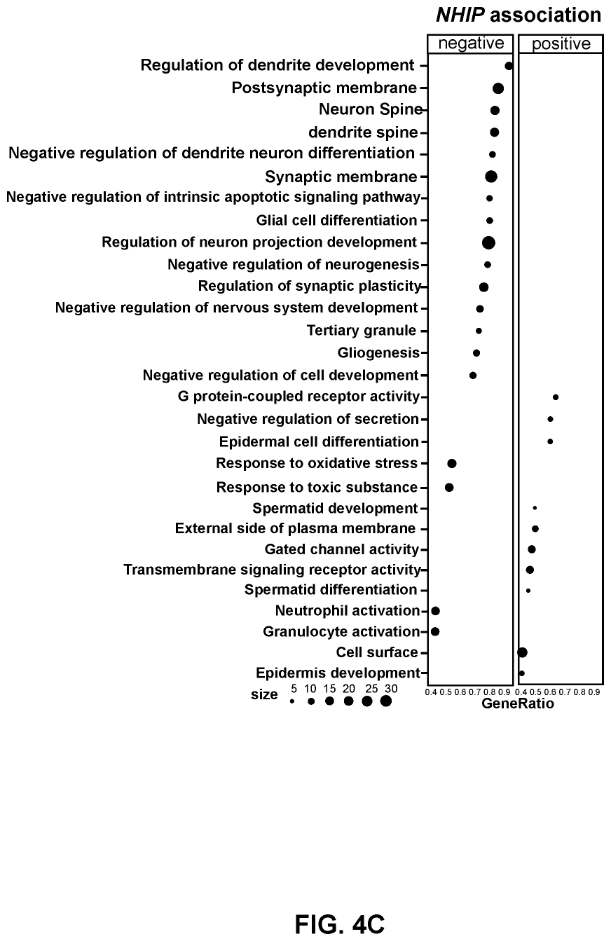

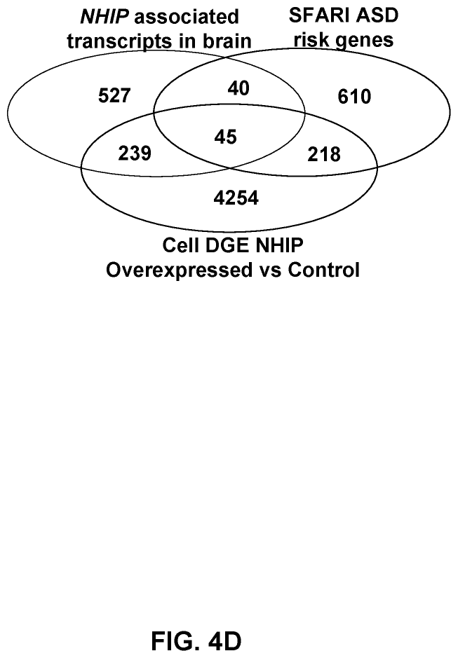

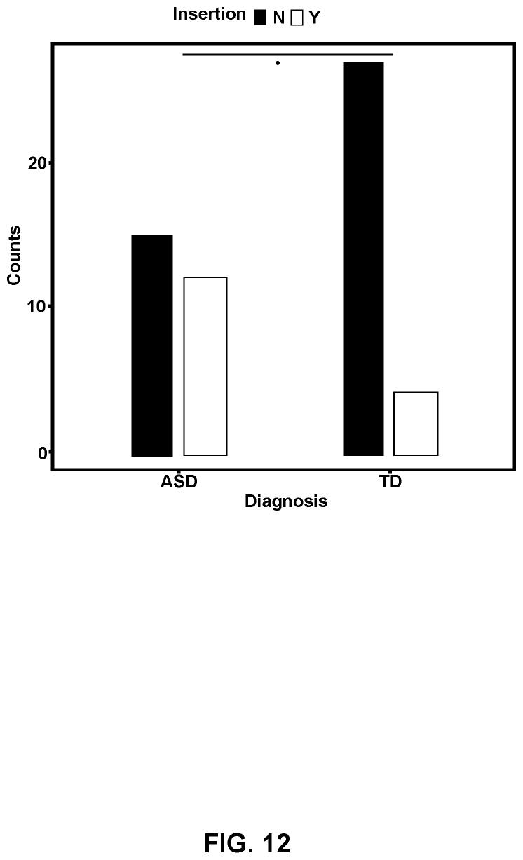

We then tested the hypothesis that the 22q13.33 insertion was associated with NHIP expression in ASD versus TD postmortem brain. Similar to the MARBLES cohort of placenta samples, the 22q13.33 insertion showed a significantly higher frequency in ASD compared with TD in 58 cortical samples (FIG. 12). RNA-seq was performed on a subset of 20 cortical samples representing all three SV insertion genotypes, matched for age and sex between ASD and TD. Brain samples homozygous for the 22q13.33 insertion (Y) exhibited lower NHIP levels compared to those with one or no insertion alleles (N) specifically in ASD, but not in TD samples (FIG. 4a).

We then performed a genome-wide analysis of transcript levels associated with variable NHIP transcript levels in brain samples as a continuous trait. 851 NHIP-associated genes passed FDR significance, including 195 positively and 656 negatively associated (FIG. 4b). Downregulated genes included ASD candidate genes such as (HD) 8⁴⁷, and a gene previously implicated in ASD from placenta, IRS2¹⁷. Gene ontology (GO) enrichment analysis of NHIP-associated genes revealed 277 significant terms (FIG. 4c). Regulation of nervous system, glial cell differentiation, synaptic membrane, neurogenesis, and response to oxidative stress were negatively associated with NHIP transcript levels (FIG. 4c). GO term functions related to the dendritic spine, synaptic plasticity, and regulation of synaptic transmission formed a functional module of genes negatively associated with NHIP levels. In contrast, transcripts positively associated with NHIP levels were enriched for distinct functions in epidermal development, G-protein coupled receptors, and negative regulation of secretion. To further examine the relevance of NHIP expression to ASD etiology, we overlapped brain NHIP-associated transcripts with SFARI ASD risk genes and observed a significant overlap of 85 genes. The 85 genes in common were significantly enriched for 49 GO terms, including nervous system development, synapse, and dendrite, demonstrating associations of NHIP levels with functionally relevant gene pathways in brain and ASD.

### Overexpression of NHIP in HEK293T Cells Results in Large-Scale Transcriptional Changes to Genes Relevant to Brain and ASD Risk

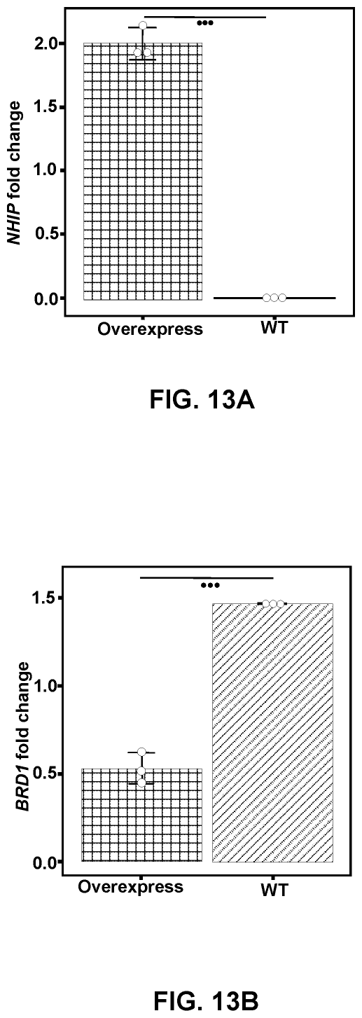

To experimentally model the transcriptional impact of NHIP induction, RNA-seq and differential expression analyses were performed on HEK293T cells transiently transfected with NHIP or vector control. We identified 4,756 differentially expressed genes (DEG) with genome-wide significance (FDR adjusted p-value<0.05). NHIP overexpression increased expression of 1,490 genes and decreased expression of 3,266 genes. Genes decreased with NHIP expression included the downstream flanking gene BRD1, as well as IRS2, CHD8, and DLL1. NHIP overexpression and reduced BRD1 in overexpression cell lines were confirmed with RT-PCR (FIG. 13). Genes differentially expressed with NHIP overexpression were enriched for GO terms associated with non-coding RNA processing, histone modification, placental development, cell cycle, and p53 binding, consistent with the proliferation phenotype (FIG. 2f). KEGG gene set enrichment analysis⁴⁸ showed enrichment for brain disorders, including Parkinson's, Alzheimer's, and Huntington's diseases and metabolism, such as fatty acid metabolism and drug metabolism, further demonstrating the relevance of NHIP regulated genes to brain functions.

In a comparison of in vivo and in vitro RNA-seq analyses, a significant overlap of 284 genes was observed between those differentially expressed in response to experimental NHIP overexpression and those associated with NHIP transcript levels in human brain. Genes negatively associated with NHIP levels in vitro and in vivo were enriched for functions in synapse, dendrite, cell-cell signaling, regulation of nervous system development, and cell cycle. Furthermore, genes differentially expressed with NHIP overexpression also showed a significant overlap of 263 genes with ASD risk genes from the SFARI database enriched for functions in central nervous system development, synaptic signaling, and response to oxygen levels. There were 45 genes in common among ASD risk, NHIP association in brain, and NHIP overexpression, including BRD4, SETD5, CHD2, EP300, and FOXG1 (FIG. 4d, Table 1, Table 2). Genes common to ASD risk, NHIP association in brain, and NHIP overexpression were enriched for chromatin organization, regulation of transcription by RNA polymerase II, regulation of cell differentiation, neurogenesis, rhythmic processes, and response to decreased oxygen (Table 1). Together, these results demonstrate that NHIP is a novel regulatory gene with functions relevant to known ASD risk factors.

### Discussion

This study has taken the innovative approach of utilizing placental tissue from a high-risk prospective pregnancy cohort with multi-omic assays to discover a novel ASD risk gene locus that integrates responsiveness to oxidative stress with inheritance of a common structural variant. Given the distinctive DNA methylation landscape of the placenta characterized by partially methylated domains and higher gene body methylation over expressed genes¹³⁻¹⁵, using unbiased WGBS as a tool enabled the identification of a novel gene associated with ASD that had been missed by standard genetic and epigenetic array-based approaches. The 22q13.33 co-methylated block identified in this study was previously identified by WGBS as a region of increased methylation variance (CORSIV)^{20,21} as well as a region of increased SV 26 in the human genome. We confirmed the hypothesis that CORSIV and SV locations overlap more frequently than expected at random. Although this 22q13.33 region has not been previously associated with ASD risk, the neighboring distal long arm of 22q13.3 harbors multiple genes implicated in neuropsychiatric disorders, including ASD, intellectual disability, schizophrenia, and bipolar disease⁴⁹⁻⁵¹. SHANK3, which encodes a postsynaptic protein required for maturation of glutamatergic synapses⁵², is 1.5 Mb telomeric from the 22q13.33 hypomethylated block identified in this study. Rare SHANK3 mutations are noted in ASD⁵³, and large structural variations including SHANK3 are observed in rare ASD children⁴⁹. In addition, 22q13.33, 22q13.32, and 22q13.31 are disease-associated hotspot regions in ASD²⁹. While these highly polymorphic regions of the genome have the potential to contain regulatory genes such as NHIP, as well as primate-specific sequences relevant to brain development⁵⁴, they are often excluded from the design of array-based platforms because of their complexities. The NHIP locus is sparsely covered by probes in the most current genetic and epigenetic array designs, a likely explanation for why it was not identified by prior ASD studies. In contrast, sequencing-based approaches, such as the integrated WGS and WGBS approach employed here, are a promising alternative for disease association testing.

Placenta is an often misunderstood and overlooked tissue, despite its importance in regulating and thereby reflecting events critical to brain development in utero. Placenta regulates metabolism and provides steroid hormones as well as neurotransmitters critical for the developing brain^{55,56}. Additionally, placenta regulates oxygen supply, as it consumes 40-60% of the body's oxygen, and hypoxia metabolic adaptation regulates trophoblast cell fate decisions^{57,58}. Oxygen tension can also modulate extravillous trophoblast proliferation, differentiation, and invasion⁵⁹, all important for successful implantation and placentation, which can all impact brain development and ASD risk⁶⁰⁻⁶².

We have demonstrated that NHIP is a primate-specific, variably expressed gene responsive to hypoxia in human placenta and brain tissues. The variability in NHIP transcript levels was influenced by both non-genetic and genetic factors. First, NHIP was induced with neuronal differentiation, but also with hypoxia and oxidative stress. Interestingly, the responsiveness of NHIP expression as well as oxidative stress was specific to differentiated neurons but not seen in the undifferentiated state. Oxidative stress is a common convergent mechanism that occurs in normal neurodevelopment but can be excessive in cases of many environmental exposures associated with in ASD, including air pollution⁶³ and pesticides⁶⁴. Second, prenatal vitamin use in the first month of pregnancy provides essential methyl donors to the one-carbon metabolism pathway^{65,66} that may counteract excessive oxidative stress, a prediction consistent with the elevated methylation over the 22q13.33 block in placentas from pregnancies with first month prenatal vitamin use. Third, common genetic variants were also associated with 22q13.33 methylation levels. While we identified 12 SNPs within the 22q13.33 co-methylated block that were significantly associated with methylation, the strongest genetic factor was a 1.7 kb insertion with a high allele frequency in all ethnicities. Homozygosity for this 22q13.33 insertion was a better predictor of ASD risk than GWAS-based PRS. 22q13.33 SV homozygosity was also strongly associated with hypomethylation of this locus and reduced expression of NHIP in ASD compared to TD placenta and brain samples.

Large insertions such as the 22q13.33 SV that occur outside of coding regions can still modify gene expression through alterations in promoter-enhancer loop size. The NHIP promoter shows differences in active chromatin marks between individuals and is associated with two CTCF binding sites that apparently anchor an intra-TAD loop between the promoter and a distal fetal brain enhancer. These results suggest a model by which the presence of at least one copy of the reference allele without the insertion would allow NHIP to be induced during neurodevelopment and hypoxia, thereby protect the developing brain through its regulation of downstream regulatory gene pathways (FIG. 3f). Homozygosity for the 22q13.33 SV allele is associated with lower NHIP expression and less protection, likely because the enhancer-promoter loop forms less efficiently because of the >15% increased size of the loop. For the minority of TD children who were also homozygous for the 22q13.33 SV, the use of prenatal vitamins that reduce the consequences of oxidative stress might have been one source of protection from risk, although other genetic and environmental factors not investigated may also be involved.

### Example 2. Materials and Methods

### Sample Population and Diagnostic Classification

The Markers of Autism Risk in Babies-Learning Early Signs (MARBLES) study⁶⁷ recruited mothers with at least one child that had been diagnosed with ASD and who were pregnant or planning another pregnancy in Northern California, primarily through lists provided by the California Department of Development Services^{17,67-69}. The following criteria were required for MARBLES study's enrollment: the prospective child has at least one first or second degree relative diagnosed with ASD; the mother is at least 18 years old; the mother is pregnant or planning for a pregnancy; the mother speaks, reads and understands English proficiently enough in order to complete the protocol; the mother lives within 2.5 h drive distance of Davis/Sacramento region. Demographic, diet and medical information were collected by prospectively telephone interviews or questionnaires throughout the pregnancy. For this analysis, a discovery set of 46 placentae from children subsequently diagnosed with ASD and 46 placentae from children subsequently found to have typical neurodevelopment (TD) was sequenced. An internal WGBS replication group included 65 additional MARBLES placenta samples (ASD n=21, Non-TD n=13, TD n=31). Finally, whole genome sequence data were available on 41 ASD and 37 TD MARBLES children, which were used for SNP and SV analyses to characterize WGBS findings.

The Early Autism Risk Longitudinal Investigation (EARLI) study recruited pregnant mothers who already have a child diagnosed with ASD and has been described in detail previously⁷⁰. EARLI families were recruited from four sites (Drexel/Children's Hospital of Philadelphia, Johns Hopkins/Kennedy Krieger Institute, Kaiser Permanente Northern California, and University of California, Davis) across three US regions (Southeast Pennsylvania, Northeast Maryland, and Northern California). Enrollment criteria for EARLI were: having a biological child diagnosed with ASD; communicating fluently in English or Spanish; being 18 years or older; living within 2 hour drive distance from the study site; being less than 29 weeks of pregnancy. For replication analysis of the initial MARBLES WGBS findings, 47 placenta samples (ASD n=16, TD n=31) were available from the EARLI study, with details described previously⁶⁴.

In both MARBLES and EARLI studies, the subsequent child diagnosis was clinically assessed by trained, professional examiners at 36 months using standardized instruments including the Autism Diagnostic Observation Schedule (ADOS)⁷¹, Autism Diagnostic Interview-Revised (ADI-R)⁷², and Mullen Scales of Early Learning (MSEL)⁷³. Based on a previously published algorithm, children were classified into three outcome groups: ASD, TD and Non-TD^{43,74,75}. Children with ASD had scores over the ADOS cutoff and fit ASD DSM-5 criteria. Children with TD had all MSEL scores within 2 standard deviations (SD) and no more than one MSEL subscale 1.5 SD below the normative mean together, with scores on the ADOS at least three points lower than the ASD cutoff. Children with Non-TD did not meet ASD or TD criteria, but had elevated ADOS scores and low MSEL scores, defined as two or more MSEL subscales with more than 1.5 SD below normative mean or at least one MSEL subscale more than 2 SD below normative mean.

### Whole Genome Bisulfite Sequencing (WGBS) Library Preparation

The placental samples were frozen within 4 hours after birth. DNA was extracted from placenta tissue with the Gentra Puregene kit (Qiagen, Hilden, Germany) and quantified with the Qubit DNA Assay Kit (Thermo Fisher Scientific, Waltham, MA, USA). The discovery group included 92 samples (ASD n=46, TD n=46) from the MARBLES study. DNA was bisulfite converted with the EZ DNA Methylation Lightning kit (Zymo, Irvine, CA, USA). WGBS libraries were prepared from bisulfite-converted DNA using the TruSeq DNA Methylation kit (Illumina, San Diego, CA, USA) with indexed PCR primers and a 14 cycle PCR programs. Libraries were sequenced at 2 per lane with 150 bp paired-end reads in Illumina HiSeq X (San Diego, CA, USA) by Novogene (Sacramento, CA, USA). The external replication group included 47 samples (ASD n=16, TD n=31) from the EARLI study, with details described previously⁷⁶. The internal replication group included 65 samples (ASD n=21, Non-TD n=13, TD n=31) from the MARBLES study. DNA were sonicated to ˜ 350 bp using Covaris E220 (Woburn, MA, USA). Sonicated and size selected DNA was bisulfite converted using the EZ DNA Methylation Lightning kit (Zymo, Irvine, CA, USA). WGBS libraries were prepared using Accel-NGS Methyl-Seq DNA library kit (Swift Biosciences, Ann Arbor, MI, USA) with indexed PCR primers and a 12 cycle PCR programs. Libraries were pooled and sequenced on 2 lanes with 150 bp paired-end reads of Illumina NovaSeq 6000 S4 (San Diego, CA, USA) by DNA Tech Core at University of California, Davis (Davis, CA, USA).

### WGBS Alignment and Quality Control

Raw sequencing files were preprocessed, aligned to the human reference genome and converted to CpG methylation count matrices with the default parameters in CpG_Me⁷⁷⁻⁷⁹. Reads were trimmed to remove adapters and methylation bias on both 5′ and 3′ end. After trimming, reads were aligned to human reference genome hg38, and filtered for PCR duplicates. Cytosine methylation reports were generated using all covered sites CpG methylation. Quality control was examined for each sample. Libraries with CHH methylation greater than 2% were excluded as incomplete bisulfite conversion. The CpG_Me workflow incorporates Trim Galore, Bismark, Bowtie2, SAMtools, and MultiQC^{78,80-83}.

### Window Methylation and Principal Component Analysis (PCA)

DNA methylation at 20 kb windows sliding across the genome was extracted using getMeth function in bsseq^{84,85} Percent methylation for each sample at each window was calculated using the average methylation value from the window. Correlations between DMRs were calculated using Pearson's correlation coefficient (r). Principal components analysis (PCA) was performed using the prcomp function in the stats package and visualized using ggbiplot⁸⁶. The ellipses for each group were illustrated as the 95% confidence.

### Methylation Array Analysis and Cell Type Estimation

The same 92 placenta DNA samples aliquots in the discovery group (ASD n=46, TD n=46) were used for DNA methylation array analysis. DNA was treated and cleaned with the EZ DNA methylation gold kit (Zymo, Irvine, CA, USA). Samples were assayed on the Infinium MethylationEPIC array (Illumina, San Diego, CA, USA) at John Hopkins University CIDR (Baltimore, MD, USA). Raw image files were analyzed using minfi package⁸⁷. Data were corrected for background and dye bias with the normal-exponential by out-of-band probe (noob) method⁸⁸. Cell type composition of placenta (trophoblast cells, stromal cells, Hofbauer cells, endothelial cells, and nucleated red blood cells) were estimated from methylation using a sorted placenta cell reference using PlaNET⁸⁹.

### Detection of DMRs

DMRs were identified between ASD and TD in the discovery group through DMRichR, with 100 permutations and adjustments for sex and cell types^{77,90}. DMRichR utilized the dmrseq and bsseq algorithms to process methylation levels from CpG count matrix to identify DMRs^{84,91}. The DMR analysis approach used a smoothing and weighting algorithm that weights CpGs based on coverage. CpGs in physical proximity with similar methylation values were grouped into candidate background regions to estimate region statistics. Permutation testing was done on the pooled null distribution to calculate empirical p-values to identify significant DMRs and then further corrected for genome-wide significance at an FDR of 0.05. Individual smoothed methylation levels and chr22q block methylation levels were obtained using bsseq⁸⁴. Genes were assigned to DMRs using the Genomic Regions Enrichment of Annotation Tool (GREAT) tool with the default association settings (5 kb upstream, 1 kb downstream and 1000 kb max extension)⁹². The distances (kb) were calculated from DMRs to the transcription start sit (TSS) of the GREAT assigned genes. Gene Ontology (GO) enrichment analysis for DMRs, hypermethylation DMRs, and hypomethylation DMRs relative to background regions was done using GREAT⁹². Significant terms were called with FDR corrected p-values less than 0.05.

### Placenta DMR Enrichment Analysis

DMRs were examined for enrichment with chromatin marks compared to the background regions using LOLA R package with Fisher's exact test after FDR correction⁹³. Chromatin states were predicted by chromHMM using the Hidden Markov Model to separate human genome into 15 functional states in the Roadmap Epigenomics Project^{31,94}. Promoter related states included active TSS (TssA) (red), TSS flank (TssAFInk) (orange red), bivalent TSS (TssBiv) (Indian Red), and bivalent TSS flank (BivFInk) (Dark Salmon) states. Enhancer related states included genic enhancer (EnhG) (Green Yellow), enhancer (Enh) (Yellow), and bivalent enhancer (EnhBiv) (Dark Khaki). CpG island, shore, shelf and open sea coordinates were obtained from the annotatr R package⁹⁵. Encyclopedia of DNA Elements (ENCODE) datasets were used to extract histone post-translational modifications (PTMs), including H3K4me1, H3K4me3, H4K9me3, H3K36me3, H3K27me3 and H3K27ac datasets^{33,96}. Enrichment for known transcription factor binding site motif sequences in DMRs was obtained using Hypergeometric Optimization of Motif EnRichment (HOMER)⁹⁷.

### Participant Whole Genome Sequencing (WGS) and Variant Calling

WGS was performed using cord blood genomics data on subset of the same individuals from in the discovery group (ASD n=41, TD n=37). Sequencing libraries were generated using NEBNest DNA library prep kit (NEB, Ipswich, MA, USA) with 150 bp paired-end reads in Illumina HiSeq X (San Diego, CA, USA) by Novogene (Sacramento, CA, USA) with at least 30× coverage per sample. Raw read files were mapped to human reference genome hg38 using Burrows-Wheeler Aligner (BWA) with the default setting⁹⁸. SAMtools was utilized to sort the bam files and Picard was used to merge bam files from the same sample identify duplicate reads^{82,99}. Single nucleotide polymorphisms (SNPs), small insertion, and deletions (InDels) were called using GATK and annotated variant using ANNOVAR^{100,101}. Copy number variations (CNVs), longer than 50 bp, were identified using control-FREEC and CREST^{102,103}. Structural variants (SVs) detection and genotyping, larger than 50 bp were performed using DELLY with the default settings¹⁰⁴.

### Polygenic Risk Scores (PRS) Generation

A subset of individuals from in the discovery group were also genotyped using Illumina Multi-Ethnic genotyping array (ASD n=31, TD n=35). Stringent QC criteria was used on the raw genotypes in order to remove low quality SNPs and samples¹⁰⁵. Our criteria included removal of samples with call rates<98%, sex discrepancy, and relatedness (pi-hat<0.18) to non-familial samples with filtering for minor allele frequency (MAF)<5% using PLINK software¹⁰⁶. After data cleaning, the imputation pipeline was performed using University of Michigan Imputation Server¹⁰⁷ using minimac4 software¹⁰⁸ to the 1000G Phase v5 reference panel (hg19)^{109,110}. Phasing was performed using Eagle software¹¹¹.

PRS calculation was performed on the imputed genetic data, after applying post-imputation filtering (R-squared>0.80). PRS was informed by discovery GWAS results from the combined PGC-iPSYCH genome-wide meta-analysis⁸ and generated at a range of p_{discovery} thresholds (p_{discovery} threshold range from 1*10⁻⁸ to 1.0). Using PLINK software¹⁰⁶ we removed correlated SNPs and applied from 2 to >20,000 effect sizes to achieve a weighted summation of alleles, representing a PRS for ASD risk. After evaluating via logistic regression the R² from a model of ASD on ASD-PRS ranging across the discovery thresholds and adjusting for genetic ancestry, we determined that a p_{discovery} of 0.05 achieved the best fit, and thus used this score in further analyses. The association of 22q13.33 co-methylated block % methylation and diagnosis with PRS was measured by analysis of variance (ANOVA), with PRS as the dependent variable.

### Participant Genomic Insertion Characterization and Sanger Sequencing

To validate the 22q13.33 insertion from Illumina WGS data, the expected genomic location of the insertion was queried in a published PacBio long read sequencing dataset³⁹. The insertion was identified located at CHM1_chr22-49029645-INS-1673 contig³⁹. The contig was in a fasta file with accession number GCA 003709635.1 with the correspondence table, it also named with GenBank ID QPKN01007947.1 in NCBI database⁴⁰. SAMtools was utilized to isolate the fasta sequence from the contig with 85,271 bp in length and extracted the insertion sequence with 1,673 bp in length. The QPKN01007947.1 contig mapped to chr22: 49,381,532-49,466,902 (reference genome: hg19) using blat¹¹² and visualized the insertion using Miropeats¹¹³.

In addition to characterizing the insertion using PacBio long read sequencing, primer sets were designed to span the insertion location for PCR-based genotyping. A 25 ul PCR reaction mixture contained 100 ng genomics DNA, 5 μl 5× LongAmp Taq reaction buffer (NEB, Ipswich, MA, USA), 1 μl LongAmp Taq DNA polymerase (NEB, Ipswich, MA, USA), 1 μl 10 mM dNTPs and 2 μl of 10 μM forward and reverse primer. The PCR amplifications were performed using following conditions: initial denaturation at 94° C. for 30 s; 30 cycles of denaturing at 94° C. for 30 s, 52° C. for 30 s and 65° C. for 2 min with a final extension at 65° C. for 10 min. PCR products were subjected to Topoisomerase (TOPO) PCR Cloning Kit (Thermo Fisher Scientific, Waltham, MA, USA) followed by a 1.5% agarose gel electrophoresis with purification and Sanger sequencing by University of California, Davis, DNA Sequencing Facility (Davis, CA, USA) and chromatograms were analyzed using SnapGene (Genewiz, South Plainfield, NJ, USA). PCR products genotype and size were characterized using Bioanalyzer 2100 (Agilent, Santa Clara, CA, USA). The sequence of the insertion was analyzed for repetitive elements using CENSOR and RepeatMasker^{114,115}.

### Cell Culture, Cell-based Assays, and Transfection

LUHMES cells (ATCC, Manassas, VA, USA, CRL-2927) were seeded on fibronectin coated plates (Thermo Fisher Scientific, Waltham, MA, USA, CWP001, 354402). Undifferentiated cells were maintained in proliferation medium: Advanced DMEM/F12 (Invitrogen, Carlsbad, CA, USA), supplemented with N₂ supplement (Invitrogen, Carlsbad, CA, USA), Penicillin-streptomycin-glutamine (Thermo Fisher Scientific, Waltham, MA, USA), and 40 ng/ml recombinant bFGF (Invitrogen, Carlsbad, CA, USA). To generate differentiated LUHMES, cells were switched to differentiation media for five days. Differentiation media: Advanced DMEM/F12, supplemented with N₂ supplement, Penicillin-streptomycin-glutamine, 1 mM dbcAMP (MilliporeSigma, Burlington, MA, USA), 1 μg/ml tetracycline (Neta Scientific, Hainesport, NJ, USA), and 2 ng/ml recombinant human GDNF (Thermo Fisher Scientific, Waltham, MA, USA). For cell viability and hydrogen peroxide production experiments, differentiated cells were growth in 96-well plates for six days prior to treatment with CellTiter Blue or ROS-Glo visualization reagent (Promega, Madison, WI, USA). Undifferentiated cells were plated in 96-well plates at same densities as differentiated neurons and treated identically for cell viability and hydrogen peroxide measurements. For RNA quantification, cells were maintained in 6-well plates. Challenges with hydrogen peroxide (MilliporeSigma, Burlington, MA, USA), cobalt chloride (Thermo Fisher Scientific, Waltham, MA, USA) or mock treatment were carried out after five days of differentiation and cells were treated for 24 hours before analysis.

An overexpression NHIP plasmid, NHIP-eGFP was synthesized by VectorBuilder (Chicago, IL, USA) with EF-1α as promoter for NHIP and CMV as promoter for eGFP fused with puromycin resistant gene. A control plasmid was cut using XbaI and AbaI restriction endonucleases based on the NHIP-eGFP, named NEG-eGFP to remove NHIP and maintained the rest of plasmid structure. Plasmid for NHIP plasmid, NHIP-peptide-eGFP was synthesized by VectorBuilder with EF-1α as promoter for the NHIP peptide, removed the stop codon and fused the end of the NHIP peptide with eGFP, together with CMV as promoter for mCherry fused with puromycin resistant gene (FIG. 2g). All constructs were sequenced by Sanger sequencing by University of California, Davis, DNA Sequencing Facility (Davis, CA, USA) and analyzed using SnapGene (Genewiz, South Plainfield, NJ, USA) to confirm the expected sequence.

HEK293T cells (ATCC, Manassas, VA, USA, CRL-11268) were grown in DMEM/F12, GlutaMAX medium (Thermo Fisher Scientific, Waltham, MA, USA) supplemented with MEM non-essential amino acids (Thermo Fisher Scientific, Waltham, MA, USA) and 10% fetal bovine serum (Invitrogen, Carlsbad, CA, USA) together with Penicillin-streptomycin-glutamine. Low passage HEK293T cells were transfected with plasmids using Lipofectamine 3000 and Opti-MEM (Invitrogen, Carlsbad, CA, USA) according to the manufacturer's instructions. Transfections were performed using HEK293T cell lines for each condition. Transfection medium was replaced 24 h post-transfection with complete growth media with puromycin at 3 μg/ml for 7 days.

All cells were maintained at 37° C. containing 95% O₂ and 5% CO₂. Images were taken using EVOS under magnification labeled in the images. Cell numbers were measured using disposable countess chamber slide on Countess II FL automated cell counter (Thermo Fisher Scientific, Waltham, MA, USA) under the default steps with mixing 10 μl of samples with 10 μl of trypan blue. CellTiter Blue reagent was used for measured cell viability using luminescence based on manufacturer instruction (Promega, Madison, WI, USA). H₂O₂ production represented relative reactive oxygen species (ROS) level was measured with the ROS-Glo H₂O₂ assay system using 50 nM with the default setting with level measured by luminometer (Promega, Madison, WI, USA).

HEK293T whole cell lysates were prepared by resuspension in 1×RIPA buffer and sonication using a Diagenode Bioruptor 300 (Diagenode, Denville, NJ, USA) followed by centrifugation at 21,130×g at 4° C. to remove insoluble material and then resolved on a 4-15% SDS-PAGE gel (Biorad, Hercules, CA, USA). The SDS-PAGE gel was rinsed in three changes of water to remove SDS and stained with Imperial protein stain (Thermo Fisher Scientific, Waltham, MA, USA) to visualize proteins. Stained bands between 25 kd and 37 kd were carefully excised from the gel, washed in three changes of 50 mM ammonium bicarbonate followed by three washes with acetonitrile then swollen in 10 mM DTT in acetonitrile and incubated at 56° C. for 30 minutes do reduce disulfide bonds. The gel pieces were next shrunk by incubation in acetonitrile then incubated in 55 mM iodoacetamide (IAA) in 50 mM ammonium bicarbonate prior to washing with 50 mM ammonium bicarbonate, shrunk with acetonitrile and dried in a speed vac. Gel pieces were suspended in 50 mM ammonium bicarbonate with 0.01% Protease Max (Promega, Madison, WI, USA) and treated with trypsin (Promega) for four hours at 50° C. The NHIP/GFP fusion protein was detected from the resulting peptides by (LC/MS-MS). MS was performed at University of California, Davis Proteomics Core Facility.

NHIP peptide immunofluorescence staining utilized a custom polyclonal antibody that was produced in Rabbit by GenScript Inc (Piscataway, NJ, USA) to a truncated NHIP peptide MVRGEATARTEEAMC (SEQ ID NO:3) and affinity purified. Flash frozen human cortical tissues were fixed in 4% formaldehyde in 1×PBS for 72 hours then dehydrated by immersion in 70% ethanol for seven days and embedded in paraffin. 5 μm sections were cut from embedded brain tissue and mounted on glass slides then baked for 4 hours at 56° C. Tissues on slides were washed four changes of xylene to remove paraffin. Next, slides were washed in two changes of 100% ethanol which was removed by heating to 50° C. on a heat block. The slides were then treated with 1×DAKO antigen retrieval solution (Agilent, Santa Clara, CA, USA) at 95° C. for one hour in a water bath. Slides were washed five times in 1× PBS with agitation. To reduce endogenous autofluorescence slides were immersed in 1×PBS and exposed to LED light for 24 hours. Slides were next incubated with 1×PBS/0.5% Tween 20/3% BSA 1 hour at 37° C. to block background signals then washed three times in 1× PBS/0.5% Tween 20. Anti-NHIP peptide and control pre-immune antibodies were diluted 1/200 in 1×PBS/0.5% Tween 20/3% BSA and incubated on slides at 37° C. overnight in a humid chamber before three washes in 1×PBS/0.5% Tween. Goat anti-Rabbit Alexa 594 (Thermo Fisher Scientific, Waltham, MA, USA, Catalog #A32740) was diluted in 1×PBS/0.5% Tween20/3% BSA with 5 μg/ml DAPI and added to slides for two hours at 37° C. in a humid chamber. Slides were washed five times in 1×PBS/0.5% Tween 20 with shaking before mounting in 5 μg/ml DAPI in 50% glycerol and application of glass coverslips.

RNA Extraction, cDNA Synthesis and RT-PCR

Total RNA was isolated from HEK293T cells transiently transfected with NHIP-eGFP or negative control NEG-eGFP using AllPrep DNA/RNA/Protein mini kit (Qiagen, Hilden, Germany). Human tissue total RNA samples were obtained commercially, including placenta (Life Technology, Carlsbad, CA, USA), testes (TaKaRa Bio, Kusatsu, Shiga, Japan), and fetal brain (Cell Applications, San Diego, CA, USA). RNA was extracted from frozen placenta samples in the Discovery group samples using TRIzol Reagent (Invitrogen, Carlsbad, CA, USA). cDNA was synthesized using the High-Capacity cDNA Reverse Transcription Kit (Thermo Fisher Scientific, Waltham, MA, USA) based on the manufacturer's protocol. TaqMan Gene Expression Assays for 1.0 (105373085 (renamed as NHIP) (assay ID: Hs01034248_s1), BRD1 (Hs00205849_m1), FAM19A5 (Hs00395354_m1) and GAPDH (assay ID: Hs02786624_g1) were used (Thermo Fisher Scientific, Waltham, MA, USA). The expression of 3 genes of interest and 1 reference genes were examined by real-time TaqMan PCR assay (Thermo Fisher Scientific, Waltham, MA, USA). Expression levels were determined by the probes with optimized primer and probe concentrations. Quantification was accomplished with RT-PCR machine using TaqMan Fast Advanced Master Mix with the default parameters by the manufacturer (Thermo Fisher Scientific, Waltham, MA, USA). Reactions were performed with three biological replicates. Fold changes of transcript levels were measured using the Fluidigm Real-Time PCR Analysis software calculated fold change of gene expression as the delta delta CT normalized to GAPDH (Fluidigm, San Francisco, CA, USA).

### Brain Sample Acquisition

Human brain samples were obtained from the NICHD Brain and Tissue Bank for Developmental Disorders at the University of Maryland (Baltimore, MD, USA). RNA from the frozen human brain was purified using AllPrep DNA/RNA/Protein mini kit (Qiagen, Hilden, Germany).

### RNA-Seq Library Preparation and Sequencing

RNA from cells and brain was prepared for RNA-seq library using Kapa RNA HyperPrep kits (Roche, Basel, Switzerland) together with the QIAseq FastSelect Human ribodepletion kit (Qiagen, Hilden, Germany). Libraries were assessed for quality and quantify on Agilent Bioanalyzer 2100 and pooled for multiplex sequencing with at least 25 million reads with 150 bp paired-end on Illumina NovaSeq 6000 S4 (San Diego, CA, USA) by DNA Tech Core at University of California, Davis (Davis, CA, USA).

### RNA-Seq Data Processing and Differential Gene Expression (DGE)

Raw fastq files were processed and aligned using STAR¹¹⁶. After quality control steps by FASTQC, the count matrixes were generated by featureCounts^{117,118} Count matrixes were filtered for at least one count in any sample. Size factors estimation and normalization were performed by DESeq2¹¹⁹. DGE was generated compared between overexpressed NHIP and negative control cells using DESeq2 (FDR corrected p-value<0.05)¹¹⁹. DGE for brain was analyzed by using normalized read count for NHIP levels as continuous trait using DESeq2 (FDR corrected p-value<0.05)¹¹⁹. Gene overlaps between different experiments were tested for significance using Fisher's exact test in the GeneOverlap R package¹²⁰.

Gene Ontology terms for DGE were identified using clusterProfiler on Gene Set Enrichment Analysis using gseGO function with 1,000 permutation tests¹²¹. Normalized enrichment scores (NES) were calculated for enrichment after correcting for FDR multiple testing. The dotplots illustrate significant GO terms based on GeneRatio, calculated from the number of overlapped genes divided by the total number of genes in the gene set¹²¹. GO terms to be included in the plots were selected based of GeneRatio ranking. The enrichment map was plotted using emapplot function on clustering mutually overlapping gene sets to form functional modules¹²¹. The ridgeplot was plotted using ridgeplot R function to visualize expression distributions of core enriched genes¹²¹. The cnetplot depicted the linkages of genes and biological concepts as networks¹²¹.

### Data and Code Availability

Datasets supporting the conclusions are available in the Gene Expression Omnibus repository (GEO)¹²² at accession number (GSE178206)¹²³. Code and scripts for this study are available on GitHub¹²⁴. The gene abbreviation NHIP for “neuronal hypoxia inducible, placental associated” for LOC105373085 was approved by the HUGO Gene Nomenclature Committee.

### REFERENCES

1. Maenner, M. J. et al. Prevalence of autism spectrum disorder among children aged 8 Years-Autism and developmental disabilities monitoring network, 11 Sites, United States, 2016. MMWR Surveill. Summ. 69, 1-12 (2020).
2. Bourgeron, T. From the genetic architecture to synaptic plasticity in autism spectrum disorder. Nat. Rev. Neurosci. 16, 551-563 (2015).
3. Hallmayer, J. et al. Genetic heritability and shared environmental factors among twin pairs with autism. Arch. Gen. Psychiatry (2011). doi: 10.1001/archgenpsychiatry.2011.76
4. Mazina, V. et al. Epigenetics of autism-related impairment: Copy number variation and maternal infection. J. Dev. Behav. Pediatr. (2015). doi: 10.1097/DBP.0000000000000126
5. Gaugler, T. et al. Most genetic risk for autism resides with common variation. Nat. Genet. (2014). doi: 10.1038/ng.3039
6. Iossifov, I. et al. The contribution of de novo coding mutations to autism spectrum disorder. Nature 515, 216-221 (2014).
7. Sanders, S. J. et al. Insights into Autism Spectrum Disorder Genomic Architecture and Biology from 71 Risk Loci. Neuron 87, 1215-1233 (2015).
8 Grove, J. et al. Identification of common genetic risk variants for autism spectrum disorder. Nat. Genet. (2019). doi: 10.1038/s41588-019-0344-8
9. Clarke, T. K. et al. Common polygenic risk for autism spectrum disorder (ASD) is associated with cognitive ability in the general population. Mol. Psychiatry 21, 419-425 (2016).
10. Satterstrom, F. K. et al. Large-Scale Exome Sequencing Study Implicates Both Developmental and Functional Changes in the Neurobiology of Autism. Cell 180, 568-584.e23 (2020).
11. Schizophrenia Working Group of the Psychiatric Genomics Consortium. Biological insights from 108 schizophrenia-associated genetic loci. Nature 511, 421-427 (2014).
12. Ursini, G. et al. Convergence of placenta biology and genetic risk for schizophrenia. Nat. Med. 24, 792-801 (2018).
13. Smallwood, S. A. & Kelsey, G. De novo DNA methylation: A germ cell perspective. Trends Genet. 28, 33-42 (2012).
14. Schroeder, D. I., Lott, P., Korf, I. & LaSalle, J. M. Large-scale methylation domains mark a functional subset of neuronally expressed genes. Genome Res. 21, 1583-91 (2011).
15. Schroeder, D. I. et al. The human placenta methylome. Proc. Natl. Acad. Sci. U.S.A. 110, 6037-6042 (2013).
16. Schroeder, D. I. et al. Placental methylome analysis from a prospective autism study. Mol. Autism 7, 51 (2016).
17. Zhu, Y. et al. Placental DNA methylation levels at CYP2E1 and IRS2 are associated with child outcome in a prospective autism study. Hum. Mol. Genet. 28, 2659-2674 (2019).
18. Santos, H. P. et al. Evidence for the placenta-brain axis: multi-omic kernel aggregation predicts intellectual and social impairment in children born extremely preterm. Mol. Autism 11, 97 (2020).
19. Corley, M. J. et al. Epigenetic Delay in the Neurodevelopmental Trajectory of DNA Methylation States in Autism Spectrum Disorders. Front. Genet. 10, 907 (2019).
20. Gunasekara, C. J. et al. A genomic atlas of systemic interindividual epigenetic variation in humans. Genome Biol. (2019). doi: 10.1186/s13059-019-1708-1
21. Kessler, N. J., Waterland, R. A., Prentice, A. M. & Silver, M. J. Establishment of environmentally sensitive DNA methylation states in the very early human embryo. Sci. Adv. (2018). doi: 10.1126/sciadv.aat2624
22. Hollox, E. J. et al. Psoriasis is associated with increased B-defensin genomic copy number. Nat. Genet. (2008). doi: 10.1038/ng.2007.48
23. Stefansson, H. et al. Large recurrent microdeletions associated with schizophrenia. Nature (2008). doi: 10.1038/nature07229
24. Sekar, A. et al. Schizophrenia risk from complex variation of complement component 4. Nature (2016). doi: 10.1038/nature16549
25. Gokcumen, O. et al. Refinement of primate copy number variation hotspots identifies candidate genomic regions evolving under positive selection. Genome Biol. (2011). doi: 10.1186/gb-2011-12-5-r52
26. Lin, Y. L. & Gokcumen, O. Fine-scale characterization of genomic structural variation in the human genome reveals adaptive and biomedically relevant hotspots. Genome Biol. Evol. (2019). doi: 10.1093/gbe/evz058
27. Girirajan, S. et al. Relative burden of large CNVs on a range of neurodevelopmental phenotypes. PLOS Genet. (2011). doi: 10.1371/journal.pgen.1002334
28. Pinto, D. et al. Functional impact of global rare copy number variation in autism spectrum disorders. Nature (2010). doi: 10.1038/nature09146
29. Girirajan, S. et al. Refinement and discovery of new hotspots of copy-number variation associated with autism spectrum disorder. Am. J. Hum. Genet. (2013). doi: 10.1016/j.ajhg.2012.12.016
30. Turner, T. N. et al. Genome Sequencing of Autism-Affected Families Reveals Disruption of Putative Noncoding Regulatory DNA. Am. J. Hum. Genet. 98, 58-74 (2016).
31. Ernst, J. & Kellis, M. Chromatin-state discovery and genome annotation with ChromHMM. Nat. Protoc. 12, 2478-2492 (2017).
32. Abrahams, B. S. et al. SFARI Gene 2.0: a community-driven knowledgebase for the autism spectrum disorders (ASDs). Mol. Autism 4, 36 (2013).
33. Sloan, C. A. et al. ENCODE data at the ENCODE portal. Nucleic Acids Res. 44, D726-D732 (2016).
34. Ota, T. et al. Complete sequencing and characterization of 21,243 full-length human cDNAs. Nat. Genet. (2004). doi: 10.1038/ng1285
35. Lonsdale, J. et al. The Genotype-Tissue Expression (GTEx) project. Nature Genetics (2013). doi: 10.1038/ng.2653
36. Ortega, J. A., Sirois, C. L., Memi, F., Glidden, N. & Zecevic, N. Oxygen Levels Regulate the Development of Human Cortical Radial Glia Cells. Cereb. Cortex (2017). doi: 10.1093/cercor/bhw194
37. Hayashi, M. et al. Hypoxia up-regulates hypoxia-inducible factor-la expression through RhoA activation in trophoblast cells. J. Clin. Endocrinol. Metab. 90, 1712-1719 (2005).
38. Simon, M. C. & Keith, B. The role of oxygen availability in embryonic development and stem cell function. Nature Reviews Molecular Cell Biology (2008). doi: 10.1038/nrm2354
39. Audano, P. A. et al. Characterizing the Major Structural Variant Alleles of the Human Genome. Cell 176, 663-675.e19 (2019).
40. Homo sapiens isolate CHM1 chromosome 22 22-49000000:0, whole genome sh-Nucleotide-NCBI. Available at: https://www.ncbi.nlm.nih.gov/nuccore/QPKN01007947. (Accessed: 20th March 2021)
41. Collins, R. L. et al. A structural variation reference for medical and population genetics. Nature (2020). doi: 10.1038/s41586-020-2287-8
42. Collins, R. L. et al. INS_22 115103. Available at: https://gnomad.broadinstitute.org/variant/INS_22_115103?dataset=gnomad_sv_r2_1.
43. Schmidt, R. J., Iosif, A.-M., Guerrero Angel, E. & Ozonoff, S. Association of Maternal Prenatal Vitamin Use With Risk for Autism Spectrum Disorder Recurrence in Young Siblings. JAMA Psychiatry 76, 391 (2019).
44. Kaiser, V. B. & Semple, C. A. Chromatin loop anchors are associated with genome instability in cancer and recombination hotspots in the germline. Genome Biol. 19, (2018).
45. Boix, C. A., James, B. T., Park, Y. P., Meuleman, W. & Kellis, M. Regulatory genomic circuitry of human disease loci by integrative epigenomics. Nature 590, 300-307 (2021).
46. Schmitt, A. D. et al. A Compendium of Chromatin Contact Maps Reveals Spatially Active Regions in the Human Genome. Cell Rep. (2016). doi: 10.1016/j.celrep.2016.10.061
47. Bernier, R. et al. Disruptive CHD8 mutations define a subtype of autism early in development. Cell (2014). doi: 10.1016/j.cell.2014.06.017
48. Kanehisa, M. & Goto, S. KEGG: Kyoto Encyclopedia of Genes and Genomes. Nucleic Acids Research (2000). doi: 10.1093/nar/28.1.27
49. Moessner, R. et al. Contribution of SHANK3 mutations to autism spectrum disorder. Am. J. Hum. Genet. (2007). doi: 10.1086/522590
50. Johannessen, M., Haugen, I. B., Bakken, T. L. & Braaten, Ø. A 22q13.33 duplication harbouring the SHANK3 gene: Does it cause neuropsychiatric disorders? BMJ Case Rep. (2019). doi: 10.1136/bcr-2018-228258
51. Han, K. et al. SHANK3 overexpression causes manic-like behaviour with unique pharmacogenetic properties. Nature (2013). doi: 10.1038/nature12630
52. Marshall, C. R. et al. Structural Variation of Chromosomes in Autism Spectrum Disorder. Am. J. Hum. Genet. (2008). doi: 10.1016/j.ajhg.2007.12.009
53. Pfaender, S. et al. Zinc deficiency and low enterocyte zinc transporter expression in human patients with autism related mutations in SHANK3. Sci. Rep. (2017). doi: 10.1038/srep45190
54. Dennis, M. Y. & Eichler, E. E. Human adaptation and evolution by segmental duplication. Current Opinion in Genetics and Development (2016). doi: 10.1016/j.gde.2016.08.001
55. Carter, A. M. Placental oxygen consumption. Part I: In vivo studies-A review. Placenta (2000). doi: 10.1053/plac.1999.0513
56. Rosenfeld, C. S. The placenta-brain-axis. Journal of Neuroscience Research (2021). doi: 10.1002/jnr.24603
57. Zamudio, S. et al. Human placental hypoxia-inducible factor-la expression correlates with clinical outcomes in chronic hypoxia in vivo. Am. J. Pathol. (2007). doi: 10.2353/ajpath.2007.061185
58. Semenza, G. L. Regulation of oxygen homeostasis by hypoxia-Inducible factor 1. Physiology (2009). doi: 10.1152/physiol.00045.2008
59. Genbacev, O., Zhou, Y., Ludlow, J. W. & Fisher, S. J. Regulation of human placental development by oxygen tension. Science (80-.). (1997). doi: 10.1126/science.277.5332.1669
60. Sun, L. et al. Reduced fetal cerebral oxygen consumption is associated with smaller brain size in fetuses with congenital heart disease. Circulation (2015). doi: 10.1161/CIRCULATIONAHA.114.013051
61. Turner, J. M., Mitchell, M. D. & Kumar, S. S. The physiology of intrapartum fetal compromise at term. American Journal of Obstetrics and Gynecology (2020). doi: 10.1016/j.ajog.2019.07.032
62. Fajersztajn, L. & Veras, M. M. Hypoxia: From Placental Development to Fetal Programming. Birth Defects Research (2017). doi: 10.1002/bdr2.1142
63. Raz, R. et al. Autism Spectrum Disorder and Particulate Matter Air Pollution before, during, and after Pregnancy: A Nested Case-Control Analysis within the Nurses' Health Study II Cohort. Environ. Health Perspect. 123, 264-270 (2015).
64. Roberts, E. M. et al. Maternal residence near agricultural pesticide applications and autism spectrum disorders among children in the California Central Valley. Environ. Health Perspect. (2007). doi: 10.1289/ehp.10168
65. Fagiolini, M., Jensen, C. L. & Champagne, F. A. Epigenetic influences on brain development and plasticity. Current Opinion in Neurobiology (2009). doi: 10.1016/j.conb.2009.05.009
66. Schmidt, R. J. et al. Prenatal vitamins, one-carbon metabolism gene variants, and risk for autism. Epidemiology 22, 476-485 (2011).
67. Hertz-Picciotto, I. et al. A Prospective Study of Environmental Exposures and Early Biomarkers in Autism Spectrum Disorder: Design, Protocols, and Preliminary Data from the MARBLES Study. Environ. Health Perspect. 126, 117004 (2018).
68. Mordaunt, C. E. et al. Cord blood DNA methylome in newborns later diagnosed with autism spectrum disorder reflects early dysregulation of neurodevelopmental and X-linked genes. Genome Med. 12, 88 (2020).
69. Zhu, Y. et al. Expression Changes in Epigenetic Gene Pathways Associated With One-Carbon Nutritional Metabolites in Maternal Blood From Pregnancies Resulting in Autism and Non-Typical Neurodevelopment. Autism Res. (2020). doi: 10.1002/aur.2428
70. Newschaffer, C. J. et al. Infant siblings and the investigation of autism risk factors. J. Neurodev. Disord. 4, 7 (2012).
71. Lord, C. et al. Autism Diagnostic Observation Schedule (ADOS). J. Autism Dev. Disord. 30, 205-223 (2000).
72. Rutter, M., LeCouteur, A. & Lord, C. Autism Diagnostic Interview-Revised (ADI-R). Statew. Agric. L. Use Baseline 2015 1, (2015).
73. Mullen, E. Mullen scales of early learning. (1995).
74. Chawarska, K. et al. 18-month predictors of later outcomes in younger siblings of children with autism spectrum disorder: a baby siblings research consortium study. J. Am. Acad. Child Adolesc. Psychiatry 53, 1317-1327.e1 (2014).
75. Ozonoff, S. et al. The broader autism phenotype in infancy: When does it emerge? J. Am. Acad. Child Adolesc. Psychiatry 53, (2014).
76. Ladd-Acosta, C. et al. Placenta DNA methylation at ZNF300 is associated with fetal sex and placental morphology. bioRxiv 2021.03.05.433992 (2021). doi: 10.1101/2021.03.05.433992
77. Laufer, B. I. et al. Low-Pass Whole Genome Bisulfite Sequencing of Neonatal Dried Blood Spots Identifies a Role for RUNX1 in Down Syndrome DNA Methylation Profiles. Hum. Mol. Genet. (2020). doi: 10.1093/hmg/ddaa218
78. Krueger, F. & Andrews, S. R. Bismark: A flexible aligner and methylation caller for Bisulfite-Seq applications. Bioinformatics (2011). doi: 10.1093/bioinformatics/btr167
79. Coulson, R. L. et al. Snord116-dependent diurnal rhythm of DNA methylation in mouse cortex. Nat. Commun. 9, 1616 (2018).
80. Krueger, F. Trim Galore!: A wrapper tool around Cutadapt and FastQC to consistently apply quality and adapter trimming to FastQ files. Babraham Inst. (2015).
81. Langmead, B. & Salzberg, S. L. Fast gapped-read alignment with Bowtie 2. Nat. Methods (2012). doi: 10.1038/nmeth.1923
82. Li, H. et al. The Sequence Alignment/Map format and SAMtools. Bioinformatics 25, 2078-2079 (2009).
83. Ewels, P., Magnusson, M., Lundin, S. & Kaller, M. MultiQC: Summarize analysis results for multiple tools and samples in a single report. Bioinformatics (2016). doi: 10.1093/bioinformatics/btw354
84. Hansen, K. D., Langmead, B. & Irizarry, R. A. BSmooth: from whole genome bisulfite sequencing reads to differentially methylated regions. Genome Biol. (2012). doi: 10.1186/gb-2012-13-10-R83
85. Mordaunt, C. E. et al. Epigenomic signatures in liver and blood of Wilson disease patients include hypermethylation of liver-specific enhancers. Epigenetics Chromatin 12, 10 (2019).
86. Vu, V. Q. ggbiplot: A ggplot2 based biplot. R package version 0.55. Vu, Vincent Q. (2011).
87. Aryee, M. J. et al. Minfi: a flexible and comprehensive Bioconductor package for the analysis of Infinium DNA methylation microarrays. Bioinformatics 30, 1363-1369 (2014).
88. Triche, T. J., Weisenberger, D. J., Van Den Berg, D., Laird, P. W. & Siegmund, K. D. Low-level processing of Illumina Infinium DNA Methylation BeadArrays. Nucleic Acids Res. (2013). doi: 10.1093/nar/gkt090
89. Yuan, V. et al. Accurate ethnicity prediction from placental DNA methylation data. Epigenetics and Chromatin (2019). doi: 10.1186/s13072-019-0296-3
90. Laufer, B. GitHub-ben-laufer/DMRichR: An executable and package for the statistical analysis and visualization of differentially methylated regions (DMRs) from CpG count matrices (Bismark cytosine reports). Available at: https://github.com/ben-laufer/DMRichR.
91. Korthauer, K., Chakraborty, S., Bei, Y. & Irizarry, R. A. Detection and accurate false discovery rate control of differentially methylated regions from whole genome bisulfite sequencing. Biostatistics 20, 367-383 (2019).
92. McLean, C. Y. et al. GREAT improves functional interpretation of cis-regulatory regions. Nat. Biotechnol. 28, 495-501 (2010).
93. Sheffield, N. C. & Bock, C. LOLA: enrichment analysis for genomic region sets and regulatory elements in R and Bioconductor. Bioinformatics 32, 587-589 (2016).
94. Kundaje, A. et al. Integrative analysis of 111 reference human epigenomes. Nature 518, 317-330 (2015).
95. Cavalcante, R. G. & Sartor, M. A. Annotatr: Genomic regions in context. Bioinformatics (2017). doi: 10.1093/bioinformatics/btx183
96. ENCODE Project Consortium, T. E. P. An integrated encyclopedia of DNA elements in the human genome. Nature 489, 57-74 (2012).
97. Heinz, S. et al. Simple Combinations of Lineage-Determining Transcription Factors Prime cis-Regulatory Elements Required for Macrophage and B Cell Identities. Mol. Cell (2010). doi: 10.1016/j.molcel.2010.05.004
98. Li, H. & Durbin, R. Fast and accurate short read alignment with Burrows-Wheeler transform. Bioinformatics (2009). doi: 10.1093/bioinformatics/btp324
99. Broad Institute. Picard Tools-By Broad Institute. Github (2009).
100. McKenna, A. et al. The Genome Analysis Toolkit: a MapReduce framework for analyzing next-generation DNA sequencing data. Genome Res. 20, 1297-303 (2010).
101. Wang, K., Li, M. & Hakonarson, H. ANNOVAR: functional annotation of genetic variants from high-throughput sequencing data. Nucleic Acids Res. 38, e164-e164 (2010).
102. Boeva, V. et al. Control-FREEC: A tool for assessing copy number and allelic content using next-generation sequencing data. Bioinformatics (2012). doi: 10.1093/bioinformatics/btr670
103. Wang, J. et al. CREST maps somatic structural variation in cancer genomes with base-pair resolution. Nat. Methods (2011). doi: 10.1038/nmeth.1628
104. Rausch, T. et al. DELLY: Structural variant discovery by integrated paired-end and split-read analysis. Bioinformatics (2012). doi: 10.1093/bioinformatics/bts378
105. Anderson, C. A. et al. Data quality control in genetic case-control association studies. Nat. Protoc. 5, 1564-1573 (2010).
106. Purcell, S. et al. PLINK: A tool set for whole-genome association and population-based linkage analyses. Am. J. Hum. Genet. 81, 559-575 (2007).
107. Michigan Imputation Server. Available at: https://imputationserver.sph.umich.edu/index.html #!pages/home. (Accessed: 21st March 2021)
108. Das, S. et al. Next-generation genotype imputation service and methods. Nat. Genet. 48, 1284-1287 (2016).
109. Auton, A. et al. A global reference for human genetic variation. Nature 526, 68-74 (2015).
110. Sudmant, P. H. et al. An integrated map of structural variation in 2,504 human genomes. Nature 526, 75-81 (2015).
111. Loh, P. R. et al. Reference-based phasing using the Haplotype Reference Consortium panel. Nat. Genet. 48, 1443-1448 (2016).
112. Kent, W. J. BLAT—The BLAST-like alignment tool. Genome Res. (2002). doi: 10.1101/gr.229202. Article published online before March 2002
113. Parsons, J. D. Miropeats: graphical DNA sequence comparisons. Bioinformatics 11, 615-619 (1995).
114. Jurka, J. Repeats in genomic DNA: Mining and meaning. Curr. Opin. Struct. Biol. (1998). doi: 10.1016/S0959-440X(98)80067-5
115. RepeatMasker Home Page. Available at: http://www.repeatmasker.org/. (Accessed: 6th January 2021)
116. Dobin, A. et al. STAR: Ultrafast universal RNA-seq aligner. Bioinformatics (2013). doi: 10.1093/bioinformatics/bts635
117. Andrews, S., Krueger, F., Seconds-Pichon, A., Biggins, F. & Wingett, S. FastQC. A quality control tool for high throughput sequence data. Babraham Bioinformatics. Babraham Institute (2015).
118. Liao, Y., Smyth, G. K. & Shi, W. FeatureCounts: An efficient general purpose program for assigning sequence reads to genomic features. Bioinformatics (2014). doi: 10.1093/bioinformatics/btt656
119. Love, M. I., Huber, W. & Anders, S. Moderated estimation of fold change and dispersion for RNA-seq data with DESeq2. Genome Biol. (2014). doi: 10.1186/s13059-014-0550-8
120. Li, S. GeneOverlap. GeneOverlap: Test and visualize gene overlaps (2019). doi: 10.18129/B9.bioc.GeneOverlap
121. Yu, G., Wang, L. G., Han, Y. & He, Q. Y. ClusterProfiler: An R package for comparing biological themes among gene clusters. Omi. A J. Integr. Biol. (2012). doi: 10.1089/omi.2011.0118
122. Edgar, R., Domrachev, M. & Lash, A. E. Gene Expression Omnibus: NCBI gene expression and hybridization array data repository. Nucleic Acids Res. (2002). doi: 10.1093/nar/30.1.207
123. GEO Accession viewer: GSE178206. Available at: https://www.ncbi.nlm.nih.gov/geo/query/acc.cgi?acc-GSE178206.
124. Zhu, Y. GitHub-ASD Epigenetics and Genetics Biomarkers. Available at: https://github.com/Yihui-Zhu/Epigenetics_Genetics_ASD_Biomarker.

### Exemplary Embodiments

Exemplary embodiments provided in accordance with the presently disclosed subject matter include, but are not limited to, the claims and the following embodiments:
1. A method for determining a risk of an offspring for developing an autism spectrum disorder (ASD), the method comprising:
detecting in a biological sample obtained from the offspring, mother or potential mother of the offspring expression and/or DNA methylation of a neuronal hypoxia inducible, placental associated (NHIP) gene, wherein decreased expression and/or decreased methylation of the NHIP gene compared to a control sample indicates an increased risk of the offspring for developing an ASD.
2. The method of embodiment 1, wherein the method further comprises obtaining a biological sample from the mother or potential mother.
3. The method of embodiment 1 or 2, wherein the biological sample is selected from the group consisting of blood, serum, plasma, or saliva from the mother, and placenta, cord blood, blood, saliva and brain from the offspring.
4. The method of any one of embodiments 1-3, wherein the mother or potential mother has a child with an ASD.
5. The method of any one of embodiments 1-4, wherein the mother or potential mother has a familial history of ASD.
6. The method of any one of embodiments 1-5, wherein the offspring is a fetus or child.
7. The method of any one of embodiments 1-6, wherein the control sample is selected from a mother or potential mother having an offspring without an ASD or an offspring exhibiting typical development.
8 The method of any one of embodiments 1-7, wherein the detecting step comprises detecting DNA methylation of the NHIP genetic locus, the chr22q13.33 hypomethylated block, or both.
9. The method of embodiment 8, wherein lower DNA methylation levels indicates an increased risk of the offspring for developing an ASD.
10. The method of any one of embodiments 1-7, wherein detecting expression of the NHIP gene comprises detecting an RNA expressed by the NHIP gene or detecting a peptide encoded by the RNA.
11. The method of embodiment 10, wherein the RNA is transcribed from an open reading frame comprising the DNA sequence

(SEQ ID NO: 2)
ATGGTGAGAGGAGAGGCCACCGCACGAACGGAAGAAGCGATGGAGACGG
TCTTTACGACC.

12. The method of embodiment 10 or 11, wherein detecting an RNA expressed by the NHIP gene is selected from amplifying the RNA, quantifying the RNA, or sequencing the RNA.
13. The method of embodiment 10, wherein detecting a peptide encoded by the RNA is selected from i) contacting the peptide with a primary antibody that binds the peptide and detecting the primary antibody with a labeled secondary antibody, ii) linking the peptide to a detectable label, or iii) by immunostaining.
14. The method of embodiment 10 or 13, wherein the peptide comprises the amino acid sequence MVRGEATARTEEAMETVFTT (SEQ ID NO:1).
15. The method of any one of embodiments 1-14, further comprising administering a vitamin to the mother or potential mother if the mother is homozygous for a structural variant inserted about 15 Kbp upstream from the start site of the chr22q13.33 hypomethylated block.
16. The method of embodiment 15, wherein the vitamin is administered during the first month of pregnancy.
17. The method of embodiment 15 or 16, wherein the vitamin comprises a, one or more, or a plurality of dietary methyl group(s).
18. The method of any one of embodiments 1-17, wherein the NHIP gene is hypomethylated.
19. The method of any one of embodiments 1-18, wherein the biological sample is homozygous for a structural variant insertion (chr22: 49029657, hg38) upstream of the 22q13.33 locus.
20. A method for detecting an NHIP peptide in a subject, the method comprising:
obtaining a biological sample from the subject; and
detecting the presence of the NHIP peptide by contacting the biological sample with an anti-NHIP antibody and detecting binding between the NHIP peptide and the antibody.
21. The method of embodiment 20, wherein the subject is a mother or potential mother of an offspring at risk for developing an ASD.
22. A method for preventing an autism spectrum disorder (ASD) in an offspring, the method comprising:
administering a vitamin to the mother of the offspring before and/or during pregnancy, wherein the mother has decreased expression and/or DNA methylation of the NHIP gene in a biological sample compared to a control sample.
23. A method for preventing or reducing a risk of an offspring for developing an autism spectrum disorder (ASD), the method comprising:
i) selecting a mother or potential mother of the offspring, wherein the mother or potential mother is selected based on having decreased expression and/or DNA methylation of the NHIP gene in a biological sample compared to a control sample; and
ii) administering a vitamin to the mother or potential mother before and/or during pregnancy, thereby preventing or reducing the risk that the offspring develops an ASD.
24. The method of any one of embodiments 20-23, wherein the biological sample is selected from the group consisting of blood, serum, plasma, or saliva from the mother, and placenta, cord blood, blood, saliva and brain from the offspring.
25. The method of embodiment 22 or 23, wherein the control sample is selected from a mother or potential mother having an offspring without an ASD or an offspring exhibiting typical development.
26. A method for preventing or reducing a risk of an offspring for developing an autism spectrum disorder (ASD), the method comprising:
administering a therapeutically effective amount of an NHIP gene, an NHIP RNA, or an NHIP peptide, to the mother of the offspring before and/or during pregnancy, thereby preventing or reducing the risk of the offspring for developing an ASD.
27. A plasmid or vector comprising the NHIP gene, or DNA encoding an NHIP RNA or peptide.
28. The plasmid or vector of embodiment 27, further comprising nucleic acid sequences that regulate transcription and/or translation of the NHIP RNA.
29. An in vitro method for increasing cell proliferation, comprising transfecting a cell with the plasmid or vector of embodiment 27 or 28.
30. A method for regulating gene expression, comprising transfecting a cell with the plasmid or vector of embodiment 27 or 28, and detecting differential expression of one or more genes.
31. An isolated peptide comprising an amino acid sequence having at least about 80% sequence identity to SEQ ID NO:1.
32. A fusion protein comprising the peptide of embodiment 31.
33. A kit comprising reagents for detecting expression of an NHIP RNA or NHIP peptide.
34. An array comprising one or more nucleic acid sequences or probes that are capable of hybridizing to an NHIP RNA.
35. An array comprising one or more agents that bind to an NHIP peptide immobilized on a solid support.
36. The array of embodiment 35, wherein the one or more agents comprise an antigen binding protein that specifically binds to the NHIP peptide.
37. A method for sequencing an NHIP gene sequence, comprising amplifying all or part of an NHIP gene from a biological sample obtained from a subject using a set of primers to produce amplified nucleic acid; and sequencing the amplified nucleic acid.
38. The method of embodiment 37, wherein the subject is a mother or potential mother of an offspring at risk for developing an ASD.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

INFORMAL SEQUENCE LISTING

SEQ ID NO: 1: MVRGEATARTEEAMETVFTT
SEQ ID NO: 2:
ATGGTGAGAGGAGAGGCCACCGCACGAACGGAAGAAGCGATGGAGACGGTCTT
TACGACC
SEQ ID NO: 3: MVRGEATARTEEAMC
SEQ ID NO: 4. NHIP-peptide-eGFP:
TTAACCCTAGAAAGATAGTCTGCGTAAAATTGACGCATGCATTCTTGAAATATTG
CTCTCTCTTTCTAAATAGCGCGAATCCGTCGCTGTGCATTTAGGACATCTCAGTCG
CCGCTTGGAGCTCCCGTGAGGCGTGCTTGTCAATGCGGTAAGTGTCACTGATTTT
GAACTATAACGACCGCGTGAGTCAAAATGACGCATGATTATCTTTTACGTGACTT
TTAAGATTTAACTCATACGATAATTATATTGTTATTTCATGTTCTACTTACGTGAT
AACTTATTATATATATATTTTCTTGTTATAGATATCATCAACTTTGTATAGAAAAG
TTGGGCTCCGGTGCCCGTCAGTGGGCAGAGCGCACATCGCCCACAGTCCCCGAG
AAGTTGGGGGGAGGGGTCGGCAATTGAACCGGTGCCTAGAGAAGGTGGCGCGG
GGTAAACTGGGAAAGTGATGTCGTGTACTGGCTCCGCCTTTTTCCCGAGGGTGGG
GGAGAACCGTATATAAGTGCAGTAGTCGCCGTGAACGTTCTTTTTCGCAACGGGT
TTGCCGCCAGAACACAGGTAAGTGCCGTGTGTGGTTCCCGCGGGCCTGGCCTCTT
TACGGGTTATGGCCCTTGCGTGCCTTGAATTACTTCCACCTGGCTGCAGTACGTG
ATTCTTGATCCCGAGCTTCGGGTTGGAAGTGGGTGGGAGAGTTCGAGGCCTTGCG
CTTAAGGAGCCCCTTCGCCTCGTGCTTGAGTTGAGGCCTGGCCTGGGCGCTGGGG
CCGCCGCGTGCGAATCTGGTGGCACCTTCGCGCCTGTCTCGCTGCTTTCGATAAG
TCTCTAGCCATTTAAAATTTTTGATGACCTGCTGCGACGCTTTTTTTCTGGCAAGA
TAGTCTTGTAAATGCGGGCCAAGATCTGCACACTGGTATTTCGGTTTTTGGGGCC
GCGGGCGGCGACGGGGCCCGTGCGTCCCAGCGCACATGTTCGGCGAGGCGGGGC
CTGCGAGCGCGGCCACCGAGAATCGGACGGGGGTAGTCTCAAGCTGGCCGGCCT
GCTCTGGTGCCTGGTCTCGCGCCGCCGTGTATCGCCCCGCCCTGGGCGGCAAGGC
TGGCCCGGTCGGCACCAGTTGCGTGAGCGGAAAGATGGCCGCTTCCCGGCCCTG
CTGCAGGGAGCTCAAAATGGAGGACGCGGCGCTCGGGAGAGCGGGCGGGTGAG
TCACCCACACAAAGGAAAAGGGCCTTTCCGTCCTCAGCCGTCGCTTCATGTGACT
CCACGGAGTACCGGGCGCCGTCCAGGCACCTCGATTAGTTCTCGAGCTTTTGGAG
TACGTCGTCTTTAGGTTGGGGGGAGGGGTTTTATGCGATGGAGTTTCCCCACACT
GAGTGGGTGGAGACTGAAGTTAGGCCAGCTTGGCACTTGATGTAATTCTCCTTGG
AATTTGCCCTTTTTGAGTTTGGATCTTGGTTCATTCTCAAGCCTCAGACAGTGGTT
CAAAGTTTTTTTCTTCCATTTCAGGTGTCGTGACAAGTTTGTACAAAAAAGCAGG
CTCAGCGGAACACACGGTCCCAAAAGGCCCTTCCCAAGATGGCCGGGGCTTCCG
CCTGCCGCCCTCCCTCCCGTGGTGCTGAGCGCAGGCTGGGCGCGGCCACATCACC
CTAAGGGCGTGGGTGTCGGAGTCTTGCAGGCTGGGGTCCCAGGTGAGAGATGCC
AGCAGGGAGCTCTAGATGGGGAACTGGGTGAGCCTGGCTCGGTAAGGGCGTGCT
CAGAACACCCCATCCCCGAACACGGTCTTGTGTGATAAAATGTCACGCAAGAAG
AATCTGAAACCGCGAGAAGAGGAGGACACGGCCAGACTGCACCGAACCCCGGC
ACCTCTGTGGGGAAAAGCAGGTCAGGCTGAGCGGGGCCGGCGGGGACACGCGCT
CCTGGGCTTCTCCAGAGTCTGCCGGGGCCGGGCCCGGGACCGAGGCTGGGACGC
GCTGTGCAGTCCCACCCCTCACACCCCTCGCACGCCTGGAAACACCCTCGGGGTA
ACACAAGCCGGGGTTGAGTTTCTTGAAGAGAAGCTGGCTGCATCCTGGAGCCAG
GGAAAGAGGAGCACAGGGGCCAAGCGGTCCAGGGCCACGGAGGAGCAGGACCC
CTGGGGAAAGGCCCGGGTCTAGGCCGGGGCCAGGGGACCGTGGTGGAGACCTCA
AAAATGGCAGAACACGGAAGCAGGGCGAGAAAAGTAAACGTAGTCCTTGCGGC
AGTTTGAAATACACGCGGGTAAACGCTGGGTGACTCCGCCCGGATGCAGAGTGG
GGGTCTGTGTCTCTCCCCACAGGCTGCAGGGACCGGGCTCTGGGTAACCAGCAG
AAGGTAACAGAACGAGGCTGCTTTTCCTCCAGGCTGTTCTGGTGTCCGCGCGTGG
CTTGTGCGCTGACTCCTGACTTGGAGCGCCGCGTGGCCAGAGAAATCTGGGTGCC
TCCAGGCCACCATGGTGAGAGGAGAGGCCACCGCACGAACGGAAGAAGCGATG
GAGACGGTCTTTACGACCTCTGGTGGCGGAGGCTCGGGCGGAGGTGGGTCGGGT
GGCGGCGGATCAGTGAGCAAGGGCGAGGAGCTGTTCACCGGGGTGGTGCCCATC
CTGGTCGAGCTGGACGGCGACGTAAACGGCCACAAGTTCAGCGTGTCCGGCGAG
GGCGAGGGCGATGCCACCTACGGCAAGCTGACCCTGAAGTTCATCTGCACCACC
GGCAAGCTGCCCGTGCCCTGGCCCACCCTCGTGACCACCCTGACCTACGGCGTGC
AGTGCTTCAGCCGCTACCCCGACCACATGAAGCAGCACGACTTCTTCAAGTCCGC
CATGCCCGAAGGCTACGTCCAGGAGCGCACCATCTTCTTCAAGGACGACGGCAA
CTACAAGACCCGCGCCGAGGTGAAGTTCGAGGGCGACACCCTGGTGAACCGCAT
CGAGCTGAAGGGCATCGACTTCAAGGAGGACGGCAACATCCTGGGGCACAAGCT
GGAGTACAACTACAACAGCCACAACGTCTATATCATGGCCGACAAGCAGAAGAA
CGGCATCAAGGTGAACTTCAAGATCCGCCACAACATCGAGGACGGCAGCGTGCA
GCTCGCCGACCACTACCAGCAGAACACCCCCATCGGCGACGGCCCCGTGCTGCT
GCCCGACAACCACTACCTGAGCACCCAGTCCGCCCTGAGCAAAGACCCCAACGA
GAAGCGCGATCACATGGTCCTGCTGGAGTTCGTGACCGCCGCCGGGATCACTCTC
GGCATGGACGAGCTGTACAAGTAAACCCAGCTTTCTTGTACAAAGTGGTGATCCT
CAGGTGCAGGCTGCCTATCAGAAGGTGGTGGCTGGTGTGGCCAATGCCCTGGCTC
ACAAATACCACTGAGATCTTTTTCCCTCTGCCAAAAATTATGGGGACATCATGAA
GCCCCTTGAGCATCTGACTTCTGGCTAATAAAGGAAATTTATTTTCATTGCAATA
GTGTGTTGGAATTTTTTGTGTCTCTCACTCGGAAGGACATATGGGAGGGCAAATC
ATTTAAAACATCAGAATGAGTATTTGGTTTAGAGTTTGGCAACATATGCCCATAT
GCTGGCTGCCATGAACAAAGGTTGGCTATAAAGAGGTCATCAGTATATGAAACA
GCCCCCTGCTGTCCATTCCTTATTCCATAGAAAAGCCTTGACTTGAGGTTAGATTT
TTTTTATATTTTGTTTTGTGTTATTTTTTTCTTTAACATCCCTAAAATTTTCCTTACA
TGTTTTACTAGCCAGATTTTTCCTCCTCTCCTGACTACTCCCAGTCATAGCTGTCC
CTCTTCTCTTATGGAGATCCCTCGACCTGCAGCCCAAGCTTCGCGTTGACATTGAT
TATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCC
AACGACCCCCGCCCATTGACGTCAATAATGACGTATGTTCCCATAGTAACGCCAA
TAGGGACTTTCCATTGACGTCAATGGGTGGAGTATTTACGGTAAACTGCCCACTT
GGCAGTACATCAAGTGTATCATATGCCAAGTACGCCCCCTATTGACGTCAATGAC
GGTAAATGGCCCGCCTGGCATTATGCCCAGTACATGACCTTATGGGACTTTCCTA
CTTGGCAGTACATCTACGTATTAGTCATCGCTATTACCATGGTGATGCGGTTTTGG
CAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCC
AAAATGTCGTAACAACTCCGCCCCATTGACGCAAATGGGCGGTAGGCGTGTACG
GTGGGAGGTCTATATAAGCAGAGCTCTCTGGCTAACTAGAGAACCCACTGCGCC
ACCATGGTGAGCAAGGGCGAGGAGGATAACATGGCCATCATCAAGGAGTTCATG
CGCTTCAAGGTGCACATGGAGGGCTCCGTGAACGGCCACGAGTTCGAGATCGAG
GGCGAGGGCGAGGGCCGCCCCTACGAGGGCACCCAGACCGCCAAGCTGAAGGT
GACCAAGGGTGGCCCCCTGCCCTTCGCCTGGGACATCCTGTCCCCTCAGTTCATG
TACGGCTCCAAGGCCTACGTGAAGCACCCCGCCGACATCCCCGACTACTTGAAGC
TGTCCTTCCCCGAGGGCTTCAAGTGGGAGCGCGTGATGAACTTCGAGGACGGCG
GCGTGGTGACCGTGACCCAGGACTCCTCCCTGCAGGACGGCGAGTTCATCTACAA
GGTGAAGCTGCGCGGCACCAACTTCCCCTCCGACGGCCCCGTAATGCAGAAGAA
GACCATGGGCTGGGAGGCCTCCTCCGAGCGGATGTACCCCGAGGACGGCGCCCT
GAAGGGCGAGATCAAGCAGAGGCTGAAGCTGAAGGACGGCGGCCACTACGACG
CTGAGGTCAAGACCACCTACAAGGCCAAGAAGCCCGTGCAGCTGCCCGGCGCCT
ACAACGTCAACATCAAGTTGGACATCACCTCCCACAACGAGGACTACACCATCG
TGGAACAGTACGAACGCGCCGAGGGCCGCCACTCCACCGGCGGCATGGACGAGC
TGTACAAGTAACTCGAGTCTAGAGGGCCCGTTTAAACCCGCTGATCAGCCTCGAC
TGTGCCTTCTAGTTGCCAGCCATCTGTTGTTTGCCCCTCCCCCGTGCCTTCCTTGA
CCCTGGAAGGTGCCACTCCCACTGTCCTTTCCTAATAAAATGAGGAAATTGCATC
GCATTGTCTGAGTAGGTGTCATTCTATTCTGGGGGGTGGGGTGGGGCAGGACAGC
AAGGGGGAGGATTGGGAAGACAATAGCAGGCATGCTGGGGATGCGGTGGGCTCT
ATGGCTCGAGTTAATTAACGAGAGCATAATATTGATATGTGCCAAAGTTGTTTCT
GACTGACTAATAAGTATAATTTGTTTCTATTATGTATAGGTTAAGCTAATTACTTA
TTTTATAATACAACATGACTGTTTTTAAAGTACAAAATAAGTTTATTTTTGTAAAA
GAGAGAATGTTTAAAAGTTTTGTTACTTTATAGAAGAAATTTTGAGTTTTTGTTTT
TTTTTAATAAATAAATAAACATAAATAAATTGTTTGTTGAATTTATTATTAGTATG
TAAGTGTAAATATAATAAAACTTAATATCTATTCAAATTAATAAATAAACCTCGA
TATACAGACCGATAAAACACATGCGTCAATTTTACGCATGATTATCTTTAACGTA
CGTCACAATATGATTATCTTTCTAGGGTTAAATAATAGTTTCTAATTTTTTTATTA
TTCAGCCTGCTGTCGTGAATACCGAGCTCCAATTCGCCCTATAGTGAGTCGTATT
ACAATTCACTGGCCGTCGTTTTACAACGTCGTGACTGGGAAAACCCTGGCGTTAC
CCAACTTAATCGCCTTGCAGCACATCCCCCTTTCGCCAGCTGGCGTAATAGCGAA
GAGGCCCGCACCGATCGCCCTTCCCAACAGTTGCGCAGCCTGAATGGCGAATGG
GACGCGCCCTGTAGCGGCGCATTAAGCGCGGCGGGTGTGGTGGTTACGCGCAGC
GTGACCGCTACACTTGCCAGCGCCCTAGCGCCCGCTCCTTTCGCTTTCTTCCCTTC
CTTTCTCGCCACGTTCGCCGGCTTTCCCCGTCAAGCTCTAAATCGGGGGCTCCCTT
TAGGGTTCCGATTTAGTGCTTTACGGCACCTCGACCCCAAAAAACTTGATTAGGG
TGATGGTTCACGTAGTGGGCCATCGCCCTGATAGACGGTTTTTCGCCCTTTGACG
TTGGAGTCCACGTTCTTTAATAGTGGACTCTTGTTCCAAACTGGAACAACACTCA
ACCCTATCTCGGTCTATTCTTTTGATTTATAAGGGATTTTGCCGATTTCGGCCTAT
TGGTTAAAAAATGAGCTGATTTAACAAAAATTTAACGCGAATTTTAACAAAATAT
TAACGCTTACAATTTAGGTGGCACTTTTCGGGGAAATGTGCGCGGAACCCCTATT
TGTTTATTTTTCTAAATACATTCAAATATGTATCCGCTCATGAGACAATAACCCTG
ATAAATGCTTCAATAATATTGAAAAAGGAAGAGTATGAGTATTCAACATTTCCGT
GTCGCCCTTATTCCCTTTTTTGCGGCATTTTGCCTTCCTGTTTTTGCTCACCCAGAA
ACGCTGGTGAAAGTAAAAGATGCTGAAGATCAGTTGGGTGCACGAGTGGGTTAC
ATCGAACTGGATCTCAACAGCGGTAAGATCCTTGAGAGTTTTCGCCCCGAAGAAC
GTTTTCCAATGATGAGCACTTTTAAAGTTCTGCTATGTGGCGCGGTATTATCCCGT
ATTGACGCCGGGCAAGAGCAACTCGGTCGCCGCATACACTATTCTCAGAATGACT
TGGTTGAGTACTCACCAGTCACAGAAAAGCATCTTACGGATGGCATGACAGTAA
GAGAATTATGCAGTGCTGCCATAACCATGAGTGATAACACTGCGGCCAACTTACT
TCTGACAACGATCGGAGGACCGAAGGAGCTAACCGCTTTTTTGCACAACATGGG
GGATCATGTAACTCGCCTTGATCGTTGGGAACCGGAGCTGAATGAAGCCATACC
AAACGACGAGCGTGACACCACGATGCCTGTAGCAATGGCAACAACGTTGCGCAA
ACTATTAACTGGCGAACTACTTACTCTAGCTTCCCGGCAACAATTAATAGACTGG
ATGGAGGCGGATAAAGTTGCAGGACCACTTCTGCGCTCGGCCCTTCCGGCTGGCT
GGTTTATTGCTGATAAATCTGGAGCCGGTGAGCGTGGGTCTCGCGGTATCATTGC
AGCACTGGGGCCAGATGGTAAGCCCTCCCGTATCGTAGTTATCTACACGACGGG
GAGTCAGGCAACTATGGATGAACGAAATAGACAGATCGCTGAGATAGGTGCCTC
ACTGATTAAGCATTGGTAACTGTCAGACCAAGTTTACTCATATATACTTTAGATT
GATTTAAAACTTCATTTTTAATTTAAAAGGATCTAGGTGAAGATCCTTTTTGATAA
TCTCATGACCAAAATCCCTTAACGTGAGTTTTCGTTCCACTGAGCGTCAGACCCC
GTAGAAAAGATCAAAGGATCTTCTTGAGATCCTTTTTTTCTGCGCGTAATCTGCT
GCTTGCAAACAAAAAAACCACCGCTACCAGCGGTGGTTTGTTTGCCGGATCAAG
AGCTACCAACTCTTTTTCCGAAGGTAACTGGCTTCAGCAGAGCGCAGATACCAAA
TACTGTTCTTCTAGTGTAGCCGTAGTTAGGCCACCACTTCAAGAACTCTGTAGCA
CCGCCTACATACCTCGCTCTGCTAATCCTGTTACCAGTGGCTGCTGCCAGTGGCG
ATAAGTCGTGTCTTACCGGGTTGGACTCAAGACGATAGTTACCGGATAAGGCGC
AGCGGTCGGGCTGAACGGGGGGTTCGTGCACACAGCCCAGCTTGGAGCGAACGA
CCTACACCGAACTGAGATACCTACAGCGTGAGCTATGAGAAAGCGCCACGCTTC
CCGAAGGGAGAAAGGCGGACAGGTATCCGGTAAGCGGCAGGGTCGGAACAGGA
GAGCGCACGAGGGAGCTTCCAGGGGGAAACGCCTGGTATCTTTATAGTCCTGTC
GGGTTTCGCCACCTCTGACTTGAGCGTCGATTTTTGTGATGCTCGTCAGGGGGGC
GGAGCCTATGGAAAAACGCCAGCAACGCGGCCTTTTTACGGTTCCTGGCCTTTTG
CTGGCCTTTTGCTCACATGTTCTTTCCTGCGTTATCCCCTGATTCTGTGGATAACC
GTATTACCGCCTTTGAGTGAGCTGATACCGCTCGCCGCAGCCGAACGACCGAGCG
CAGCGAGTCAGTGAGCGAGGAAGCGGAAGAGCGCCCAATACGCAAACCGCCTCT
CCCCGCGCGTTGGCCGATTCATTAATGCAGCTGGCACGACAGGTTTCCCGACTGG
AAAGCGGGCAGTGAGCGCAACGCAATTAATGTGAGTTAGCTCACTCATTAGGCA
CCCCAGGCTTTACACTTTATGCTTCCGGCTCGTATGTTGTGTGGAATTGTGAGCGG
ATAACAATTTCACACAGGAAACAGCTATGACCATGATTACGCCAAGCTCGAAAT
TAACCCTCACTAAAGGGAACAAAAGCTGGTACCTCGCGCGACTTGGTTTGCCATT
CTTTAGCGCGCGTCGCGTCACACAGCTTGGCCACAATGTGGTTTTTGTCAAACGA
AGATTCTATGACGTGTTTAAAGTTTAGGTCGAGTAAAGCGCAAATCTTTT
SEQ ID NO: 5: NEG-eGFP:
GATCTTTTTCCCTCTGCCAAAAATTATGGGGACATCATGAAGCCCCTTGAGCATC
TGACTTCTGGCTAATAAAGGAAATTTATTTTCATTGCAATAGTGTGTTGGAATTTT
TTGTGTCTCTCACTCGGAAGGACATATGGGAGGGCAAATCATTTAAAACATCAGA
ATGAGTATTTGGTTTAGAGTTTGGCAACATATGCCCATATGCTGGCTGCCATGAA
CAAAGGTTGGCTATAAAGAGGTCATCAGTATATGAAACAGCCCCCTGCTGTCCAT
TCCTTATTCCATAGAAAAGCCTTGACTTGAGGTTAGATTTTTTTTATATTTTGTTTT
GTGTTATTTTTTTCTTTAACATCCCTAAAATTTTCCTTACATGTTTTACTAGCCAGA
TTTTTCCTCCTCTCCTGACTACTCCCAGTCATAGCTGTCCCTCTTCTCTTATGGAGA
TCCCTCGACCTGCAGCCCAAGCTTCGCGTTGACATTGATTATTGACTAGTTATTAA
TAGTAATCAATTACGGGGTCATTAGTTCATAGCCCATATATGGAGTTCCGCGTTA
CATAACTTACGGTAAATGGCCCGCCTGGCTGACCGCCCAACGACCCCCGCCCATT
GACGTCAATAATGACGTATGTTCCCATAGTAACGCCAATAGGGACTTTCCATTGA
CGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTAC
GTATTAGTCATCGCTATTACCATGGTGATGCGGTTTTGGCAGTACATCAATGGGC
GTGGATAGCGGTTTGACTCACGGGGATTTCCAAGTCTCCACCCCATTGACGTCAA
TGGGAGTTTGTTTTGGCACCAAAATCAACGGGACTTTCCAAAATGTCGTAACAAC
TCCGCCCCATTGACGCAAATGGGCGGTAGGCGTGTACGGTGGGAGGTCTATATA
AGCAGAGCTCTCTGGCTAACTAGAGAACCCACTGCGCCACCATGGTGAGCAAGG
GCGAGGAGCTGTTCACCGGGGTGGTGCCCATCCTGGTCGAGCTGGACGGCGACG
TAAACGGCCACAAGTTCAGCGTGTCCGGCGAGGGCGAGGGCGATGCCACCTACG
GCAAGCTGACCCTGAAGTTCATCTGCACCACCGGCAAGCTGCCCGTGCCCTGGCC
CACCCTCGTGACCACCCTGACCTACGGCGTGCAGTGCTTCAGCCGCTACCCCGAC
CACATGAAGCAGCACGACTTCTTCAAGTCCGCCATGCCCGAAGGCTACGTCCAG
GAGCGCACCATCTTCTTCAAGGACGACGGCAACTACAAGACCCGCGCCGAGGTG
AAGTTCGAGGGCGACACCCTGGTGAACCGCATCGAGCTGAAGGGCATCGACTTC
AAGGAGGACGGCAACATCCTGGGGCACAAGCTGGAGTACAACTACAACAGCCAC
AACGTCTATATCATGGCCGACAAGCAGAAGAACGGCATCAAGGTGAACTTCAAG
ATCCGCCACAACATCGAGGACGGCAGCGTGCAGCTCGCCGACCACTACCAGCAG
AACACCCCCATCGGCGACGGCCCCGTGCTGCTGCCCGACAACCACTACCTGAGC
ACCCAGTCCGCCCTGAGCAAAGACCCCAACGAGAAGCGCGATCACATGGTCCTG
CTGGAGTTCGTGACCGCCGCCGGGATCACTCTCGGCATGGACGAGCTGTACAAG
ATGACCGAGTACAAGCCCACGGTGCGCCTCGCCACCCGCGACGACGTCCCCAGG
GCCGTACGCACCCTCGCCGCCGCGTTCGCCGACTACCCCGCCACGCGCCACACCG
TCGATCCGGACCGCCACATCGAGCGGGTCACCGAGCTGCAAGAACTCTTCCTCAC
GCGCGTCGGGCTCGACATCGGCAAGGTGTGGGTCGCGGACGACGGCGCCGCGGT
GGCGGTCTGGACCACGCCGGAGAGCGTCGAAGCGGGGGCGGTGTTCGCCGAGAT
CGGCCCGCGCATGGCCGAGTTGAGCGGTTCCCGGCTGGCCGCGCAGCAACAGAT
GGAAGGCCTCCTGGCGCCGCACCGGCCCAAGGAGCCCGCGTGGTTCCTGGCCAC
CGTCGGCGTCTCGCCCGACCACCAGGGCAAGGGTCTGGGCAGCGCCGTCGTGCT
CCCCGGAGTGGAGGCGGCCGAGCGCGCCGGGGTGCCCGCCTTCCTGGAGACCTC
CGCGCCCCGCAACCTCCCCTTCTACGAGCGGCTCGGCTTCACCGTCACCGCCGAC
GTCGAGGTGCCCGAAGGACCGCGCACCTGGTGCATGACCCGCAAGCCCGGTGCC
TGACTCGAGTCTAGAGGGCCCGTTTAAACCCGCTGATCAGCCTCGACTGTGCCTT
CTAGTTGCCAGCCATCTGTTGTTTGCCCCTCCCCCGTGCCTTCCTTGACCCTGGAA
GGTGCCACTCCCACTGTCCTTTCCTAATAAAATGAGGAAATTGCATCGCATTGTC
TGAGTAGGTGTCATTCTATTCTGGGGGGTGGGGGGGGCAGGACAGCAAGGGGG
AGGATTGGGAAGACAATAGCAGGCATGCTGGGGATGCGGTGGGCTCTATGGCTC
GAGTTAATTAACGAGAGCATAATATTGATATGTGCCAAAGTTGTTTCTGACTGAC
TAATAAGTATAATTTGTTTCTATTATGTATAGGTTAAGCTAATTACTTATTTTATA
ATACAACATGACTGTTTTTAAAGTACAAAATAAGTTTATTTTTGTAAAAGAGAGA
ATGTTTAAAAGTTTTGTTACTTTATAGAAGAAATTTTGAGTTTTTGTTTTTTTTTAA
TAAATAAATAAACATAAATAAATTGTTTGTTGAATTTATTATTAGTATGTAAGTG
TAAATATAATAAAACTTAATATCTATTCAAATTAATAAATAAACCTCGATATACA
GACCGATAAAACACATGCGTCAATTTTACGCATGATTATCTTTAACGTACGTCAC
AATATGATTATCTTTCTAGGGTTAAATAATAGTTTCTAATTTTTTTATTATTCAGC
CTGCTGTCGTGAATACCGAGCTCCAATTCGCCCTATAGTGAGTCGTATTACAATT
CACTGGCCGTCGTTTTACAACGTCGTGACTGGGAAAACCCTGGCGTTACCCAACT
TAATCGCCTTGCAGCACATCCCCCTTTCGCCAGCTGGCGTAATAGCGAAGAGGCC
CGCACCGATCGCCCTTCCCAACAGTTGCGCAGCCTGAATGGCGAATGGGACGCG
CCCTGTAGCGGCGCATTAAGCGCGGCGGGTGTGGTGGTTACGCGCAGCGTGACC
GCTACACTTGCCAGCGCCCTAGCGCCCGCTCCTTTCGCTTTCTTCCCTTCCTTTCTC
GCCACGTTCGCCGGCTTTCCCCGTCAAGCTCTAAATCGGGGGCTCCCTTTAGGGT
TCCGATTTAGTGCTTTACGGCACCTCGACCCCAAAAAACTTGATTAGGGTGATGG
TTCACGTAGTGGGCCATCGCCCTGATAGACGGTTTTTCGCCCTTTGACGTTGGAG
TCCACGTTCTTTAATAGTGGACTCTTGTTCCAAACTGGAACAACACTCAACCCTA
TCTCGGTCTATTCTTTTGATTTATAAGGGATTTTGCCGATTTCGGCCTATTGGTTA
AAAAATGAGCTGATTTAACAAAAATTTAACGCGAATTTTAACAAAATATTAACG
CTTACAATTTAGGTGGCACTTTTCGGGGAAATGTGCGCGGAACCCCTATTTGTTT
ATTTTTCTAAATACATTCAAATATGTATCCGCTCATGAGACAATAACCCTGATAA
ATGCTTCAATAATATTGAAAAAGGAAGAGTATGAGTATTCAACATTTCCGTGTCG
CCCTTATTCCCTTTTTTGCGGCATTTTGCCTTCCTGTTTTTGCTCACCCAGAAACGC
TGGTGAAAGTAAAAGATGCTGAAGATCAGTTGGGTGCACGAGTGGGTTACATCG
AACTGGATCTCAACAGCGGTAAGATCCTTGAGAGTTTTCGCCCCGAAGAACGTTT
TCCAATGATGAGCACTTTTAAAGTTCTGCTATGTGGCGCGGTATTATCCCGTATTG
ACGCCGGGCAAGAGCAACTCGGTCGCCGCATACACTATTCTCAGAATGACTTGGT
TGAGTACTCACCAGTCACAGAAAAGCATCTTACGGATGGCATGACAGTAAGAGA
ATTATGCAGTGCTGCCATAACCATGAGTGATAACACTGCGGCCAACTTACTTCTG
ACAACGATCGGAGGACCGAAGGAGCTAACCGCTTTTTTGCACAACATGGGGGAT
CATGTAACTCGCCTTGATCGTTGGGAACCGGAGCTGAATGAAGCCATACCAAAC
GACGAGCGTGACACCACGATGCCTGTAGCAATGGCAACAACGTTGCGCAAACTA
TTAACTGGCGAACTACTTACTCTAGCTTCCCGGCAACAATTAATAGACTGGATGG
AGGCGGATAAAGTTGCAGGACCACTTCTGCGCTCGGCCCTTCCGGCTGGCTGGTT
TATTGCTGATAAATCTGGAGCCGGTGAGCGTGGGTCTCGCGGTATCATTGCAGCA
CTGGGGCCAGATGGTAAGCCCTCCCGTATCGTAGTTATCTACACGACGGGGAGTC
AGGCAACTATGGATGAACGAAATAGACAGATCGCTGAGATAGGTGCCTCACTGA
TTAAGCATTGGTAACTGTCAGACCAAGTTTACTCATATATACTTTAGATTGATTTA
AAACTTCATTTTTAATTTAAAAGGATCTAGGTGAAGATCCTTTTTGATAATCTCAT
GACCAAAATCCCTTAACGTGAGTTTTCGTTCCACTGAGCGTCAGACCCCGTAGAA
AAGATCAAAGGATCTTCTTGAGATCCTTTTTTTCTGCGCGTAATCTGCTGCTTGCA
AACAAAAAAACCACCGCTACCAGCGGTGGTTTGTTTGCCGGATCAAGAGCTACC
AACTCTTTTTCCGAAGGTAACTGGCTTCAGCAGAGCGCAGATACCAAATACTGTT
CTTCTAGTGTAGCCGTAGTTAGGCCACCACTTCAAGAACTCTGTAGCACCGCCTA
CATACCTCGCTCTGCTAATCCTGTTACCAGTGGCTGCTGCCAGTGGCGATAAGTC
GTGTCTTACCGGGTTGGACTCAAGACGATAGTTACCGGATAAGGCGCAGCGGTC
GGGCTGAACGGGGGGTTCGTGCACACAGCCCAGCTTGGAGCGAACGACCTACAC
CGAACTGAGATACCTACAGCGTGAGCTATGAGAAAGCGCCACGCTTCCCGAAGG
GAGAAAGGCGGACAGGTATCCGGTAAGCGGCAGGGTCGGAACAGGAGAGCGCA
CGAGGGAGCTTCCAGGGGGAAACGCCTGGTATCTTTATAGTCCTGTCGGGTTTCG
CCACCTCTGACTTGAGCGTCGATTTTTGTGATGCTCGTCAGGGGGGCGGAGCCTA
TGGAAAAACGCCAGCAACGCGGCCTTTTTACGGTTCCTGGCCTTTTGCTGGCCTT
TTGCTCACATGTTCTTTCCTGCGTTATCCCCTGATTCTGTGGATAACCGTATTACC
GCCTTTGAGTGAGCTGATACCGCTCGCCGCAGCCGAACGACCGAGCGCAGCGAG
TCAGTGAGCGAGGAAGCGGAAGAGCGCCCAATACGCAAACCGCCTCTCCCCGCG
CGTTGGCCGATTCATTAATGCAGCTGGCACGACAGGTTTCCCGACTGGAAAGCGG
GCAGTGAGCGCAACGCAATTAATGTGAGTTAGCTCACTCATTAGGCACCCCAGG
CTTTACACTTTATGCTTCCGGCTCGTATGTTGTGTGGAATTGTGAGCGGATAACA
ATTTCACACAGGAAACAGCTATGACCATGATTACGCCAAGCTCGAAATTAACCCT
CACTAAAGGGAACAAAAGCTGGTACCTCGCGCGACTTGGTTTGCCATTCTTTAGC
GCGCGTCGCGTCACACAGCTTGGCCACAATGTGGTTTTTGTCAAACGAAGATTCT
ATGACGTGTTTAAAGTTTAGGTCGAGTAAAGCGCAAATCTTTTTTAACCCTAGAA
AGATAGTCTGCGTAAAATTGACGCATGCATTCTTGAAATATTGCTCTCTCTTTCTA
AATAGCGCGAATCCGTCGCTGTGCATTTAGGACATCTCAGTCGCCGCTTGGAGCT
CCCGTGAGGCGTGCTTGTCAATGCGGTAAGTGTCACTGATTTTGAACTATAACGA
CCGCGTGAGTCAAAATGACGCATGATTATCTTTTACGTGACTTTTAAGATTTAAC
TCATACGATAATTATATTGTTATTTCATGTTCTACTTACGTGATAACTTATTATAT
ATATATTTTCTTGTTATAGATATCATCAACTTTGTATAGAAAAGTTGGGCTCCGGT
GCCCGTCAGTGGGCAGAGCGCACATCGCCCACAGTCCCCGAGAAGTTGGGGGGA
GGGGTCGGCAATTGAACCGGTGCCTAGAGAAGGTGGCGCGGGGTAAACTGGGAA
AGTGATGTCGTGTACTGGCTCCGCCTTTTTCCCGAGGGTGGGGGAGAACCGTATA
TAAGTGCAGTAGTCGCCGTGAACGTTCTTTTTCGCAACGGGTTTGCCGCCAGAAC
ACAGGTAAGTGCCGTGTGTGGTTCCCGCGGGCCTGGCCTCTTTACGGGTTATGGC
CCTTGCGTGCCTTGAATTACTTCCACCTGGCTGCAGTACGTGATTCTTGATCCCGA
GCTTCGGGTTGGAAGTGGGTGGGAGAGTTCGAGGCCTTGCGCTTAAGGAGCCCC
TTCGCCTCGTGCTTGAGTTGAGGCCTGGCCTGGGCGCTGGGGCCGCCGCGTGCGA
ATCTGGTGGCACCTTCGCGCCTGTCTCGCTGCTTTCGATAAGTCTCTAGCCATTTA
AAATTTTTGATGACCTGCTGCGACGCTTTTTTTCTGGCAAGATAGTCTTGTAAATG
CGGGCCAA
SEQ ID NO: 6. Insertion Sequence: (QPKN01007947.1: 46099-47772)
TAAGAAAactcctgctctccctcctctccctctccctcctctccctctccctctccctct
ccctctccccacggtctccctctccccacggtctccctctccctctctttccacggtctc
cccctgatgctgagccgaagctggactgtactgctgccatctcggctcactgcaacctcc
ctgcctgattctcctgcctcagcctgccgagtgcctgcgattgcaggcgcgtgccgccac
gcctgactggttttcgtatttttttggtggagacggggtttcgctgtgttggccgggctg
gtctccagctcctaatcacgagtgatccgccagccttggcctcccgaggtgccgggattg
cagacggagtctcgttcactcagtgctcaatggtgcccaggctggagtgcagtggcgtga
tctcggcttgctacaacctccacctcccagccgcctgccttggcctcccaaagtgccgag
attgcagcctctgcccggccgccaccccgtctgggaagtgaggagcgtctctgcctggcc
gcccatcgtctgggacgtgaggagcccctctgcctggctgcccagtctggaaagtgagga
gcgtctctgcccggccgccatcccatctaggaagtgaggagcgcctctgccaggccgccc
atcgtctgagatgtggggagcgcctctgccctgccaccccgtctgggatgtgaggagcgt
ctctgcccggccgccccgtctgagaagtgaggagaccctctgcctggcaaccgccccgtc
tgagaagtgaggagcccctccgcccggcagccacaccctctgagaagtgaggagcgtctc
tgcctggcagccaccccgtctgggagggaggtgggggtcagccccctgccccgCCAGCtg
cccatccgggagggaggtggggggtcagccccccgcccggccagccgcctcgtccaggag
gtgaggggcgcctctgcccggccgcccctactgggaagtgaggagcccctctgcctggcc
agccgccccgtcggggagggaggtggggggacagccccccgcccggccagccgccccgtc
cgggaggtgaggggcgcctctgcccggccgcccctactgggaagtgaggagcccctctgc
ccggccaccaccccgtctgggaggtgtactcaacagctcattgagaacgggccatgatga
caatggcggttttgtggaatagaaaggggggaaaggtggggaaaagattgagaaatcgga
tggttgccgtgtctgtgtagaaagaggtagacatgggagacttttcattttgttctgtac
taagaaaaattcttctgccttgggatcctgttgatctgtgaccttacccccaaccctgtg
ctctctgaaacatgtgctgtgtccactcagggttgaatggattaagggtggtgcaagatg
tgctttgttaaacagatgcttgaaggcagcatgctcgttaagagtcatcaccactcccta
atctcaagtacccagggacacaaacactgcggaaggccgcagggtcctctgcctaggaaa
accagagacctttgttcacttatctgctgaccttccctccactattgtcctgtgaccctg
ccaaatccccctctgcgagaaacacccaagaatgatcaataaaaaaaaaaaaaa

### TABLES

TABLE 1
Functional categories of genes showing NHIP associated expression in human cortex, differential
expression in NHIP overexpressing cells, and known ASD risk (from FIG. 4d overlap).

| | GO Terms | | | | | | |
|---|---|---|---|---|---|---|---|
| | | Regulation of | | | | | |
| | | transcription | regulation of | | | Response to | |
| | Chromatin | by RNA | cell | | Rhythmic | decreased | |
| Genes | organization | polymerase II | differentiation | Neurogenesis | process | oxygen levels | Count |
| EP300 | X | X | X | X | X | X | 6 |
| CRE88P | X | X | X | | X | X | 5 |
| SMAD4 | X | X | X | X | | X | 5 |
| RORA | | X | X | X | X | X | 5 |
| HNRNPU | X | X | X | | X | | 4 |
| KMT2A | X | X | X | | X | | 4 |
| NIPBL | X | X | X | X | | | 4 |
| ADNP | | X | X | X | X | | 4 |
| NR1D1 | | X | X | X | X | | 4 |
| ARID18 | X | X | | X | | | 3 |
| PRKCA | X | | X | X | | | 3 |
| RERE | X | X | | X | | | 3 |
| CUX1 | | X | X | X | | | 3 |
| GRIN1 | | X | X | X | | | 3 |
| NR2F1 | | X | X | X | | | 3 |
| SPEN | | X | X | X | | | 3 |
| ASH1L | X | X | | | | | 2 |
| BRD4 | X | X | | | | | 2 |
| CHD2 | X | X | | | | | 2 |
| HCFC1 | X | X | | | | | 2 |
| HUWE1 | X | | | | X | | 2 |
| KAT6A | X | X | | | | | 2 |
| KMT2E | X | | X | | | | 2 |
| CDK13 | | X | X | | | | 2 |
| PCM1 | | | X | X | | | 2 |
| JMJD1C | X | | | | | | 1 |
| KANSL1 | X | | | | | | 1 |
| SETD5 | X | | | | | | 1 |
| CIC | | X | | | | | 1 |
| FOXG1 | | X | | | | | 1 |
| MED13 | | X | | | | | 1 |
| POGZ | | X | | | | | 1 |
| ZNF292 | | X | | | | | 1 |
| TNRC68 | | | X | | | | 1 |
| CPEB4 | | | | | | X | 1 |

TABLE 2
Gene ontology analysis on the overlapped genes among
DGE in brain, DGE in cell, and SFARI ASD genes.

| | | Brain_log2Fold | | | | Cell_P. | Cell_adj. |
|---|---|---|---|---|---|---|---|
| SYMBOL | ID | Change | Brain_pvalue | Brain_padj | Cell_logFC | Value | P. Val |
| NIPBL | 25836 | −0.00684 | 0.001831 | 0.046617 | −0.3762 | 1.89E−05 | 0.002772 |
| MED13 | 9969 | −0.00761 | 0.001372 | 0.041881 | −0.41061 | 0.000124 | 0.005074 |
| WDFY3 | 23001 | −0.0077 | 0.001286 | 0.040682 | −0.68026 | 9.32E−06 | 0.002402 |
| ADNP | 23394 | −0.00826 | 0.000995 | 0.035535 | −0.43457 | 5.50E−05 | 0.003652 |
| ARID1B | 57492 | −0.00848 | 0.000908 | 0.03365 | −0.19268 | 0.002001 | 0.020634 |
| CDK13 | 8621 | −0.00888 | 0.001267 | 0.040245 | −0.34286 | 1.30E−05 | 0.002667 |
| SMAD4 | 4089 | −0.00916 | 0.000458 | 0.02382 | −0.17085 | 0.001019 | 0.013953 |
| ZC3H11A | 9877 | −0.00947 | 0.000491 | 0.024545 | −0.32086 | 7.65E−05 | 0.004051 |
| CHD2 | 1106 | −0.00962 | 0.002126 | 0.049492 | −0.42318 | 1.78E−05 | 0.002772 |
| KAT6A | 7994 | −0.00972 | 0.00086 | 0.032625 | −0.37327 | 3.07E−06 | 0.002157 |
| PCM1 | 5108 | −0.00978 | 0.00044 | 0.023544 | −0.1126 | 0.00518 | 0.036616 |
| SON | 6651 | −0.01016 | 1.55E−05 | 0.004707 | −0.60989 | 4.60E−07 | 0.001507 |
| HCFC1 | 3054 | −0.01031 | 0.001111 | 0.037286 | −0.22933 | 0.0067 | 0.043237 |
| UBN2 | 254048 | −0.01041 | 0.000332 | 0.020376 | −0.75155 | 1.28E−05 | 0.002667 |
| ASH1L | 55870 | −0.01044 | 5.25E−05 | 0.008201 | −0.64357 | 2.99E−06 | 0.002157 |
| JMJD1C | 221037 | −0.01051 | 9.47E−05 | 0.01047 | −0.32015 | 3.29E−05 | 0.003042 |
| HUWE1 | 10075 | −0.01062 | 0.000575 | 0.026554 | −0.43691 | 0.000212 | 0.006257 |
| SETD5 | 55209 | −0.01171 | 0.000233 | 0.017198 | −0.38839 | 1.04E−05 | 0.002402 |
| PRKCA | 5578 | −0.01173 | 0.000124 | 0.012043 | −0.37423 | 0.001448 | 0.017213 |
| HNRNPU | 3192 | −0.01183 | 0.00018 | 0.015032 | −0.15029 | 0.00448 | 0.033571 |
| RERE | 473 | −0.01184 | 8.62E−06 | 0.003535 | −0.33587 | 0.000314 | 0.007646 |
| RORA | 6095 | −0.0119 | 0.001165 | 0.03845 | −0.1853 | 0.002586 | 0.024135 |
| CIC | 23152 | −0.01201 | 0.001538 | 0.043987 | −0.33509 | 0.000335 | 0.007901 |
| KMT2E | 55904 | −0.01219 | 0.000531 | 0.025792 | −0.20107 | 0.001002 | 0.0138 |
| ZNF292 | 23036 | −0.01227 | 6.65E−05 | 0.009216 | −0.17219 | 0.002117 | 0.021296 |
| SYNE1 | 23345 | −0.01241 | 6.18E−05 | 0.008943 | −0.35909 | 0.000672 | 0.011064 |
| TNRC6B | 23112 | −0.01248 | 1.85E−05 | 0.004873 | −0.50571 | 2.78E−05 | 0.002958 |
| PRR12 | 57479 | −0.01257 | 0.000409 | 0.022513 | −0.22967 | 0.001297 | 0.016123 |
| EP300 | 2033 | −0.01271 | 1.84E−05 | 0.004873 | −0.52373 | 1.84E−05 | 0.002772 |
| POGZ | 23126 | −0.01295 | 0.000162 | 0.014246 | −0.31024 | 9.13E−05 | 0.004326 |
| KANSL1 | 284058 | −0.01308 | 0.000542 | 0.026096 | −0.19986 | 0.000658 | 0.011004 |
| CREBBP | 1387 | −0.01315 | 3.55E−05 | 0.007048 | −0.54917 | 1.48E−06 | 0.001651 |
| CUX1 | 1523 | −0.01353 | 0.000129 | 0.012169 | −0.39031 | 2.07E−05 | 0.002805 |
| SPEN | 23013 | −0.01391 | 0.000754 | 0.030276 | −0.55901 | 0.000571 | 0.010358 |
| BRD4 | 23476 | −0.01483 | 4.43E−06 | 0.002563 | −0.46617 | 2.03E−06 | 0.001759 |
| KMT2A | 4297 | −0.01525 | 2.62E−05 | 0.006 | −0.90277 | 1.27E−06 | 0.001651 |
| GPX1 | 2876 | −0.01613 | 0.001218 | 0.039223 | 0.203382 | 0.002224 | 0.021902 |
| CPEB4 | 80315 | −0.0166 | 6.08E−06 | 0.003067 | −0.71794 | 9.62E−06 | 0.002402 |
| KCNQ2 | 3785 | −0.017 | 0.00028 | 0.019024 | 0.377036 | 0.000237 | 0.006613 |
| ARHGAP32 | 9743 | −0.01709 | 0.000264 | 0.018279 | −0.40742 | 0.000141 | 0.005355 |
| FOXG1 | 2290 | −0.01803 | 5.65E−05 | 0.008504 | −0.13047 | 0.006512 | 0.042443 |
| NR2F1 | 7025 | −0.01839 | 1.86E−06 | 0.001737 | 0.217604 | 0.006027 | 0.040409 |
| LRRC4 | 64101 | −0.02012 | 6.38E−05 | 0.0091 | −0.57006 | 0.000637 | 0.010871 |
| GRIN1 | 2902 | −0.02243 | 3.31E−06 | 0.002284 | −0.81832 | 0.006422 | 0.042007 |
| NR1D1 | 9572 | −0.02545 | 0.000208 | 0.016396 | −0.57072 | 0.001503 | 0.017611 |

What is claimed is:

1. A method for determining a risk of an offspring for developing an autism spectrum disorder (ASD), the method comprising:
detecting in a biological sample obtained from the offspring, mother or potential mother of the offspring expression and/or DNA methylation of a neuronal hypoxia inducible, placental associated (NHIP) gene, wherein decreased expression and/or decreased methylation of the NHIP gene compared to a control sample indicates an increased risk of the offspring for developing an ASD.

2. The method of claim 1, wherein the method further comprises obtaining a biological sample from the mother or potential mother.

3. The method of claim 1, wherein the biological sample is selected from the group consisting of blood, serum, plasma, or saliva from the mother, and placenta, cord blood, blood, saliva and brain from the offspring.

4. The method of claim 1, wherein the mother or potential mother has a child with an ASD.

5. The method of claim 1, wherein the mother or potential mother has a familial history of ASD.

6. The method of claim 1, wherein the offspring is a fetus or child.

7. The method of claim 1, wherein the control sample is selected from a mother or potential mother having an offspring without an ASD or an offspring exhibiting typical development.

8. The method of claim 1, wherein the detecting step comprises detecting DNA methylation of the NHIP genetic locus, the chr22q13.33 hypomethylated block, or both.

9. The method of claim 8, wherein lower DNA methylation levels indicates an increased risk of the offspring for developing an ASD.

10. The method of claim 1, wherein detecting expression of the NHIP gene comprises detecting an RNA expressed by the NHIP gene or detecting a peptide encoded by the RNA.

11. The method of claim 10, wherein the RNA is transcribed from an open reading frame comprising the DNA sequence
(SEQ ID NO: 2)
ATGGTGAGAGGAGAGGCCACCGCACGAACGGAAGAAGCGATGGAGACGG
TCTTTACGACC.

12. The method of claim 10, wherein detecting an RNA expressed by the NHIP gene is selected from amplifying the RNA, quantifying the RNA, or sequencing the RNA.

13. The method of claim 10, wherein detecting a peptide encoded by the RNA is selected from i) contacting the peptide with a primary antibody that binds the peptide and detecting the primary antibody with a labeled secondary antibody, ii) linking the peptide to a detectable label, or iii) by immunostaining.

14. The method of claim 10, wherein the peptide comprises the amino acid sequence MVRGEATARTEEAMETVFTT (SEQ ID NO:1).

15. The method of claim 1, further comprising administering a vitamin to the mother or potential mother if the mother is homozygous for a structural variant inserted about 15 Kbp upstream from the start site of the chr22q13.33 hypomethylated block.

16. The method of claim 15, wherein the vitamin is administered during the first month of pregnancy.

17. The method of claim 15, wherein the vitamin comprises a, one or more, or a plurality of dietary methyl group(s).

18. The method of claim 1, wherein the NHIP gene is hypomethylated.

19. The method of claim 1, wherein the biological sample is homozygous for a structural variant insertion (chr22: 49029657, hg38) upstream of the 22q13.33 locus.

20. A method for detecting an NHIP peptide in a subject, the method comprising:
obtaining a biological sample from the subject; and
detecting the presence of the NHIP peptide by contacting the biological sample with an anti-NHIP antibody and detecting binding between the NHIP peptide and the antibody.

21. The method of claim 20, wherein the subject is a mother or potential mother of an offspring at risk for developing an ASD.

22. A method for preventing an autism spectrum disorder (ASD) in an offspring, the method comprising:
administering a vitamin to the mother of the offspring before and/or during pregnancy, wherein the mother has decreased expression and/or DNA methylation of the NHIP gene in a biological sample compared to a control sample.

23. A method for preventing or reducing a risk of an offspring for developing an autism spectrum disorder (ASD), the method comprising:
i) selecting a mother or potential mother of the offspring, wherein the mother or potential mother is selected based on having decreased expression and/or DNA methylation of the NHIP gene in a biological sample compared to a control sample; and
ii) administering a vitamin to the mother or potential mother before and/or during pregnancy, thereby preventing or reducing the risk that the offspring develops an ASD.

24. The method of claim 20, wherein the biological sample is selected from the group consisting of blood, serum, plasma, or saliva from the mother, and placenta, cord blood, blood, saliva and brain from the offspring.

25. The method of claim 22, wherein the control sample is selected from a mother or potential mother having an offspring without an ASD or an offspring exhibiting typical development.

26. A method for preventing or reducing a risk of an offspring for developing an autism spectrum disorder (ASD), the method comprising:
administering a therapeutically effective amount of an NHIP gene, an NHIP RNA, or an NHIP peptide, to the mother of the offspring before and/or during pregnancy, thereby preventing or reducing the risk of the offspring for developing an ASD.

27. A plasmid or vector comprising the NHIP gene, or DNA encoding an NHIP RNA or peptide.

28. The plasmid or vector of claim 27, further comprising nucleic acid sequences that regulate transcription and/or translation of the NHIP RNA.

29. An in vitro method for increasing cell proliferation, comprising transfecting a cell with the plasmid or vector of claim 27.

30. A method for regulating gene expression, comprising transfecting a cell with the plasmid or vector of claim 27, and detecting differential expression of one or more genes.

31. An isolated peptide comprising an amino acid sequence having at least about 80% sequence identity to SEQ ID NO:1.

32. A fusion protein comprising the peptide of claim 31.

33. A kit comprising reagents for detecting expression of an NHIP RNA or NHIP peptide.

34. An array comprising one or more nucleic acid sequences or probes that are capable of hybridizing to an NHIP RNA.

35. An array comprising one or more agents that bind to an NHIP peptide immobilized on a solid support.

36. The array of claim 35, wherein the one or more agents comprise an antigen binding protein that specifically binds to the NHIP peptide.

37. A method for sequencing an NHIP gene sequence, comprising amplifying all or part of an NHIP gene from a biological sample obtained from a subject using a set of primers to produce amplified nucleic acid; and sequencing the amplified nucleic acid.

38. The method of claim 37, wherein the subject is a mother or potential mother of an offspring at risk for developing an ASD.
